(19) United States
(12) Patent Application Publication
(10) Pub. No.: US 2009/0328255 A1
(43) Pub. Date: Dec. 31, 2009

(54) NITROGEN LIMITATION ADAPTABILITY GENE AND PROTEIN AND MODULATION THEREOF

(75) Inventors: Steven Rothstein, Guelph (CA); Mingsheng Peng, Guelph (CA); Yong-Mei Bi, Guelph (CA)

Correspondence Address: PILLSBURY WINTHROP SHAW PITTMAN, LLP, P.O. BOX 10500, MCLEAN, VA, 22102, US

(73) Assignee: University of Guelph

(21) Appl. No.: 12/304,987

(22) PCT Filed: Jun. 13, 2007
(86) PCT No.: PCT/CA2007/001019

§ 371 Date: Jun. 15, 2009

Related U.S. Application Data
(60) Provisional application No. 60/812,981, filed on Jun. 13, 2006.

(51) Int. Cl. C12N 15/82; C07H 21/04; C12N 15/63; C12N 5/04; A01H 1/00; A01H 5/10; A01H 5/00; C07K 16/00

(52) U.S. Cl. 800/279; 435/468; 536/24.1; 435/320.1; 435/419; 800/278; 800/293; 800/298; 530/387.9

(57) ABSTRACT

The present invention relates to a nitrogen-regulated RING-like ubiquitin E3 ligase gene required for sugar sensing and the modulation of the expression of this gene to modulate a characteristic in a plant. The RING-like ubiquitin E3 ligase of the present invention is involved in mediating nitrogen limitation adaptive responses in plants and its expression is influenced by nitrogen status. Increased expression of this or substantially similar genes can produce plants with improved nitrogen utilization and increased yield.

### FIELD OF THE INVENTION

The present invention relates to methods of modulating agronomic traits in plants by modulating the expression of a RING-Type ubiquitination ligase in the plant cells. In particular the present invention relates to methods of improving nitrogen utilization in plants. The present invention also pertains to nucleic acid molecules isolated from Arabidopsis thaliana comprising nucleotide sequences that encode proteins that mediate nitrogen limitation adaptibility and, ultimately, can modulate responses to nitrogen limitation including nitrogen recycling, anthocyanin production, sugar mobilization, and reduced photosynthesis.

### BACKGROUND OF THE INVENTION

Improvement of the agronomic characteristics of crop plants has been ongoing since the beginning of agriculture. Most of the land suitable for crop production is currently being used. As human populations continue to increase, improved crop varieties will be required to adequately provide the world's food and feed (Trewavas (2001) Plant Physiol. 125: 174-179). To avoid catastrophic famines and malnutrition, future crop cultivars will need to have improved yields with equivalent farm inputs. These cultivars will need to more effectively withstand adverse conditions such as drought, soil salinity or disease, which will be especially important as marginal lands are brought into cultivation. Finally, it will be desirable to have cultivars with altered nutrient composition to enhance human and animal nutrition, and to enable more efficient food and feed processing. For all these traits, identification of the genes controlling phenotypic expression of traits of interest will be crucial in accelerating development of superior crop germplasm by conventional or transgenic means.

A number of highly-efficient approaches are available to assist identification of genes playing key roles in expression of agronomically-important traits. These include genetics, genomics, bioinformatics, and functional genomics. Genetics is the scientific study of the mechanisms of inheritance. By identifying mutations that alter the pathway or response of interest, classical (or forward) genetics can help to identify the genes involved in these pathways or responses. For example, a mutant with enhanced susceptibility to disease may identify an important component of the plant signal transduction pathway leading from pathogen recognition to disease resistance. Genetics is also the central component in improvement of germplasm by breeding. Through molecular and phenotypic analysis of genetic crosses, loci controlling traits of interest can be mapped and followed in subsequent generations. Knowledge of the genes underlying phenotypic variation between crop accessions can enable development of markers that greatly increase efficiency of the germplasm improvement process, as well as open avenues for discovery of additional superior alleles.

Genomics is the system-level study of an organism's genome, including genes and corresponding gene —RNA and proteins. At a first level, genomic approaches have provided large datasets of sequence information from diverse plant species, including full-length and partial cDNA sequences, and the complete genomic sequence of a model plant species, Arabidopsis thaliana. Recently, the first draft sequence of a crop plant's genome, that of rice (Oryza sativa), has also become available. Availability of a whole genome sequence makes possible the development of tools for system-level study of other molecular complements, such as arrays and chips for use in determining the complement of expressed genes in an organism under specific conditions. Such data can be used as a first indication of the potential for certain genes to play key roles in expression of different plant phenotypes.

Bioinformatics approaches interface directly with first-level genomic datasets in allowing for processing to uncover sequences of interest by annotative or other means. Using, for example, similarity searches, alignments and phylogenetic analyses, bioinformatics can often identify homologs of a gene product of interest. Very similar homologs (eg. >˜90% amino acid identity over the entire length of the protein) are very likely orthologs, i.e. share the same function in different organisms.

Functional genomics can be defined as the assignment of function to genes and their products. Functional genomics draws from genetics, genomics and bioinformatics to derive a path toward identifying genes important in a particular pathway or response of interest. Expression analysis, for example, uses high density DNA microarrays (often derived from genomic-scale organismal sequencing) to monitor the mRNA expression of thousands of genes in a single experiment. Experimental treatments can include those eliciting a response of interest, such as the disease resistance response in plants infected with a pathogen. To give additional examples of the use of microarrays, mRNA expression levels can be monitored in distinct tissues over a developmental time course, or in mutants affected in a response of interest. Proteomics can also help to assign function, by assaying the expression and post-translational modifications of hundreds of proteins in a single experiment.

Proteomics approaches are in many cases analogous to the approaches taken for monitoring mRNA expression in microarray experiments. Protein-protein interactions can also help to assign proteins to a given pathway or response, by identifying proteins that interact with known components of the pathway or response. For functional genomics, protein-protein interactions are often studied using large-scale yeast two-hybrid assays. Another approach to assigning gene function is to express the corresponding protein in a heterologous host, for example the bacterium Escherichia coli, followed by purification and enzymatic assays.

Demonstration of the ability of a gene-of-interest to control a given trait may be derived, for example, from experimental testing in plant species of interest. The generation and analysis of plants transgenic for a gene of interest can be used for plant functional genomics, with several advantages. The gene can often be both overexpressed and underexpressed (“knocked out”), thereby increasing the chances of observing a phenotype linking the gene to a pathway or response of interest. Two aspects of transgenic functional genomics help lend a high level of confidence to functional assignment by this approach. First, phenotypic observations are carried out in the context of the living plant. Second, the range of phenotypes observed can be checked and correlated with observed expression levels of the introduced transgene. Transgenic functional genomics is especially valuable in improved cultivar development. Only genes that function in a pathway or response of interest, and that in addition are able to confer a desired trait-based phenotype, are promoted as candidate genes for crop improvement efforts. In some cases, transgenic lines developed for functional genomics studies can be directly utilized in initial stages of product development.

Another approach towards plant functional genomics involves first identifying plant lines with mutations in specific genes of interest, followed by phenotypic evaluation of the consequences of such gene knockouts on the trait under study. Such an approach reveals genes essential for expression of specific traits.

Genes identified through functional genomics can be directly employed in efforts towards germplasm improvement by transgenic means, as described above, or used to develop markers for identification of tracking of alleles-of-interest in mapping and breeding populations. Knowledge of such genes may also enable construction of superior alleles non-existent in nature, by any of a number of molecular methods.

Rapid increases in yield over the last 80 years in row crops have been due in roughly equal measure to improved genetics and improved agronomic practices. In particular, in a crop like maize, the combination of high yielding hybrids and the use of large amounts of nitrogen fertilizer have under ideal conditions allowed for yields of greater than 440 bu/acre. However, the use of large amounts of nitrogen fertilizer has negative side-effects primarily around increasing cost of this input to the farmer and cost to the environment since nitrate pollution is a major problem in many agricultural areas contributing significantly to the degradation of both fresh water and marine environments. Developing crop genetics that use nitrogen more efficiently through an understanding of the role of genotype on nitrogen use would be highly advantageous in reducing producer input costs as well as environmental load. This is particularly important for a crop like corn which is grown using a high level of nitrogen fertilizer.

Nitrogen use efficiency can be defined in several ways, although the simplest is yield/N supplied. There are two stages in this process: first, the amount of available nitrogen that is taken up, stored and assimilated into amino acids and other important nitrogenous compounds; second, the proportion of nitrogen that is partitioned to the seed, resulting in final yield. A variety of field studies have been performed on various agriculturally important crops to study this problem (Lawlor D W et al 2001 in Lea P J, Morot-Gaudry J F, eds. Plant Nitrogen. Berlin: Springer-Verlag 343-367; Lafitte H R and Edmeades G O 1994 Field Crops Res 39, 15-25; Lawlor D W 2002 J Exp Bot. 53, 773-87; Moll R H et al 1982 Agron J 74, 562-564). These experiments have demonstrated that there is a genetic component to nitrogen use efficiency, but have not proved satisfactory in determining which genes are important for this process. In addition, corn breeders have generally not targeted the maintenance of yield under limiting nitrogen fertilizer. These types of field experiments on nitrogen use are difficult for a variety of reasons including a lack of uniformity of accessible nitrogen in a test field or between field sites under any treatment regime and the interplay of other environmental factors that make experiments difficult to interpret.

In plants, nitrogen is involved in two roles. First, nitrogen affects plant biomass and crop yield markedly as an essential macronutrient (Lam H M et al (1996) Annu. Rev. Plant Physiol. Plant Mol. Biol. 47, 569-593) Second, as an important signal, nitrogen regulates the expression of many genes involved in nitrogen and carbon metabolism (Crawford N M (1995) Plant Cell 7, 859-868; Stitt M (1999) Curr. Opin. Plant Biol. 2, 178-186), and modulates plant development, such as root branching and development, leaf growth, shoot branching and flowering time (Crawford N M & Forde B G (2002). To achieve optimal growth and development, plants must acquire sufficient nitrogen nutrient from the soil, with the sufficient amount of nitrogen varying between two and five percent of the plant dry weight depending on the plant species and developmental stage (Marschner, 1995). However, because the nitrogen content in the soil is frequently reduced by many abiotic and biotic factors such as soil erosion, rainwater leaching, and microbe consumption (Good et al., 2004), plants are frequently subjected to a nitrogen limitation growth condition. Therefore, nitrogen limitation adaptability is an important survival strategy for plants to successfully finish their life cycles to produce offspring, rather than dying early and barren when nitrogen availability is limited. In crop plants, this nitrogen limitation adaptability has been found to be positively related to their yields. Tollenaar and Wu (1999) reported that increasing maize cultivars' tolerance to nitrogen limitation contributed significantly to the genetic improvement of maize yields over the last several decades. Many studies have demonstrated that newer released maize hybrids could grow more vigorously and produce higher yields than do older ones when grown under nitrogen limitation growth conditions, indicating that the newer maize hybrids have the stronger nitrogen limitation adaptability than do older ones (Castleberry et al., 1984; Duvick, 1984, 1997; McCullough et al., 1994; Ding et al., 2005). This suggests that enhancing crop cultivars' adaptability to nitrogen limitation could increase crop yield and possibly allow for a reduction in the amount of nitrogen fertilizer needed.

Strengthening crop cultivars' nitrogen limitation adaptability is important for current agriculture practice, in which large amounts of nitrogen fertilizer are applied to crops to increase their yield (Frink et al., 1999), and in which more than 50% of applied nitrogen nutrients are lost from the crop-soil system (Peoples et al., 1995). Thus, the use of large amounts of nitrogen fertilizers inevitably increases the cost of crop production and also leads to a significant level of nitrogen pollution (Good et al., 2004). Developing cultivars with an enhanced adaptability to nitrogen limitation might make it possible to reduce this while maintaining crop yield and decrease the environmental footprint of production agriculture (Ding et al., 2005).

The molecular mechanism by which plants adapt to the nitrogen limitation growth condition has not been delineated, and the physiological and biochemical factors specifically involved in plant adaptation to nitrogen limitation have not been studied systematically. However, a number of studies about the effect of nitrogen stress on plant growth and development have been done and from these, some of the expected plant nitrogen limitation adaptive responses can be inferred. These include the reduction of growth and photosynthesis, remobilization of nitrogen from old, mature organs to actively growing ones, and an accumulation of abundant anthocyanin (Khamis et al., 1990; Geiger et al., 1999; Ding et al., 2005; Mei and Thimann, 1984; Ono et al., 1996; Bongue-Bartelsman and Phillops, 1995; Chalker-Scott, 1999; Diaz et al., 2006). In addition, the following findings suggest that plants are equipped with molecular mechanisms governing their adaptability to nitrogen limitation. In Arabidopsis, the transcript of NRT2.1, a high affinity nitrate transporter, was increased significantly by nitrate limitation (Filleur et al., 2001). Todd et al. (2004) found that the expression of a MYB-like gene AtNsr1 was markedly and specifically up-regulated by nitrogen deficiency. Recently, Diaz et al. (2006) reported that growing Arabidopsis plants under low nitrogen conditions resulted in chlorophyll breakdown in old rosette leaves, and anthocyanin accumulation in whole rosette. Fifteen quantitative trait loci (QTLs) were identified in the control of these nitrogen limitation caused growth responses (Diaz et al., 2006). However, no mutant defective in developing the adaptive responses to nitrogen limitation has been found. Consequently, nothing is yet known about the molecular mechanism controlling this phenomenon.

Anthocyanins are a variety of phenylpropanoids, a class of plant-derived organic compounds that are biosynthesized from the amino acid phenylalanine. Phenylpropanoids have a wide variety of functions, including defense against herbivores, microbial attack, or other sources of injury; as structural components of cell walls (i.e. lignin); as protection from ultraviolet light; as pigments (e.g. anthocyanins); and as signaling molecules. Anthocyanin biosynthesis begins with the condensation of p-coumaroyl-CoA and malonyl-CoA by the enzyme chalcone synthase (CHS) to produce the intermediate chalcone. P-coumaroyl-CoA represents an important branch point in phenylproanoid metabolism as it is an intermediate in the production of both anthocyanins and lignin. Use of p-coumaroyl-CoA by CHS drives phenylpropanoid biosynthesis toward flavanoids and anthocyanins, whereas use of p-coumaroyl-CoA by several other enzymes leads to lignin biosynthesis. Anthocyanins are generally not present in the leaf until the breaking down the chlorophyll, during which time the plant begins to synthesize the anthocyanin, presumably for photoprotection during nitrogen translocation.

Protein ubiquitination has been known to play central roles in regulating numerous cellular processes in eukaryotes. First, protein ubiquitination pathway targets various substrates such as nuclear transcription factors, abnormal cytoplasmic proteins, and short-lived regulatory proteins for degradation by the 26S proteasome (Glickman and Ciechanover, 2002). Second, modification of proteins with ubiquitin also regulates protein localization, activity, interacting partners, and functions in a proteasome-independent manner (Schnell and Hicke, 2003; Sun and Chen, 2004).

RING-type ubiquitin E3 ligases are responsible for targeting specific substrate proteins for ubiquitination. The RING domain is a C3HC4 type Zn-finger which binds two atoms of zinc and may be involved in mediating protein-protein interactions. In Arabidopsis, functional characterization of some RING-containing proteins such as COP1 and SINATA5 suggests that the biological function of the RING domain is to participate in ubiquitin-dependent protein degradation (Moon et al, 2004), and thus plays a central and essential role in eukaryotic cellular regulation (Glickman and Ciechanover, 2002). Stone et al. (2005) reported that the Arabidopsis genome encodes 469 putative RING-containing proteins, which can be grouped into eight types (Stone et al., 2005). Nevertheless, it is not clear if all RING finger genes are E3 ubiquitin ligases (Moon et al., 2005).

In plants, the in vivo function of RING-type ubiquitin ligases remains very poorly defined, with the Arabidopsis genome encoding a predicted 469 RING domain proteins (Stone et al. 2005).

### SUMMARY OF THE INVENTION

In an attempt to determine the molecular mechanisms controlling nitrogen limitation adaptability in plants, the present inventors have isolated and characterized an Arabidopsis mutant, called lines (low inorganic nitrogen-induced early senescence), which has lost its ability to adapt to nitrogen limitation. When supplied with insufficient inorganic nitrogen nutrition (nitrate or ammonium), the lines mutant plants failed to develop the essential nitrogen limitation adaptive responses, and consequently senesced much earlier and more rapidly than did wild type plants. This low nitrogen induced, early senescence phenotype could be rescued by supplying the lines plants with a high dosage of nitrogen fertilizer. Detailed physiological, biochemical and molecular analysis demonstrated that when the mutant lines plants were supplied with limited nitrogen nutrient (3 mM nitrate), they were impaired in the development of an entire set of essential nitrogen limitation adaptive responses, and thus failed to acclimatize to the nitrogen limitation growth condition.

The wild type LINES gene (At1g02860) was further identified through a map-based cloning approach and is predicted to encode a RING-type ubiquitin ligase. This suggests that the functional LINES protein participates in protein ubiquitination mediated degradation or modification of a key negative regulator(s) in the Arabidopsis nitrogen limitation signaling pathway. The truncated protein encoded by the lines mutant lacks the RING domain is impaired in the ability to adapt to nitrogen limitation. Thus, the inventors have provided the first insight into the molecular mechanism controlling a plant's adaptability to nitrogen limitation.

The adaptability to nitrogen limitation is an essential trait for plants and is positively correlated with crop yield. Numerous biotic and abiotic factors that consume nitrogen in the soil frequently create a nitrogen limitation growth condition. To cope with this, plants have evolved a suite of nitrogen limitation adaptive responses. However, knowledge is limited on the physiological and biochemical changes involved in these adaptive responses, and nothing has previously been known about the molecular mechanism governing plant adaptability to nitrogen limitation. The RING domain protein disclosed here is involved in mediating the adaptive response of plants to nitrogen limitation.

Accordingly, the present invention relates to a method of modulating a characteristic in a plant or plant cell comprising modulating expression of a RING-type ubiquitin E3 ligase in the plant or plant cell. In an embodiment of the invention, the expression of the RING-type ubiquitin E3 ligase is modulated by administering, to the cell, an effective amount of an agent that can modulate the expression levels of a RING-type ubiquitin E3 ligase gene in the plant cell. In a further embodiment of the invention, the agent enhances the expression levels of a RING-type ubiquitin E3 ligase in the plant cell.

The characteristic to be modulated in the plant may be any agronomic trait of interest. In an embodiment of the invention, the characteristic is any that is affected by nitrogen, carbon and/or sulfur metabolism, biosynthesis of lipids, perception of nutrients, nutritional adaptation, electron transport and/or membrane associated energy conservation. In a further embodiment of the invention, the characteristic is selected from one or more of nitrogen utilization, yield, cell growth, reproduction, photosynthesis, nitrogen assimilation, disease resistance, differentiation, signal transduction, gene regulation, abiotic stress tolerance and nutritional composition. In a still further embodiment of the invention the modulated characteristic is an increase or improvement in one or more of nitrogen utilization, yield, cell growth, reproduction, photosynthesis, nitrogen assimilation, lignin biosynthesis, anthocyanin biosynthesis, disease resistance, differentiation, signal transduction, gene regulation abiotic stress tolerance and nutritional composition.

The plant or plant cell may be from any plant wherein one wishes to modulate a characteristic. In an embodiment of the invention, the plant cell is a dicot, a gymnosperm or a monocot. In one embodiment, the dicot is selected from the group consisting of soybean, tobacco or cotton. In a further embodiment of the invention, the monocot is selected from maize, wheat, barley, oats, rye, millet, sorghum, triticale, secale, einkorn, spelt, emmer, teff, milo, flax, gramma grass, Tripsacum sp. and teosite.

In an embodiment of the invention, the agent that can modulate the expression levels of a RING-type ubiquitin E3 ligase gene in a plant cell comprises:
(a) a nucleotide sequence of SEQ ID NOs:1, 3, 5, 7, 9 or a fragment or domain thereof;
(b) a nucleotide sequence encoding a polypeptide of any one of SEQ ID NOs: 2, 4, 6, 8, 10, a fragment or domain thereof;
(c) a nucleotide sequence having substantial similarity to (a) or (b);
(d) a nucleotide sequence capable of hybridizing to (a), (b) or (c)
(e) a nucleotide sequence complementary to (a), (b), (c) or (d); or
(f) a nucleotide sequence that is the reverse complement of (a), (b), (c) or (d).

In a preferred embodiment of the invention the modulated characteristic is an increase or improvement in one or more of nitrogen utilization, yield, cell growth, reproduction, photosynthesis, nitrogen assimilation, lignin biosynthesis, anthocyanin biosynthesis, disease resistance, differentiation, signal transduction, gene regulation abiotic stress tolerance and nutritional composition.

In a particular embodiment, the present invention relates to a method of improving nitrogen utilization in a plant or plant cell comprising enhancing expression of a RING-type ubiquitin E3 ligase gene in the plant or plant cell. Improving nitrogen utilization in a plant will allow for reduced amounts of nitrogen fertilizer to be applied to the plant with a concomitant reduction in costs to the farmer and cost to the environment since nitrate pollution is a major problem in many agricultural areas contributing significantly to the degradation of both fresh water and marine environments. Furthermore, improving nitrogen utilization may allow for the cultivation of new varieties and species in environments that are otherwise unsuitable for cultivation of said new varieties and species.

In an embodiment of the invention, the agent that enhances the expression levels of a RING-type ubiquitin E3 ligase gene in the plant cell comprises a nucleic acid molecule encoding a RING-type ubiquitin E3 ligase. In an embodiment of the invention, the agent that enhances the expression levels of a RING-type ubiquitin E3 ligase gene in a plant cell comprises:
(a) a nucleotide sequence of SEQ ID NOs:1, 5, 7, 9 or a fragment or domain thereof;
(b) a nucleotide sequence encoding a polypeptide of any one of SEQ ID NOs: 2, 6, 8, 10, a fragment or domain thereof;
(c) a nucleotide sequence having substantial similarity to (a) or (b);
(d) a nucleotide sequence capable of hybridizing to (a), (b) or (c)
(e) a nucleotide sequence complementary to (a), (b), (c) or (d); or
(f) a nucleotide sequence that is the reverse complement of (a), (b), (c) or (d).

In a specific embodiment, the substantial similarity is at least about 65% identity, specifically about 80% identity, specifically 90%, and more specifically at least about 95% sequence identity to the nucleotide sequence listed as SEQ ID NO:1, or a fragment or domain thereof.

In a one embodiment, the sequence having substantial similarity to the nucleotide sequence of SEQ ID NO:1, a fragment or domain thereof, is from a plant. In a specific embodiment, the plant is a dicot. In a more specific embodiment, the dicot is selected from the group consisting of soybean, tobacco, poplar, or cotton. In another specific embodiment, the plant is a gymnosperm. In another specific embodiment, the plant is a monocot. In a more specific embodiment, the monocot is a cereal. In a more specific embodiment, the cereal may be, for example, maize, wheat, barley, oats, rye, millet, sorghum, triticale, secale, einkorn, spelt, emmer, teff, milo, flax, gramma grass, Tripsacum sp., or teosinte.

In a specific embodiment, the isolated nucleic acid comprises or consists of a nucleotide sequence capable of hybridizing to a nucleotide sequence listed in SEQ ID NO:1 or a fragment or domain thereof. In a specific embodiment, hybridization allows the sequence to form a duplex at medium or high stringency. Embodiments of the present invention also encompass a nucleotide sequence complementary to a nucleotide sequence of SEQ ID NO:1 or a fragment or domain thereof. Embodiments of the present invention further encompass a nucleotide sequence complementary to a nucleotide sequence that has substantial similarity or is capable of hybridizing to a nucleotide sequence of SEQ ID NO:1 or a fragment or domain thereof.

In a specific embodiment, the nucleotide sequence having substantial similarity is an allelic variant of the nucleotide sequence of SEQ ID NO:1 a fragment or domain thereof. In an alternate embodiment, the sequence having substantial similarity is a naturally occurring variant. In another alternate embodiment, the sequence having substantial similarity is a polymorphic variant of the nucleotide sequence of SEQ ID NO:1 or a fragment or domain thereof.

In a specific embodiment, the isolated nucleic acid contains a plurality of regions having the nucleotide sequence of SEQ ID NO:1 or exon or domain thereof.

In a specific embodiment, the sequence having substantial similarity contains a deletion or insertion of at least one nucleotide. In a more specific embodiment, the deletion or insertion is of less than about thirty nucleotides. In a most specific embodiment, the deletion or insertion is of less than about five nucleotides.

In a specific embodiment, the sequence of the isolated nucleic acid having substantial similarity comprises or consists of a substitution in at least one codon. In a specific embodiment, the substitution is conservative.

In a further embodiment of the invention, the nucleic acid molecule comprises the sequence of the AT1g02860 gene of SEQ ID NO:1 or a functional fragment thereof. In a still further embodiment of the invention, the nucleic acid molecule comprises a sequence that hybridizes under medium stringency conditions to the AT1g02860 gene of SEQ ID NO:1 or a functional fragment thereof. In another embodiment of the present invention, the nucleic acid molecule is derived from the nucleotide sequence of the AT1g02860 gene of SEQ ID NO:1 and has a nucleotide sequence comprising codons specific for expression in plants. In yet another embodiment of the invention, the nucleic acid is the lines mutation of the AT1g02860 gene comprising the sequence of SEQ ID NO:3, or a functional fragment thereof, encoding the polypeptide of SEQ ID NO:4. In yet another embodiment of the invention, the nucleic acid molecule is the Arabidopsis homologue of the AT1g02860 gene comprising the sequence of the AT2g38920 gene of SEQ ID NO:5, or a functional fragment thereof, encoding the polypeptide of SEQ ID NO:6. In yet another embodiment of the invention, the nucleic acid molecule is the rice homologue of the AT1g02860 gene comprising the nucleotide sequence of SEQ ID NO:7, or a functional fragment thereof, encoding the polypeptide of SEQ ID NO:8. In yet another embodiment of the invention, the nucleic acid molecule is a rice homologue of the AT1g02860 gene comprising the nucleotide sequence of SEQ ID NO:9, or a functional fragment thereof, encoding the polypeptide of SEQ ID NO:10.

In another embodiment, the modulated characteristic is a decrease or reduction in one or more of nitrogen utilization, yield, cell growth, reproduction, photosynthesis, nitrogen assimilation, lignin biosynthesis, anthocyanin biosynthesis, disease resistance, differentiation, signal transduction, gene regulation abiotic stress tolerance and nutritional composition. In such an embodiment, the agent will inhibit the expression of a RING-like ubiquitin E3 ligase. Such agents are described in Section VI and can be selected from antisense oligonucleotides, aptamers and double stranded RNA molecules or RNA induced silencing complexes. Such agents can interfere with the expression of the RING-like ubiquitin ligases of SEQ ID NOs:1, 5, 7 or 9.

In an embodiment of the present invention, when the agent is a nucleic acid sequence, the nucleic acid sequence is expressed in a specific location or tissue of the plant. The location or tissue is for example, but not limited to, epidermis, root, vascular tissue, meristem, cambium, cortex, pith, leaf and/or flower. In an alternative embodiment, the location or tissue is a seed.

Embodiments of the present invention also relate to use of a shuffled nucleic acid molecule for modulating a characteristic in a plant cell, said shuffled nucleic acid molecule containing a plurality of nucleotide sequence fragments, wherein at least one of the fragments encodes a RING-like ubiquitin E3 ligase and wherein at least two of the plurality of sequence fragments are in an order, from 5′ to 3′ which is not an order in which the plurality of fragments naturally occur in a nucleic acid. In a specific embodiment, all of the fragments in a shuffled nucleic acid molecule containing a plurality of nucleotide sequence fragments are from a single gene. In a more specific embodiment, the plurality of fragments originate from at least two different genes. In a more specific embodiment, the shuffled nucleic acid is operably linked to a promoter sequence. Another more specific embodiment is a use of a chimeric polynucleotide for modulating a characteristic in a plant cell, said chimeric polynucleotide including a promoter sequence operably linked to the shuffled nucleic acid. In a more specific embodiment, the shuffled nucleic acid is contained within a host cell.

In a further embodiment of the invention, the agent that can modulate the expression levels of a RING-like ubiquitin E3 ligase gene in a plant cell comprises:
(a) a polypeptide sequence as shown in any one of SEQ ID NOs: 2, 4, 6, 8, 10, or a functional fragment, domain, repeat, or chimera thereof;
(b) a polypeptide sequence having substantial similarity to (a);
(c) a polypeptide sequence encoded by a nucleotide sequence identical to or having substantial similarity to a nucleotide sequence listed in SEQ ID NOs:1, 3, 5, 7, 9, or a functional fragment or domain thereof, or a sequence complementary thereto; or
(d) a polypeptide sequence encoded by a nucleotide sequence capable of hybridizing under medium stringency conditions to a nucleotide sequence listed in SEQ ID NOs:1, 3, 5, 7, 9, or to a sequence complementary thereto.

In a more specific embodiment, the polypeptide contains a polypeptide sequence of any one of SEQ ID NOs: 2, 4, 6, 8, 10, or a fragment thereof. In a more specific embodiment, the polypeptide is a plant polypeptide. In a more specific embodiment, the plant is a dicot. In a more specific embodiment, the plant is a gymnosperm. In a more specific embodiment, the plant is a monocot. In a more specific embodiment, the monocot is a cereal. In a more specific embodiment, the cereal may be, for example, maize, wheat, barley, oats, rye, millet, sorghum, triticale, secale, einkorn, spelt, emmer, teff, miloflax, gramma grass, Tripsacum, and teosinte.

In one embodiment, the polypeptide is expressed throughout the plant. In a more specific embodiment, the polypeptide is expressed in a specific location or tissue of a plant. In a more specific embodiment, the location or tissue may be, for example, epidermis, root, vascular tissue, meristem, cambium, cortex, pith, leaf, and flower. In a most specific embodiment, the location or tissue is a seed.

In a specific embodiment, the polypeptide is useful for generating an antibody having immunoreactivity against a polypeptide encoded by a nucleotide sequence of SEQ ID NO:2, or fragment or domain thereof.

In a specific embodiment, a polypeptide having substantial similarity to a polypeptide sequence listed in SEQ ID NO:2, or exon or domain thereof, is an allelic variant of the polypeptide sequence listed in SEQ ID NO:2. In another specific embodiment, a polypeptide having substantial similarity to a polypeptide sequence listed in SEQ ID NO:2, or exon or domain thereof, is a naturally occurring variant of the polypeptide sequence listed in SEQ ID NO:2. In another specific embodiment, a polypeptide having substantial similarity to a polypeptide sequence listed in SEQ ID NO:2, or exon or domain thereof, is a polymorphic variant of the polypeptide sequence listed in SEQ ID NO:2.

In another specific embodiment, the polypeptide is a polypeptide sequence of SEQ ID NO:2. In another specific embodiment, the polypeptide is a functional fragment or domain. In yet another specific embodiment, the polypeptide is a chimera, where the chimera may include functional protein domains, including domains, repeats, post-translational modification sites, or other features. In a more specific embodiment, the polypeptide is a plant polypeptide. In a more specific embodiment, the plant is a dicot. In a more specific embodiment, the plant is a gymnosperm. In a more specific embodiment, the plant is a monocot. In a more specific embodiment, the monocot is a cereal. In a more specific embodiment, the cereal may be, for example, maize, wheat, barley, oats, rye, millet, sorghum, triticale, secale, einkorn, spelt, emmer, teff, milo, flax, gramma grass, Tripsacum, and teosinte.

In a specific embodiment, the polypeptide is expressed in a specific location or tissue of a plant. In a more specific embodiment, the location or tissue may be, for example, epidermis, root, vascular tissue, meristem, cambium, cortex, pith, leaf, and flower. In another specific embodiment, the location or tissue is a seed.

In a specific embodiment, the polypeptide sequence encoded by a nucleotide sequence having substantial similarity to a nucleotide sequence of SEQ ID NO:1 or a fragment or domain thereof or a sequence complementary thereto, includes a deletion or insertion of at least one nucleotide. In a more specific embodiment, the deletion or insertion is of less than about thirty nucleotides. In a most specific embodiment, the deletion or insertion is of less than about five nucleotides.

In a specific embodiment, the polypeptide sequence encoded by a nucleotide sequence having substantial similarity to a nucleotide sequence of SEQ ID NO:1, or a fragment or domain thereof or a sequence complementary thereto, includes a substitution of at least one codon. In a more specific embodiment, the substitution is conservative.

In a specific embodiment, the polypeptide sequences having substantial similarity to the polypeptide sequence of SEQ ID NO:2 or a fragment, domain, repeat, or chimeras thereof includes a deletion or insertion of at least one amino acid.

In a specific embodiment, the polypeptide sequences having substantial similarity to the polypeptide sequence of SEQ ID NO:2 or a fragment, domain, repeat, or chimeras thereof includes a substitution of at least one amino acid.

Embodiments of the present invention also contemplate a use of an expression cassette for modulating a characteristic in a plant cell including a promoter sequence operably linked to an isolated nucleic acid encoding a RING-like ubiquitin E3 ligase. In embodiments of the invention the isolated nucleic acid encoding a RING-like ubiquitin E3 ligase consists of or comprises:
(a) a nucleotide sequence of SEQ ID NOs:1, 3, 5, 7, 9, or a fragment or domain thereof;
(b) a nucleotide sequence encoding a polypeptide of any one of SEQ ID NOs: 2, 4, 6, 8, 10, a fragment or domain thereof;
(c) a nucleotide sequence having substantial similarity to (a) or (b);
(d) a nucleotide sequence capable of hybridizing to (a), (b) or (c);
(e) a nucleotide sequence complementary to (a), (b), (c) or (d); or
(f) a nucleotide sequence that is the reverse complement of (a), (b), (c) or (d).

Further encompassed within the invention is use of a recombinant vector for modulating a characteristic in a plant cell comprising an expression cassette including a promoter sequence operably linked to an isolated nucleic acid encoding a RING-like ubiquitin E3 ligase. In embodiments of the invention the recombinant vector comprises an isolated nucleic acid encoding a RING-like ubiquitin E3 ligase consists of or comprises:
(a) a nucleotide sequence of SEQ ID NOs:1, 3, 5, 7, 9, or a fragment or domain thereof;
(b) a nucleotide sequence encoding a polypeptide of any one of SEQ ID NOs: 2, 4, 6, 8, 10, a fragment or domain thereof;
(c) a nucleotide sequence having substantial similarity to (a) or (b);
(d) a nucleotide sequence capable of hybridizing to (a), (b) or (c)
(e) a nucleotide sequence complementary to (a), (b), (c) or (d); or
(f) a nucleotide sequence that is the reverse complement of (a), (b), (c) or (d).

Also encompassed are uses of plant cells, which contain expression cassettes, according to the present disclosure, and uses of plants, containing these plant cells.

Also encompassed are plant cells, which contain expression cassettes, according to the present disclosure, and plants, containing these plant cells. In a specific embodiment, the plant is a dicot. In a more specific embodiment, the dicot is selected from the group consisting of soybean, tobacco poplar or cotton. In another specific embodiment, the plant is a gymnosperm. In another specific embodiment, the plant is a monocot. In a more specific embodiment, the monocot is a cereal. In a more specific embodiment, the cereal may be, for example, maize, wheat, barley, oats, rye, millet, sorghum, triticale, secale, einkorn, spelt, emmer, teff, milo, flax, gramma grass, Tripsacum and teosinte.

In one embodiment, the expression cassette is expressed throughout the plant. In another embodiment, the expression cassette is expressed in a specific location or tissue of a plant. In a specific embodiment, the location or tissue may be, for example, epidermis, root, vascular tissue, meristem, cambium, cortex, pith, leaf, and flower. In an alternative specific embodiment, the location or tissue is a seed.

Embodiments of the present invention also provide the use of seed and isolated product from plants for modulating a characteristic in a plant cell, which contain an expression cassette including a promoter sequence operably linked to an isolated nucleic acid encoding a RING-like ubiquitin E3 ligase gene according to the present invention.

In a specific embodiment, the expression vector includes one or more elements such as, for example, but not limited to, a promoter-enhancer sequence, a selection marker sequence, an origin of replication, an epitope-tag encoding sequence, or an affinity purification-tag encoding sequence. In a more specific embodiment, the promoter-enhancer sequence may be, for example, the CaMV 35S promoter, the CaMV 19S promoter, the tobacco PR-1a promoter, ubiquitin and the phaseolin promoter. In another embodiment, the promoter is operable in plants, and more specifically, a constitutive or inducible promoter. In another specific embodiment, the selection marker sequence encodes an antibiotic resistance gene. In another specific embodiment, the epitope-tag sequence encodes V5, the peptide Phe-His-His-Thr-Thr, hemagglutinin, or glutathione-S-transferase. In another specific embodiment the affinity purification-tag sequence encodes a polyamino acid sequence or a polypeptide. In a more specific embodiment, the polyamino acid sequence is polyhistidine. In a more specific embodiment, the polypeptide is chitin binding domain or glutathione-S-transferase. In a more specific embodiment, the affinity purification-tag sequence comprises an intein encoding sequence.

In a specific embodiment, the expression vector is a eukaryotic expression vector or a prokaryotic expression vector. In a more specific embodiment, the eukaryotic expression vector includes a tissue-specific promoter. More specifically, the expression vector is operable in plants.

Embodiments of the present invention also relate to a plant modified by a method that includes introducing into a plant a nucleic acid where the nucleic acid is expressible in the plant in an amount effective to effect the modification. The modification can be an increase or decrease in the one or more traits of interest. The modification may include overexpression, underexpression, antisense modulation, sense suppression, inducible expression, inducible repression, or inducible modulation of a gene. In an embodiment of the invention the modification involved an increase or improvement in the trait of interest, for example, nitrogen utilization or yield.

In one embodiment, the expression cassette is involved in a function such as, for example, but not limited to, carbon, nitrogen and/or sulfur metabolism, nitrogen utilization, nitrogen assimilation, photosynthesis, lignin biosynthesis, anthocyanin biosynthesis, signal transduction, cell growth, reproduction, disease resistance, abiotic stress tolerance, nutritional composition, gene regulation, and/or differentiation. In a more specific embodiment, the expression cassette is involved in a function such as, nitrogen utilization, abiotic stress tolerance, enhanced yield, disease resistance and/or nutritional composition.

In one embodiment, the plant contains a modification to a phenotype or measurable characteristic of the plant, the modification being attributable to the expression of at least one gene contained in the expression cassette. In a specific embodiment, the modification may be, for example, carbon, nitrogen and/or sulfur metabolism, nitrogen utilization, nitrogen assimilation, photosynthesis, lignin biosynthesis, anthocyanin biosynthesis, signal transduction, cell growth, reproduction, disease resistance, abiotic stress tolerance, nutritional composition, gene regulation, and/or differentiation.

Embodiments of the present invention also provide seed and isolated products from plants which contain an expression cassette including a promoter sequence operably linked to an isolated nucleic acid containing a nucleotide sequence including:
(a) a nucleotide sequence of SEQ ID NOs:1, 3, 5, 7, 9, or a fragment or domain thereof,
(b) a nucleotide sequence encoding a polypeptide of any one of SEQ ID NOs: 2, 4, 6, 8, 10, a fragment or domain thereof;
(c) a nucleotide sequence having substantial similarity to (a) or (b);
(d) a nucleotide sequence capable of hybridizing to (a), (b) or (c)
(e) a nucleotide sequence complementary to (a), (b), (c) or (d); or
(f) a nucleotide sequence that is the reverse complement of (a), (b), (c) or (d) according to the present disclosure.

In a specific embodiment the isolated product includes an enzyme, a nutritional protein, a structural protein, an amino acid, a lipid, a fatty acid, a polysaccharide, a sugar, an alcohol, a fiber, a flavonoid, an alkaloid, a carotenoid, a propanoid, a steroid, a pigment, a vitamin and a plant hormone.

Embodiments of the present invention also relate to isolated products produced by expression of an isolated nucleic acid containing a nucleotide sequence including:
(a) a nucleotide sequence of SEQ ID NOs:1, 3, 5, 7, 9, or fragment or domain thereof;
(b) a nucleotide sequence encoding a polypeptide of any one of SEQ ID NOs: 2, 4, 6, 8, 10, or a fragment or domain thereof;
(c) a nucleotide sequence having substantial similarity to (a) or (b);
(d) a nucleotide sequence capable of hybridizing to (a) or (b);
(e) a nucleotide sequence complementary to (a), (b), (c) or (d); or
(f) a nucleotide sequence that is the reverse complement of (a),
(b) (c) or (d) according to the present disclosure.

In a specific embodiment, the product is produced in a plant. In another specific embodiment, the product is produced in cell culture. In another specific embodiment, the product is produced in a cell-free system. In another specific embodiment, the product includes an enzyme, a nutritional protein, a structural protein, an amino acid, a lipid, a fatty acid, a polysaccharide, a sugar, an alcohol, a fiber, a flavonoid, an alkaloid, a carotenoid, a propanoid, a steroid, a pigment, a vitamin and a plant hormone.

In one embodiment, the product is a RING-type ubiquitin E3 ligase. In a specific embodiment, the product is a polypeptide containing an amino acid sequence of any one of SEQ ID NOs: 2, 4, 6, 8, 10.

Embodiments of the present invention further relate to an isolated polynucleotide including a nucleotide sequence of at least 10 bases, which sequence is identical, complementary, or substantially similar to a region of any sequence of SEQ ID NOs:1, 3, 5, 7, 9, and wherein the polynucleotide is adapted for any of numerous uses.

In a specific embodiment, the polynucleotide is used as a chromosomal marker. In another specific embodiment, the polynucleotide is used as a marker for RFLP analysis. In another specific embodiment, the polynucleotide is used as a marker for quantitative trait linked breeding. In another specific embodiment, the polynucleotide is used as a marker for marker-assisted breeding. In another specific embodiment, the polynucleotide is used as a bait sequence in a two-hybrid system to identify sequences encoding polypeptides interacting with the polypeptide encoded by the bait sequence. In another specific embodiment, the polynucleotide is used as a diagnostic indicator for genotyping or identifying an individual or population of individuals. In another specific embodiment, the polynucleotide is used for genetic analysis to identify boundaries of genes or exons.

Embodiments of the present invention also relate to an expression vector comprising or consisting of a nucleic acid molecule including:
(a) a nucleic acid encoding a polypeptide as listed in any of SEQ ID NOs:2, 4, 6, 8, 10, or
(b) a fragment, one or more domains, or featured regions of any of SEQ ID NOs:1, 3, 5, 7, 9; or
(c) a complete nucleic acid sequence listed in any of SEQ ID NOs:1, 3, 5, 7, 9, or a fragment thereof, in combination with a heterologous sequence.

In a specific embodiment, the expression vector includes one or more elements such as, for example, but not limited to, a promoter-enhancer sequence, a selection marker sequence, an origin of replication, an epitope-tag encoding sequence, or an affinity purification-tag encoding sequence. In a more specific embodiment, the promoter-enhancer sequence may be, for example, the CaMV 35S promoter, the CaMV 19S promoter, the tobacco PR-1a promoter, ubiquitin and the phaseolin promoter. In another embodiment, the promoter is operable in plants, and more specifically, a constitutive or inducible promoter. In another specific embodiment, the selection marker sequence encodes an antibiotic resistance gene. In another specific embodiment, the epitope-tag sequence encodes V5, the peptide Phe-His-His-Thr-Thr, hemagglutinin, or glutathione-S-transferase. In another specific embodiment the affinity purification-tag sequence encodes a polyamino acid sequence or a polypeptide. In a more specific embodiment, the polyamino acid sequence is polyhistidine. In a more specific embodiment, the polypeptide is chitin binding domain or glutathione-S-transferase. In a more specific embodiment, the affinity purification-tag sequence comprises an intein encoding sequence.

In a specific embodiment, the expression vector is a eukaryotic expression vector or a prokaryotic expression vector. In a more specific embodiment, the eukaryotic expression vector includes a tissue-specific promoter. More specifically, the expression vector is operable in plants.

Embodiments of the present invention also relate to a cell comprising or consisting of a nucleic acid construct comprising an expression vector and a nucleic acid including a nucleic acid encoding a polypeptide as listed in any one of SEQ ID NOs: 2, 4, 6, 8, 10, or a nucleic acid sequence listed in any one of SEQ ID NOs:1, 3, 5, 7, 9, or a segment thereof, in combination with a heterologous sequence.

In a specific embodiment, the cell is a bacterial cell, a fungal cell, a plant cell, or an animal cell. In a specific embodiment, the cell is a plant cell. In a more specific embodiment, the polypeptide is expressed in a specific location or tissue of a plant. In a most specific embodiment, the location or tissue may be, for example, epidermis, root, vascular tissue, meristem, cambium, cortex, pith, leaf, and flower. In an alternate most specific embodiment, the location or tissue is a seed. In a specific embodiment, the polypeptide is involved in a function such as, for example, carbon, nitrogen and/or sulfur metabolism, nitrogen utilization, nitrogen assimilation, photosynthesis, lignin biosynthesis, anthocyanin biosynthesis, signal transduction, cell growth, reproduction, disease resistance, abiotic stress tolerance, nutritional composition, gene regulation, and/or differentiation.

Embodiments of the present invention contemplate a polypeptide containing a polypeptide sequence encoded by an isolated polynucleotide containing a nucleotide sequence of at least 10 bases, which sequence is identical, complementary, or substantially similar to a region of any of sequences of SEQ ID NOs:1, 3, 5, 7, 9, or functional fragment thereof and wherein the polynucleotide is adapted for a use including:
(a) use as a chromosomal marker to identify the location of the corresponding or complementary polynucleotide on a native or artificial chromosome;
(b) use as a marker for RFLP analysis;
(c) use as a marker for quantitative trait linked breeding;
(d) use as a marker for marker-assisted breeding;
(e) use as a bait sequence in a two-hybrid system to identify sequences encoding polypeptides interacting with the polypeptide encoded by the bait sequence;
(f) use as a diagnostic indicator for genotyping or identifying an individual or population of individuals; or
(g) use for genetic analysis to identify boundaries of genes or exons.

The invention also contemplates a use of an nucleic acid molecule comprising a nucleotide sequence of at least 10 bases, which sequence is identical, complementary, or substantially similar to a region of SEQ ID NO:3, or a functional fragment thereof and wherein the use is selected from the group consisting of:

(i) use as a marker for low nitrogen limitation adaptability;

(ii) use as a marker for increased lignin biosynthesis; or

(iii) use as a marker for low yield.

Also contemplated is a method of producing a plant comprising a modification thereto, including the steps of: (1) providing a nucleic acid which is an isolated nucleic acid containing a nucleotide sequence including:
(a) a nucleotide sequence listed as SEQ ID NOs:1, 3, 5, 7, 9, or exon or domain thereof;
(b) a nucleotide sequence having substantial similarity to (a);
(c) a nucleotide sequence capable of hybridizing to (a);
(d) a nucleotide sequence complementary to (a), (b) or (c); or
(e) a nucleotide sequence which is the reverse complement of (a), (b) or (c);
   and (2) introducing the nucleic acid into the plant, wherein the nucleic acid is expressible in the plant in an amount effective to effect the modification. In one embodiment, the modification comprises an altered characteristic in the plant, wherein the characteristic corresponds to the nucleic acid introduced into the plant. In other specific embodiments the characteristic corresponds to carbon, nitrogen and/or sulfur metabolism, nitrogen utilization, nitrogen assimilation, photosynthesis, lignin biosynthesis, anthocyanin biosynthesis, signal transduction, cell growth, reproduction, disease resistance, abiotic stress tolerance, nutritional composition, gene regulation, and/or differentiation.

In another embodiment, the modification includes an increased or decreased expression or accumulation of a product of the plant. Specifically, the product is a natural product of the plant. Equally specifically, the product is a new or altered product of the plant. Specifically, the product comprises a RING-like ubiquitin E3 ligase.

Also encompassed within the presently disclosed invention is a method of producing a recombinant protein, comprising the steps of:
(a) growing recombinant cells comprising a nucleic acid construct under suitable growth conditions, the construct comprising an expression vector and a nucleic acid including: a nucleic acid encoding a protein as listed in SEQ ID NOs: 2, 4, 6, 8, 10, or a nucleic acid sequence listed in SEQ ID NOs:1, 3, 5, 7, 9, or segments thereof; and
(b) isolating from the recombinant cells the recombinant protein expressed thereby.

Embodiments of the present invention provide a method of producing a recombinant protein in which the expression vector includes one or more elements including a promoter-enhancer sequence, a selection marker sequence, an origin of replication, an epitope-tag encoding sequence, and an affinity purification-tag encoding sequence. In one specific embodiment, the nucleic acid construct includes an epitope-tag encoding sequence and the isolating step includes use of an antibody specific for the epitope-tag. In another specific embodiment, the nucleic acid construct contains a polyamino acid encoding sequence and the isolating step includes use of a resin comprising a polyamino acid binding substance, specifically where the polyamino acid is polyhistidine and the polyamino binding resin is nickel-charged agarose resin. In yet another specific embodiment, the nucleic acid construct contains a polypeptide encoding sequence and the isolating step includes the use of a resin containing a polypeptide binding substance, specifically where the polypeptide is a chitin binding domain and the resin contains chitin-sepharose.

Embodiments of the present invention also relate to a plant modified by a method that includes introducing into a plant a nucleic acid where the nucleic acid is expressible in the plant in an amount effective to effect the modification. The modification can be, for example, carbon, nitrogen and/or sulfur metabolism, nitrogen utilization, nitrogen assimilation, photosynthesis, lignin biosynthesis, anthocyanin biosynthesis, signal transduction, cell growth, reproduction, disease resistance, abiotic stress tolerance, nutritional composition, gene regulation, and/or differentiation. In one embodiment, the modified plant has increased or decreased resistance to an herbicide, a stress, or a pathogen. In another embodiment, the modified plant has enhanced or diminished requirement for light, water, nitrogen, or trace elements. In yet another embodiment, the modified plant is enriched for an essential amino acid as a proportion of a protein fraction of the plant. The protein fraction may be, for example, total seed protein, soluble protein, insoluble protein, water-extractable protein, and lipid-associated protein. In yet another embodiment, the modified plant has increased or decreased anthocyanin pigmentation. In yet another embodiment, the modified plant has increased or decreased accumulation of lignin. In yet another embodiment, the plant has increased sensitivity to conditions of limiting nitrogen in the soil. The modification may include overexpression, underexpression, antisense modulation, sense suppression, inducible expression, inducible repression, or inducible modulation of a gene.

The invention further relates to a seed from a modified plant or an isolated product of a modified plant, where the product may be an enzyme, a nutritional protein, a structural protein, an amino acid, a lipid, a fatty acid, a polysaccharide, a sugar, an alcohol, a fiber, a flavonoid, an alkaloid, a carotenoid, a propanoid, a steroid, a pigment, a vitamin and a plant hormone.

The above Summary of Invention lists several embodiments of the invention, and in many cases lists variations and permutations of these embodiments. The Summary is merely exemplary of the numerous and varied embodiments. Mention of one or more specific features of a given embodiment is likewise exemplary. Such embodiment can typically exist with or without the feature(s) mentioned; likewise, those features can be applied to other embodiments of the invention, whether listed in this Summary or not. To avoid excessive repetition, this Summary does not list or suggest all possible combinations of such features.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Further aspects, features and advantages of this invention will become apparent from the detailed description of the specific embodiments that follow. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 is a series of photographs illustrating the low nitrogen induced early senescence phenotype in lines mutant. Wild type (Columbia, Col) and lines plants were grown in LB2 soil with 1, 3, or 10 mM nitrate, respectively, for 18 days (A), 26 days (B) and 32 days (C), showing early senescence phenotype in the lines plants supplied with 1 or 3 mM nitrate. DAG: days after seed germination. Arrows indicate the dead siliques. (D) and (E) were cauline leaves and developing siliques from the lines (upper panel) and Col (lower panel) plants supplied with 3 mM nitrate, respectively. The arrow in (E) indicates the senescing silique tip. Supplying senescing lines plants with 15 mM nitrate stopped senescence progress in the lines plants initially grown with 1 mM (F) or 3 mM (G) nitrate.

FIG. 2 is a depiction of the map-based cloning of the LINES gene and complementation of lines mutant. (A) The position of LINES locus was defined by two flanking SSLP markers NF21B7 (12 recombinants) and NT7123 (6 recombinants) on the top arm of Chromosome I. Further mapping located LINES locus on the BAC clone F22D16 flanked by the SSLP marker 473993 (1 recombinant) and the CAPS marker SNP247 (1 recombinant). This region is approximately 62.3 kb and contained 21 annotated genes, among which DNA fragment deletion was only detected in the gene At1g02860. Following complementation test confirmed At1g02860 is LINES gene. (B) Supplied with 3 mM nitrate, wild type and three lines plants independently transformed with At1g02860 cDNA did not show early senescence phenotype when supplied with 3 mM nitrate, while the lines plant transformed with the empty vector pGEAD and lines itself displayed early and rapid senescence at 26 days after seed germination. (C) and (D) Detection of various versions of At1g02860 genomic DNA and cDNA in wild type, lines, and transgenic lines plants by PCR and RT-PCR, respectively.

FIG. 3 is a molecular analysis of LINES gene. (A) Predicted amino acid sequence of LINES protein (SEQ ID NO:4). The deleted residues in LINES mutation are underlined. (B) Scheme of LINES structure with SPX and RING domain. Most part of the RING domain is deleted in the truncated LINES. (C) Phylogenetic analysis of LINES orthologs which contain both SPX and RING domain.

FIG. 4 is a comparison of nitrogen acquisition and senescence process in lines and Col plants grown under limited nitrogen supply. (A) Total nitrogen content percentage (w/w) in shoots at 18 days after seed germination (DAG). The values are means±standard error (n=3). (B) Expression of two major nitrate transporter genes NRT1.1 and NRT2.1 in lines and Col roots at 20 DAG. (C) Senescence progress in rosette leaves from the lines and Col plants at 26 DAG (upper panel) and 32 DAG (lower panel). Photographs show representative leaves at each position in a rosette. (D) The expression pattern of SAG12, the senescence marker gene, in lines and Col plants.

FIG. 5 is an analysis of nitrogen and carbon metabolite contents as well as anthocyanin amounts in the lines and Col plants grown under limited nitrogen supply changed with senescence process. The assayed metabolites include (A) nitrate; (B) total amino acids; (C) proteins; (D) total nitrogen percentage (w/w); (E) glucose; (F) fructose; (G) sucrose; (H) anthocyanin; (I) chlorophyll. Bars represent mean values±standard deviation (n=3-6).

FIG. 6 is the expression of related genes in the lines and Col plants supplied with limited nitrogen altered with senescence progress. The analyzed genes include those involved in nitrogen assimilation (NR1, NR2, and GS2), photosynthesis (RBCS and CAB1), and anthocyanin synthesis (CHS). SGA12 expression was used as the indicator for the senescence progress.

### DEFINITIONS

For clarity, certain terms used in the specification are defined and presented as follows:

“Associated with/operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

A “chimeric construct” is a recombinant nucleic acid sequence in which a promoter or regulatory nucleic acid sequence is operatively linked to, or associated with, a nucleic acid sequence that codes for an mRNA or which is expressed as a protein, such that the regulatory nucleic acid sequence is able to regulate transcription or expression of the associated nucleic acid sequence. The regulatory nucleic acid sequence of the chimeric construct is not normally operatively linked to the associated nucleic acid sequence as found in nature.

A “co-factor” is a natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

A “coding sequence” is a nucleic acid sequence that is transcribed into RNA such as mRNA, rRNA, tRNA, snRNA, sense RNA or antisense RNA. Specifically the RNA is then translated in an organism to produce a protein.

Complementary: “complementary” refers to two nucleotide sequences that comprise antiparallel nucleotide sequences capable of pairing with one another upon formation of hydrogen bonds between the complementary base residues in the antiparallel nucleotide sequences.

Enzyme activity: means herein the ability of an enzyme to catalyze the conversion of a substrate into a product. A substrate for the enzyme comprises the natural substrate of the enzyme but also comprises analogues of the natural substrate, which can also be converted, by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

Expression Cassette: “Expression cassette” as used herein means a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue or organ or stage of development.

The term “functional fragment” as used herein in relation to a nucleic acid or protein sequence means a fragment or portion of the sequence that retains the function of the full length sequence.

Gene: the term “gene” is used broadly to refer to any segment of DNA associated with a biological function. Thus, genes include coding sequences and/or the regulatory sequences required for their expression. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

Heterologous/exogenous: The terms “heterologous” and “exogenous” when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA) sequence naturally associated with a host cell into which it is introduced.

Hybridization: The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

Inhibitor: a chemical substance that inactivates the enzymatic activity of a protein such as a biosynthetic enzyme, receptor, signal transduction protein, structural gene product, or transport protein. The term “herbicide” (or “herbicidal compound”) is used herein to define an inhibitor applied to a plant at any stage of development, whereby the herbicide inhibits the growth of the plant or kills the plant.

Interaction: quality or state of mutual action such that the effectiveness or toxicity of one protein or compound on another protein is inhibitory (antagonists) or enhancing (agonists).

A nucleic acid sequence is “isocoding with” a reference nucleic acid sequence when the nucleic acid sequence encodes a polypeptide having the same amino acid sequence as the polypeptide encoded by the reference nucleic acid sequence.

Isogenic: plants that are genetically identical, except that they may differ by the presence or absence of a heterologous DNA sequence.

Isolated: in the context of the present invention, an isolated DNA molecule or an isolated enzyme is a DNA molecule or enzyme that, by human intervention, exists apart from its native environment and is therefore not a product of nature. An isolated DNA molecule or enzyme may exist in a purified form or may exist in a non-native environment such as, for example, in a transgenic host cell.

Mature protein: protein from which the transit peptide, signal peptide, and/or propeptide portions have been removed.

Minimal Promoter: the smallest piece of a promoter, such as a TATA element, that can support any transcription. A minimal promoter typically has greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Native: refers to a gene that is present in the genome of an untransformed plant cell.

Naturally occurring: the term “naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

Nucleic acid: the term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). The terms “nucleic acid” or “nucleic acid sequence” may also be used interchangeably with gene, cDNA, and mRNA encoded by a gene.

“ORF” means open reading frame.

Percent identity: the phrases “percent identity” or “percent identical,” in the context of two nucleic acid or protein sequences, refers to two or more sequences or subsequences that have for example 60%, specifically 70%, more specifically 80%, still more specifically 90%, even more specifically 95%, and most specifically at least 99% nucleotide or amino acid residue identity, when compared and aligned for maximum correspondence, as measured using one of the following sequence comparison algorithms or by visual inspection. Specifically, the percent identity exists over a region of the sequences that is at least about 50 residues in length, more specifically over a region of at least about 100 residues, and most specifically the percent identity exists over at least about 150 residues. In an especially specific embodiment, the percent identity exists over the entire length of the coding regions.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nim.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more specifically less than about 0.01, and most specifically less than about 0.001.

Pre-protein: protein that is normally targeted to a cellular organelle, such as a chloroplast, and still comprises its native transit peptide.

Purified: the term “purified,” when applied to a nucleic acid or protein, denotes that the nucleic acid or protein is essentially free of other cellular components with which it is associated in the natural state. It is specifically in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least about 50% pure, more specifically at least about 85% pure, and most specifically at least about 99% pure.

Two nucleic acids are “recombined” when sequences from each of the two nucleic acids are combined in a progeny nucleic acid. Two sequences are “directly” recombined when both of the nucleic acids are substrates for recombination. Two sequences are “indirectly recombined” when the sequences are recombined using an intermediate such as a cross-over oligonucleotide. For indirect recombination, no more than one of the sequences is an actual substrate for recombination, and in some cases, neither sequence is a substrate for recombination.

“Regulatory elements” refer to sequences involved in controlling the expression of a nucleotide sequence. Regulatory elements comprise a promoter operatively linked to the nucleotide sequence of interest and termination signals. They also typically encompass sequences required for proper translation of the nucleotide sequence.

Significant Increase: an increase in enzymatic activity that is larger than the margin of error inherent in the measurement technique, specifically an increase by about 2-fold or greater of the activity of the wild-type enzyme in the presence of the inhibitor, more specifically an increase by about 5-fold or greater, and most specifically an increase by about 10-fold or greater.

Significantly less: means that the amount of a product of an enzymatic reaction is reduced by more than the margin of error inherent in the measurement technique, specifically a decrease by about 2-fold or greater of the activity of the wild-type enzyme in the absence of the inhibitor, more specifically an decrease by about 5-fold or greater, and most specifically an decrease by about 10-fold or greater.

Specific Binding/immunological Cross-Reactivity: An indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions. The phrase “specifically (or selectively) binds to an antibody,” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the nucleic acid sequences of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tm) for the specific sequence at a defined ionic strength and pH. Typically, under “stringent conditions” a probe will hybridize to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences of the present invention: a reference nucleotide sequence specifically hybridizes to the reference nucleotide sequence in 7% sodium dodecyl sulfate (SDS), 0.5 M NaPO4, 1 mM EDTA at 50° C. with washing in 2×SSC, 0.1% SDS at 50° C., more desirably in 7% sodium dodecyl sulfate (SDS), 0.5 M NaPO4, 1 mM EDTA at 50° C. with washing in 1×SSC, 0.1% SDS at 50° C., more desirably still in 7% sodium dodecyl sulfate (SDS), 0.5 M NaPO4, 1 mM EDTA at 50° C. with washing in 0.5×SSC, 0.1% SDS at 50° C., specifically in 7% sodium dodecyl sulfate (SDS), 0.5 M NaPO4, 1 mM EDTA at 50° C. with washing in 0.1×SSC, 0.1% SDS at 50° C., more specifically in 7% sodium dodecyl sulfate (SDS), 0.5 M NaPO4, 1 mM EDTA at 50° C. with washing in 0.1×SSC, 0.1% SDS at 65° C.

A “subsequence” refers to a sequence of nucleic acids or amino acids that comprise a part of a longer sequence of nucleic acids or amino acids (e.g., protein) respectively.

Substantial similarity: The term “substantial similarity” in the context of two nucleic acid or protein sequences, refers to two or more sequences or subsequences that are substantially similar, for example that have 50%, specifically 60%, more specifically 70%, even more specifically 80%, still more specifically 90%, further more specifically 95%, and most specifically 99% sequence identity.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Transformation: a process for introducing heterologous DNA into a plant cell, plant tissue, or plant. Transformed plant cells, plant tissue, or plants are understood to encompass not only the end product of a transformation process, but also transgenic progeny thereof.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

Viability: “viability” as used herein refers to a fitness parameter of a plant. Plants are assayed for their homozygous performance of plant development, indicating which proteins are essential for plant growth.

### DETAILED DESCRIPTION OF THE INVENTION

### I. General Description of Trait Functional Genomics

The goal of functional genomics is to identify genes controlling expression of organismal phenotypes, and employs a variety of methodologies, including but not limited to bioinformatics, gene expression studies, gene and gene product interactions, genetics, biochemistry and molecular genetics. For example, bioinformatics can assign function to a given gene by identifying genes in heterologous organisms with a high degree of similarity (homology) at the amino acid or nucleotide level. Expression of a gene at the mRNA or protein levels can assign function by linking expression of a gene to an environmental response, a developmental process or a genetic (mutational) or molecular genetic (gene overexpression or underexpression) perturbation. Expression of a gene at the mRNA level can be ascertained either alone (Northern analysis) or in concert with other genes (microarray analysis), whereas expression of a gene at the protein level can be ascertained either alone (native or denatured protein gel or immunoblot analysis) or in concert with other genes (proteomic analysis). Knowledge of protein/protein and protein/DNA interactions can assign function by identifying proteins and nucleic acid sequences acting together in the same biological process. Genetics can assign function to a gene by demonstrating that DNA lesions (mutations) in the gene have a quantifiable effect on the organism, including but not limited to: its development; hormone biosynthesis and response; growth and growth habit (plant architecture); mRNA expression profiles; protein expression profiles; ability to resist diseases; tolerance of abiotic stresses; ability to acquire nutrients; photosynthetic efficiency; altered primary and secondary metabolism; and the composition of various plant organs. Biochemistry can assign function by demonstrating that the protein encoded by the gene, typically when expressed in a heterologous organism, possesses a certain enzymatic activity, alone or in combination with other proteins. Molecular genetics can assign function by overexpressing or underexpressing the gene in the native plant or in heterologous organisms, and observing quantifiable effects as described in functional assignment by genetics above. In functional genomics, any or all of these approaches are utilized, often in concert, to assign genes to functions across any of a number of organismal phenotypes.

It is recognized by those skilled in the art that these different methodologies can each provide data as evidence for the function of a particular gene, and that such evidence is stronger with increasing amounts of data used for functional assignment: specifically from a single methodology, more specifically from two methodologies, and even more specifically from more than two methodologies. In addition, those skilled in the art are aware that different methodologies can differ in the strength of the evidence for the assignment of gene function. Typically, but not always, a datum of biochemical, genetic and molecular genetic evidence is considered stronger than a datum of bioinformatic or gene expression evidence. Finally, those skilled in the art recognize that, for different genes, a single datum from a single methodology can differ in terms of the strength of the evidence provided by each distinct datum for the assignment of the function of these different genes.

The objective of silvicultural species trait functional genomics is to identify trait genes, i.e. genes capable of conferring useful traits in forest plants. Such traits include, but are not limited to: enhanced yield, whether in quantity or quality; enhanced nutrient acquisition and enhanced metabolic efficiency; enhanced or altered nutrient composition of plant tissues used for construction, fiber or processing; enhanced utility for industrial processing; enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. The deployment of such identified trait genes by either transgenic or non-transgenic means could materially improve forest plants for the benefit of silviculure.

The objective of crop trait functional genomics is to identify crop trait genes, i.e. genes capable of conferring useful agronomic traits in crop plants. Such agronomic traits include, but are not limited to: enhanced yield, whether in quantity or quality; enhanced nutrient acquisition and enhanced metabolic efficiency; enhanced or altered nutrient composition of plant tissues used for food, feed, fiber or processing; enhanced utility for agricultural or industrial processing; enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. The deployment of such identified trait genes by either transgenic or non-transgenic means could materially improve crop plants for the benefit of agriculture.

Cereals are the most important crop plants on the planet, in terms of both human and animal consumption. Genomic synteny (conservation of gene order within large chromosomal segments) is observed in rice, maize, wheat, barley, rye, oats and other agriculturally important monocots, which facilitates the mapping and isolation of orthologous genes from diverse cereal species based on the sequence of a single cereal gene. Rice has the smallest (˜420 Mb) genome among the cereal grains, and has recently been a major focus of public and private genomic and EST sequencing efforts.

To identify crop trait genes in the rice [wheat] genome controlling [trait], genes from the rice draft genome sequence [wheat EST databases] were prioritized based on one or more functional genomic methodologies. For example, genome-wide expression studies of rice plants infected with rice blast fungus (Magnaporthe grisea) were used to prioritize candidate genes controlling disease resistance. Full-length and partial cDNAs of rice trait gene candidates could then be predicted based on analysis of the rice whole-genome sequence, and isolated by designing and using primers for PCR amplification using a commercially available PCR primer-picking program. Primers were used for PCR amplification of full-length or partial cDNAs from rice cDNA libraries or first-strand cDNA. cDNA clones resulting from either approach were used for the construction of vectors designed for altering expression of these genes in transgenic plants using plant molecular genetic methodologies, which are described in detail below. Alteration of plant phenotype through overexpression or underexpression of key trait genes in transgenic plants is a robust and established method for assigning functions to plant genes. Assays to identify transgenic plants with alterations in traits of interest are to be used to unambiguously assign the utility of these genes for the improvement of rice, and by extension, other cereals, either by transgenic or classical breeding methods.

II. Identifying, Cloning and Sequencing cDNAs

The cloning and sequencing of the cDNAs of the present invention are described in Example 1.

The isolated nucleic acids and proteins of the present invention are usable over a range of plants, gymnosperms, monocots and dicots, in particular monocots such as rice, wheat, barley and maize. In a more specific embodiment, the monocot is a cereal. In a more specific embodiment, the cereal may be, for example, maize, wheat, barley, oats, rye, millet, sorghum, triticale, secale, einkorn, spelt, emmer, teff, milo, flax, gramma grass, Tripsacum sp., or teosinte. In a most specific embodiment, the cereal is rice. Other plants genera include, but are not limited to, Cucurbita, Rosa, Vitis, Juglans, Gragaria, Lotus, Medicago, Onobrychis, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium, and Triticum.

The present invention also provides a method of genotyping a plant or plant part comprising a nucleic acid molecule of the present invention. Optionally, the plant is a monocot such as, but not limited rice or wheat. Genotyping provides a means of distinguishing homologs of a chromosome pair and can be used to differentiate segregants in a plant population. Molecular marker methods can be used in phylogenetic studies, characterizing genetic relationships among crop varieties, identifying crosses or somatic hybrids, localizing chromosomeal segments affecting mongenic traits, map based cloning, and the study of quantitative inheritance (see Plant Molecular Biology: A Laboratory Manual, Chapter 7, Clark ed., Springer-Verlag, Berlin 1997; Paterson, A. H., “The DNA Revolution”, chapter 2 in Genome Mapping in Plants, Paterson, A. H. ed., Academic Press/R.G. Lands Co., Austin, Tex. 1996).

The method of genotyping may employ any number of molecular marker analytical techniques such as, but not limited to, restriction length polymorphisms (RFLPs). As is well known in the art, RFLPs are produced by differences in the DNA restriction fragment lengths resulting from nucleotide differences between alleles of the same gene. Thus, the present invention provides a method of following segregation of a gene or nucleic acid of the present invention or chromosomal sequences genetically linked by using RFLP analysis. Linked chromosomal sequences are within 50 centiMorgans (50 cM), within 40 or 30 cM, specifically within 20 or 10 cM, more specifically within 5, 3, 2, or 1 cM of the nucleic acid of the invention.

### III. Traits of Interest

The present invention encompasses the identification and isolation of polynucleotides encoding proteins involved in nitrogen utilization, anthocyanin biosynthesis, and lignin biosynthesis. Altering the expression of genes related to these traits can be used to improve or modify plants, wood, and/or grain, as desired. Examples describe the isolated genes of interest and methods of analyzing the alteration of expression and their effects on the plant characteristics.

One aspect of the present invention provides compositions and methods for altering (i.e. increasing or decreasing) the level of nucleic acid molecules and polypeptides of the present invention in plants. In particular, the nucleic acid molecules and polypeptides of the invention are expressed constitutively, temporally or spatially, e.g. at developmental stages, in certain tissues, and/or quantities, which are uncharacteristic of non-recombinantly engineered plants. Therefore, the present invention provides utility in such exemplary applications as altering the specified characteristics identified above.

### VI. Controlling Gene Expression in Transgenic Plants

The invention further relates to transformed cells comprising the nucleic acid molecules, transformed plants, seeds, and plant parts, and methods of modifying phenotypic traits of interest by altering the expression of the genes of the invention.

### A. Modification of Coding Sequences and Adjacent Sequences

The transgenic expression in plants of genes derived from heterologous sources may involve the modification of those genes to achieve and optimize their expression in plants. In particular, bacterial ORFs which encode separate enzymes but which are encoded by the same transcript in the native microbe are best expressed in plants on separate transcripts. To achieve this, each microbial ORF is isolated individually and cloned within a cassette which provides a plant promoter sequence at the 5′ end of the ORF and a plant transcriptional terminator at the 3′ end of the ORF. The isolated ORF sequence specifically includes the initiating ATG codon and the terminating STOP codon but may include additional sequence beyond the initiating ATG and the STOP codon. In addition, the ORF may be truncated, but still retain the required activity; for particularly long ORFs, truncated versions which retain activity may be preferable for expression in transgenic organisms. By “plant promoter” and “plant transcriptional terminator” it is intended to mean promoters and transcriptional terminators that operate within plant cells. This includes promoters and transcription terminators that may be derived from non-plant sources such as viruses (an example is the Cauliflower Mosaic Virus).

In some cases, modification to the ORF coding sequences and adjacent sequence is not required. It is sufficient to isolate a fragment containing the ORF of interest and to insert it downstream of a plant promoter. For example, Gaffney et al. (Science 261: 754-756 (1993)) have expressed the Pseudomonas nahG gene in transgenic plants under the control of the CaMV 35S promoter and the CaMV tml terminator successfully without modification of the coding sequence and with nucleotides of the Pseudomonas gene upstream of the ATG still attached, and nucleotides downstream of the STOP codon still attached to the nahG ORF. Specifically, as little adjacent microbial sequence as possible should be left attached upstream of the ATG and downstream of the STOP codon. In practice, such construction may depend on the availability of restriction sites.

In other cases, the expression of genes derived from microbial sources may provide problems in expression. These problems have been well characterized in the art and are particularly common with genes derived from certain sources such as Bacillus. These problems may apply to the nucleotide sequence of this invention and the modification of these genes can be undertaken using techniques now well known in the art. The following problems may be encountered:

1. Codon Usage.

The specific codon usage in plants differs from the specific codon usage in certain microorganisms. Comparison of the usage of codons within a cloned microbial ORF to usage in plant genes (and in particular genes from the target plant) will enable an identification of the codons within the ORF that should specifically be changed. Typically plant evolution has tended towards a strong preference of the nucleotides C and G in the third base position of monocotyledons, whereas dicotyledons often use the nucleotides A or T at this position. By modifying a gene to incorporate specific codon usage for a particular target transgenic species, many of the problems described below for GC/AT content and illegitimate splicing will be overcome.

2. GC/AT Content.

Plant genes typically have a GC content of more than 35%. ORF sequences which are rich in A and T nucleotides can cause several problems in plants. Firstly, motifs of ATTTA are believed to cause destabilization of messages and are found at the 3′ end of many short-lived mRNAs. Secondly, the occurrence of polyadenylation signals such as AATAAA at inappropriate positions within the message is believed to cause premature truncation of transcription. In addition, monocotyledons may recognize AT-rich sequences as splice sites (see below).

3. Sequences Adjacent to the Initiating Methionine.

Plants differ from microorganisms in that their messages do not possess a defined ribosome-binding site. Rather, it is believed that ribosomes attach to the 5′ end of the message and scan for the first available ATG at which to start translation. Nevertheless, it is believed that there is a preference for certain nucleotides adjacent to the ATG and that expression of microbial genes can be enhanced by the inclusion of a eukaryotic consensus translation initiator at the ATG. Clontech (1993/1994 catalog, page 210, incorporated herein by reference) have suggested one sequence as a consensus translation initiator for the expression of the E. coli uidA gene in plants. Further, Joshi (N.A.R. 15: 6643-6653 (1987), incorporated herein by reference) has compared many plant sequences adjacent to the ATG and suggests another consensus sequence. In situations where difficulties are encountered in the expression of microbial ORFs in plants, inclusion of one of these sequences at the initiating ATG may improve translation. In such cases the last three nucleotides of the consensus may not be appropriate for inclusion in the modified sequence due to their modification of the second AA residue. Specific sequences adjacent to the initiating methionine may differ between different plant species. A survey of 14 maize genes located in the GenBank database provided the following results:

### Position Before the Initiating ATG in 14 Maize Genes:

| | −10 | −9 | −8 | −7 | −6 | −5 | −4 | −3 | −2 | −1 |
|---|---|---|---|---|---|---|---|---|---|---|
| C | 3 | 8 | 4 | 6 | 2 | 5 | 6 | 0 | 10 | 7 |
| T | 3 | 0 | 3 | 4 | 3 | 2 | 1 | 1 | 1 | 0 |
| A | 2 | 3 | 1 | 4 | 3 | 2 | 3 | 7 | 2 | 3 |
| G | 6 | 3 | 6 | 0 | 6 | 5 | 4 | 6 | 1 | 5 |

This analysis can be done for the desired plant species into which the nucleotide sequence is being incorporated, and the sequence adjacent to the ATG modified to incorporate the specific nucleotides.

4. Removal of Illegitimate Splice Sites.

Genes cloned from non-plant sources and not optimized for expression in plants may also contain motifs which may be recognized in plants as 5′ or 3′ splice sites, and be cleaved, thus generating truncated or deleted messages. These sites can be removed using the techniques well known in the art.

Techniques for the modification of coding sequences and adjacent sequences are well known in the art. In cases where the initial expression of a microbial ORF is low and it is deemed appropriate to make alterations to the sequence as described above, then the construction of synthetic genes can be accomplished according to methods well known in the art. These are, for example, described in the published patent disclosures EP 0 385 962 (to Monsanto), EP 0 359 472 (to Lubrizol) and WO 93/07278 (to Ciba-Geigy), all of which are incorporated herein by reference. In most cases it is preferable to assay the expression of gene constructions using transient assay protocols (which are well known in the art) prior to their transfer to transgenic plants.

### B. Construction of Plant Expression Cassettes

Coding sequences intended for expression in transgenic plants are first assembled in expression cassettes behind a suitable promoter expressible in plants. The expression cassettes may also comprise any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors described below. The following is a description of various components of typical expression cassettes.

1. Promoters

The selection of the promoter used in expression cassettes will determine the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters can be used, including the gene's native promoter. The following are non-limiting examples of promoters that may be used in expression cassettes.

a. Constitutive Expression, the Ubiquitin Promoter:

Ubiquitin is a gene product known to accumulate in many cell types and its promoter has been cloned from several species for use in transgenic plants (e.g. sunflower—Binet et al. Plant Science 79: 87-94 (1991); maize—Christensen et al. Plant Molec. Biol. 12: 619-632 (1989); and Arabidopsis—Callis et al., J. Biol. Chem. 265:12486-12493 (1990) and Norris et al., Plant Mol. Biol. 21:895-906 (1993)). The maize ubiquitin promoter has been developed in transgenic monocot systems and its sequence and vectors constructed for monocot transformation are disclosed in the patent publication EP 0 342 926 (to Lubrizol) which is herein incorporated by reference. Taylor et al. (Plant Cell Rep. 12: 491-495 (1993)) describe a vector (pAHC25) that comprises the maize ubiquitin promoter and first intron and its high activity in cell suspensions of numerous monocotyledons when introduced via microprojectile bombardment. The Arabidopsis ubiquitin promoter is ideal for use with the nucleotide sequences of the present invention. The ubiquitin promoter is suitable for gene expression in transgenic plants, both monocotyledons and dicotyledons. Suitable vectors are derivatives of pAHC25 or any of the transformation vectors described in this application, modified by the introduction of the appropriate ubiquitin promoter and/or intron sequences.

b. Constitutive Expression, the CaMV 35S Promoter:

Construction of the plasmid pCGN1761 is described in the published patent application EP 0 392 225 (Example 23), which is hereby incorporated by reference. pCGN1761 contains the “double” CaMV 35S promoter and the tml transcriptional terminator with a unique EcoRI site between the promoter and the terminator and has a pUC-type backbone. A derivative of pCGN1761 is constructed which has a modified polylinker which includes NotI and XhoI sites in addition to the existing EcoRI site. This derivative is designated pCGN1761ENX. pCGN1761ENX is useful for the cloning of cDNA sequences or coding sequences (including microbial ORF sequences) within its polylinker for the purpose of their expression under the control of the 35S promoter in transgenic plants. The entire 35S promoter-coding sequence-tml terminator cassette of such a construction can be excised by HindIII, SphI, SalI, and XbaI sites 5′ to the promoter and XbaI, BamHI and BglI sites 3′ to the terminator for transfer to transformation vectors such as those described below. Furthermore, the double 35S promoter fragment can be removed by 5′ excision with HindIII, SphI, SalI, XbaI, or PstI, and 3′ excision with any of the polylinker restriction sites (EcoRI, NotI or XhoI) for replacement with another promoter. If desired, modifications around the cloning sites can be made by the introduction of sequences that may enhance translation. This is particularly useful when overexpression is desired. For example, pCGN1761ENX may be modified by optimization of the translational initiation site as described in Example 37 of U.S. Pat. No. 5,639,949, incorporated herein by reference.

c. Constitutive Expression, the Actin Promoter:

Several isoforms of actin are known to be expressed in most cell types and consequently the actin promoter is a good choice for a constitutive promoter. In particular, the promoter from the rice ActI gene has been cloned and characterized (McElroy et al. Plant Cell 2: 163-171 (1990)). A 1.3 kb fragment of the promoter was found to contain all the regulatory elements required for expression in rice protoplasts. Furthermore, numerous expression vectors based on the ActI promoter have been constructed specifically for use in monocotyledons (McElroy et al. Mol. Gen. Genet. 231: 150-160 (1991)). These incorporate the ActI-intron 1, AdhI 5′ flanking sequence and AdhI-intron 1 (from the maize alcohol dehydrogenase gene) and sequence from the CaMV 35S promoter. Vectors showing highest expression were fusions of 35S and ActI intron or the ActI 5′ flanking sequence and the ActI intron. Optimization of sequences around the initiating ATG (of the GUS reporter gene) also enhanced expression. The promoter expression cassettes described by McElroy et al. (Mol. Gen. Genet. 231: 150-160 (1991)) can be easily modified for gene expression and are particularly suitable for use in monocotyledonous hosts. For example, promoter-containing fragments is removed from the McElroy constructions and used to replace the double 35S promoter in pCGN1761ENX, which is then available for the insertion of specific gene sequences. The fusion genes thus constructed can then be transferred to appropriate transformation vectors. In a separate report, the rice ActI promoter with its first intron has also been found to direct high expression in cultured barley cells (Chibbar et al. Plant Cell Rep. 12: 506-509 (1993)).

d. Inducible Expression, PR-1 Promoters:

The double 35S promoter in pCGN1761ENX may be replaced with any other promoter of choice that will result in suitably high expression levels. By way of example, one of the chemically regulatable promoters described in U.S. Pat. No. 5,614,395, such as the tobacco PR-1a promoter, may replace the double 35S promoter. Alternately, the Arabidopsis PR-1 promoter described in Lebel et al., Plant J. 16:223-233 (1998) may be used. The promoter of choice is specifically excised from its source by restriction enzymes, but can alternatively be PCR-amplified using primers that carry appropriate terminal restriction sites. Should PCR-amplification be undertaken, the promoter should be re-sequenced to check for amplification errors after the cloning of the amplified promoter in the target vector. The chemically/pathogen regulatable tobacco PR-1a promoter is cleaved from plasmid pCIB1004 (for construction, see example 21 of EP 0 332 104, which is hereby incorporated by reference) and transferred to plasmid pCGN1761ENX (Uknes et al., Plant Cell 4: 645-656 (1992)). pCIB1004 is cleaved with NcoI and the resultant 3′ overhang of the linearized fragment is rendered blunt by treatment with T4 DNA polymerase. The fragment is then cleaved with HindIII and the resultant PR-1a promoter-containing fragment is gel purified and cloned into pCGN1761ENX from which the double 35S promoter has been removed. This is accomplished by cleavage with XhoI and blunting with T4 polymerase, followed by cleavage with HindIII, and isolation of the larger vector-terminator containing fragment into which the pCIB1004 promoter fragment is cloned. This generates a pCGN1761ENX derivative with the PR-1a promoter and the tml terminator and an intervening polylinker with unique EcoRI and NotI sites. The selected coding sequence can be inserted into this vector, and the fusion products (i.e. promoter-gene-terminator) can subsequently be transferred to any selected transformation vector, including those described infra. Various chemical regulators may be employed to induce expression of the selected coding sequence in the plants transformed according to the present invention, including the benzothiadiazole, isonicotinic acid, and salicylic acid compounds disclosed in U.S. Pat. Nos. 5,523,311 and 5,614,395.

e. Inducible Expression, an Ethanol-Inducible Promoter:

A promoter inducible by certain alcohols or ketones, such as ethanol, may also be used to confer inducible expression of a coding sequence of the present invention. Such a promoter is for example the alcA gene promoter from Aspergillus nidulans (Caddick et al. (1998) Nat. Biotechnol 16:177-180). In A. nidulans, the alcA gene encodes alcohol dehydrogenase 1, the expression of which is regulated by the AlcR transcription factors in presence of the chemical inducer. For the purposes of the present invention, the CAT coding sequences in plasmid palcA:CAT comprising a alcA gene promoter sequence fused to a minimal 35S promoter (Caddick et al. (1998) Nat. Biotechnol 16:177-180) are replaced by a coding sequence of the present invention to form an expression cassette having the coding sequence under the control of the alcA gene promoter. This is carried out using methods well known in the art.

f. Inducible Expression, a Glucocorticoid-Inducible Promoter:

Induction of expression of a nucleic acid sequence of the present invention using systems based on steroid hormones is also contemplated. For example, a glucocorticoid-mediated induction system is used (Aoyama and Chua (1997) The Plant Journal 11: 605-612) and gene expression is induced by application of a glucocorticoid, for example a synthetic glucocorticoid, specifically dexamethasone, specifically at a concentration ranging from 0.1 mM to 1 mM, more specifically from 10 mM to 100 mM. For the purposes of the present invention, the luciferase gene sequences are replaced by a nucleic acid sequence of the invention to form an expression cassette having a nucleic acid sequence of the invention under the control of six copies of the GAL4 upstream activating sequences fused to the 35S minimal promoter. This is carried out using methods well known in the art. The trans-acting factor comprises the GAL4 DNA-binding domain (Keegan et al. (1986) Science 231: 699-704) fused to the transactivating domain of the herpes viral protein VP16 (Triezenberg et al. (1988) Genes Devel. 2: 718-729) fused to the hormone-binding domain of the rat glucocorticoid receptor (Picard et al. (1988) Cell 54: 1073-1080). The expression of the fusion protein is controlled either by a promoter known in the art or described here. This expression cassette is also comprised in the plant comprising a nucleic acid sequence of the invention fused to the 6×GAL4/minimal promoter. Thus, tissue- or organ-specificity of the fusion protein is achieved leading to inducible tissue- or organ-specificity of the insecticidal toxin.

g. Root Specific Expression:

Another pattern of gene expression is root expression. A suitable root promoter is the promoter of the maize metallothionein-like (MTL) gene described by de Framond (FEBS 290: 103-106 (1991)) and also in U.S. Pat. No. 5,466,785, incorporated herein by reference. This “MTL” promoter is transferred to a suitable vector such as pCGN1761ENX for the insertion of a selected gene and subsequent transfer of the entire promoter-gene-terminator cassette to a transformation vector of interest.

h. Wound-Inducible Promoters:

Wound-inducible promoters may also be suitable for gene expression. Numerous such promoters have been described (e.g. Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), Warner et al. Plant J. 3: 191-201 (1993)) and all are suitable for use with the instant invention. Logemann et al. describe the 5′ upstream sequences of the dicotyledonous potato wunl gene. Xu et al. show that a wound-inducible promoter from the dicotyledon potato (pin2) is active in the monocotyledon rice. Further, Rohrmeier & Lehle describe the cloning of the maize Wipl cDNA which is wound induced and which can be used to isolate the cognate promoter using standard techniques. Similar, Firek et al. and Warner et al. have described a wound-induced gene from the monocotyledon Asparagus officinalis, which is expressed at local wound and pathogen invasion sites. Using cloning techniques well known in the art, these promoters can be transferred to suitable vectors, fused to the genes pertaining to this invention, and used to express these genes at the sites of plant wounding.

i. Pith-Specific Expression:

Patent Application WO 93/07278, which is herein incorporated by reference, describes the isolation of the maize trpA gene, which is preferentially expressed in pith cells. The gene sequence and promoter extending up to −1726 bp from the start of transcription are presented. Using standard molecular biological techniques, this promoter, or parts thereof, can be transferred to a vector such as pCGN1761 where it can replace the 35S promoter and be used to drive the expression of a foreign gene in a pith-specific manner. In fact, fragments containing the pith-specific promoter or parts thereof can be transferred to any vector and modified for utility in transgenic plants.

j. Leaf-Specific Expression:

A maize gene encoding phosphoenol carboxylase (PEPC) has been described by Hudspeth & Grula (Plant Molec Biol 12: 579-589 (1989)). Using standard molecular biological techniques the promoter for this gene can be used to drive the expression of any gene in a leaf-specific manner in transgenic plants.

k. Pollen-Specific Expression:

WO 93/07278 describes the isolation of the maize calcium-dependent protein kinase (CDPK) gene which is expressed in pollen cells. The gene sequence and promoter extend up to 1400 bp from the start of transcription. Using standard molecular biological techniques, this promoter or parts thereof, can be transferred to a vector such as pCGN1761 where it can replace the 35S promoter and be used to drive the expression of a nucleic acid sequence of the invention in a pollen-specific manner.

2. Transcriptional Terminators

A variety of transcriptional terminators are available for use in expression cassettes. These are responsible for the termination of transcription beyond the transgene and correct mRNA polyadenylation. Appropriate transcriptional terminators are those that are known to function in plants and include the CaMV 35S terminator, the tml terminator, the nopaline synthase terminator and the pea rbcS E9 terminator. These can be used in both monocotyledons and dicotyledons. In addition, a gene's native transcription terminator may be used.

3. Sequences for the Enhancement or Regulation of Expression

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronzel gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picornavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Sarnow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

In addition to incorporating one or more of the aforementioned elements into the 5′ regulatory region of a target expression cassette of the invention, other elements peculiar to the target expression cassette may also be incorporated. Such elements include but are not limited to a minimal promoter. By minimal promoter it is intended that the basal promoter elements are inactive or nearly so without upstream activation. Such a promoter has low background activity in plants when there is no transactivator present or when enhancer or response element binding sites are absent. One minimal promoter that is particularly useful for target genes in plants is the Bz1 minimal promoter, which is obtained from the bronzel gene of maize. The Bz1 core promoter is obtained from the “myc” mutant Bz1-luciferase construct pBz1LucR98 via cleavage at the NheI site located at −53 to −58. Roth et al., Plant Cell 3: 317 (1991). The derived Bz1 core promoter fragment thus extends from −53 to +227 and includes the Bz1 intron-1 in the 5′ untranslated region. Also useful for the invention is a minimal promoter created by use of a synthetic TATA element. The TATA element allows recognition of the promoter by RNA polymerase factors and confers a basal level of gene expression in the absence of activation (see generally, Mukumoto (1993) Plant Mol Biol 23: 995-1003; Green (2000) Trends Biochem Sci 25: 59-63)

4. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). These signal sequences can be fused to heterologous gene products to effect the import of heterologous products into the chloroplast (van den Broeck, et al. Nature 313: 358-363 (1985)). DNA encoding for appropriate signal sequences can be isolated from the 5′ end of the cDNAs encoding the RUBISCO protein, the CAB protein, the EPSP synthase enzyme, the GS2 protein and many other proteins which are known to be chloroplast localized. See also, the section entitled “Expression With Chloroplast Targeting” in Example 37 of U.S. Pat. No. 5,639,949.

Other gene products are localized to other organelles such as the mitochondrion and the peroxisome (e.g. Unger et al. Plant Molec. Biol. 13: 411-418 (1989)). The cDNAs encoding these products can also be manipulated to effect the targeting of heterologous gene products to these organelles. Examples of such sequences are the nuclear-encoded ATPases and specific aspartate amino transferase isoforms for mitochondria. Targeting cellular protein bodies has been described by Rogers et al. (Proc. Natl. Acad. Sci. USA 82: 6512-6516 (1985)).

In addition, sequences have been characterized which cause the targeting of gene products to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)).

By the fusion of the appropriate targeting sequences described above to transgene sequences of interest it is possible to direct the transgene product to any organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected should include the known cleavage site, and the fusion constructed should take into account any amino acids after the cleavage site which are required for cleavage. In some cases this requirement may be fulfilled by the addition of a small number of amino acids between the cleavage site and the transgene ATG or, alternatively, replacement of some amino acids within the transgene sequence. Fusions constructed for chloroplast import can be tested for efficacy of chloroplast uptake by in vitro translation of in vitro transcribed constructions followed by in vitro chloroplast uptake using techniques described by Bartlett et al. In: Edelmann et al. (Eds.) Methods in Chloroplast Molecular Biology, Elsevier pp 1081-1091 (1982) and Wasmann et al. Mol. Gen. Genet. 205: 446-453 (1986). These construction techniques are well known in the art and are equally applicable to mitochondria and peroxisomes.

The above-described mechanisms for cellular targeting can be utilized not only in conjunction with their cognate promoters, but also in conjunction with heterologous promoters so as to effect a specific cell-targeting goal under the transcriptional regulation of a promoter that has an expression pattern different to that of the promoter from which the targeting signal derives.

### C. Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the specific transformation technique and the target species for transformation. For certain target species, different antibiotic or herbicide selection markers may be specific. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet. 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)). Below, the construction of two typical vectors suitable for Agrobacterium transformation is described.

a. pCIB200 and pCIB2001:

The binary vectors pCIB200 and pCIB2001 are used for the construction of recombinant vectors for use with Agrobacterium and are constructed in the following manner. pTJS75kan is created by NarI digestion of pTJS75 (Schmidhauser & Helinski, J. Bacteriol. 164: 446-455 (1985)) allowing excision of the tetracycline-resistance gene, followed by insertion of an AccI fragment from pUC4K carrying an NPTII (Messing & Vierra, Gene 19: 259-268 (1982): Bevan et al., Nature 304: 184-187 (1983): McBride et al., Plant Molecular Biology 14: 266-276 (1990)). XhoI linkers are ligated to the EcoRV fragment of PCIB7 which contains the left and right T-DNA borders, a plant selectable nos/nptII chimeric gene and the pUC polylinker (Rothstein et al., Gene 53: 153-161 (1987)), and the XhoI-digested fragment are cloned into SalI-digested pTJS75kan to create pCIB200 (see also EP 0 332 104, example 19). pCIB200 contains the following unique polylinker restriction sites: EcoRI, SstI, KpnI, BglII, XbaI, and SalI. pCIB2001 is a derivative of pCIB200 created by the insertion into the polylinker of additional restriction sites. Unique restriction sites in the polylinker of pCIB2001 are EcoRI, SstI, KpnI, BglII, XbaI, SalI, MluI, BclI, AvrII, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

b. pCIB10 and Hygromycin Selection Derivatives thereof:

The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et al. (Gene 53: 153-161 (1987)). Various derivatives of pCIB10 are constructed which incorporate the gene for hygromycin B phosphotransferase described by Gritz et al. (Gene 25: 179-188 (1983)). These derivatives enable selection of transgenic plant cells on hygromycin only (pCIB743), or hygromycin and kanamycin (pCIB715, pCIB717).

2. Vectors Suitable for Non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the specific selection for the species being transformed. Below, the construction of typical vectors suitable for non-Agrobacterium transformation is described.

a. pCIB3064:

pCIB3064 is a pUC-derived vector suitable for direct gene transfer techniques in combination with selection by the herbicide basta (or phosphinothricin). The plasmid pCIB246 comprises the CaMV 35S promoter in operational fusion to the E. coli GUS gene and the CaMV 35S transcriptional terminator and is described in the PCT published application WO 93/07278. The 35S promoter of this vector contains two ATG sequences 5′ of the start site. These sites are mutated using standard PCR techniques in such a way as to remove the ATGs and generate the restriction sites SspI and PvuII. The new restriction sites are 96 and 37 bp away from the unique SalI site and 101 and 42 bp away from the actual start site. The resultant derivative of pCIB246 is designated pCIB3025. The GUS gene is then excised from pCIB3025 by digestion with SalI and SacI, the termini rendered blunt and religated to generate plasmid pCIB3060. The plasmid pJIT82 is obtained from the John Innes Centre, Norwich and the a 400 bp SmaI fragment containing the bar gene from Streptomyces viridochromogenes is excised and inserted into the HpaI site of pCIB3060 (Thompson et al. EMBO J. 6: 2519-2523 (1987)). This generated pCIB3064, which comprises the bar gene under the control of the CaMV 35S promoter and terminator for herbicide selection, a gene for ampicillin resistance (for selection in E. coli) and a polylinker with the unique sites SphI, PstI, HindIII, and BamHI. This vector is suitable for the cloning of plant expression cassettes containing their own regulatory signals.

b. pSOG19 and pSOG35:

pSOG35 is a transformation vector that utilizes the E. coli gene dihydrofolate reductase (DFR) as a selectable marker conferring resistance to methotrexate. PCR is used to amplify the 35S promoter (−800 bp), intron 6 from the maize AdhI gene (−550 bp) and 18 bp of the GUS untranslated leader sequence from pSOG10. A 250-bp fragment encoding the E. coli dihydrofolate reductase type II gene is also amplified by PCR and these two PCR fragments are assembled with a SacI-PstI fragment from pB1221 (Clontech) which comprises the pUC19 vector backbone and the nopaline synthase terminator. Assembly of these fragments generates pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generates the vector pSOG35. pSOG19 and pSOG35 carry the pUC gene for ampicillin resistance and have HindIII, SphI, PstI and EcoRI sites available for the cloning of foreign substances.

3. Vector Suitable for Chloroplast Transformation

For expression of a nucleotide sequence of the present invention in plant plastids, plastid transformation vector pPH143 (WO 97/32011, example 36) is used. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

### D. Transformation

Once a nucleic acid sequence of the invention has been cloned into an expression system, it is transformed into a plant cell. The receptor and target expression cassettes of the present invention can be introduced into the plant cell in a number of art-recognized ways. Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

1. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a specific technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Höfgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

2. Transformation of Monocotyledons

Transformation of most monocotyledon species has now also become routine. Specific techniques include direct gene transfer into protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology 11: 1553-1558 (1993)) and Weeks et al. (Plant Physiol. 102: 1077-1084 (1993)) using particle bombardment of immature embryos and immature embryo-derived callus. A specific technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSG35) is precipitated onto micrometer size gold particles using standard procedures. Each plate of embryos is shot with the DuPont Biolistics® helium device using a burst pressure of ˜1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference. For this example, rice (Oryza sativa) is used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in quantity or substituted. Embryogenic responses are initiated and/or cultures are established from mature embryos by culturing on MS-CIM medium (MS basal salts, 4.3 g/liter; B5 vitamins (200×), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for ˜2 days at 28° C. Agrobacterium is re-suspended in liquid MS-CIM medium. The Agrobacterium culture is diluted to an OD600 of 0.2-0.3 and acetosyringone is added to a final concentration of 200 uM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (T0 generation) grown to maturity, and the T1 seed is harvested.

3. Transformation of Plastids

Seeds of Nicotiana tabacum c.v. ‘Xanthi nc’ are germinated seven per plate in a 1″ circular array on T agar medium and bombarded 12-14 days after sowing with 1 μm tungsten particles (M10, Biorad, Hercules, Calif.) coated with DNA from plasmids pPH143 and pPH145 essentially as described (Svab, Z. and Maliga, P. (1993) PNAS 90, 913-917). Bombarded seedlings are incubated on T medium for two days after which leaves are excised and placed abaxial side up in bright light (350-500 μmol photons/m2/s) on plates of RMOP medium (Svab, Z., Hajdukiewicz, P. and Maliga, P. (1990) PNAS 87, 8526-8530) containing 500 μg/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346-349) is separated on 1% Tris-borate (TBE) agarose gels, transferred to nylon membranes (Amersham) and probed with 32P-labeled random primed DNA sequences corresponding to a 0.7 kb BamHI/HindIII DNA fragment from pC8 containing a portion of the rps7/12 plastid targeting sequence. Homoplasmic shoots are rooted aseptically on spectinomycin-containing MS/IBA medium (McBride, K. E. et al. (1994) PNAS 91, 7301-7305) and transferred to the greenhouse.

### V. Breeding and Seed Production

### A. Breeding

The plants obtained via transformation with a nucleic acid sequence of the present invention can be any of a wide variety of plant species, including those of monocots and dicots; however, the plants used in the method of the invention are specifically selected from the list of agronomically important target crops set forth supra. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

The genetic properties engineered into the transgenic seeds and plants described above are passed on by sexual reproduction or vegetative growth and can thus be maintained and propagated in progeny plants. Generally said maintenance and propagation make use of known agricultural methods developed to fit specific purposes such as tilling, sowing or harvesting. Specialized processes such as hydroponics or greenhouse technologies can also be applied. As the growing crop is vulnerable to attack and damages caused by insects or infections as well as to competition by weed plants, measures are undertaken to control weeds, plant diseases, insects, nematodes, and other adverse conditions to improve yield. These include mechanical measures such a tillage of the soil or removal of weeds and infected plants, as well as the application of agrochemicals such as herbicides, fungicides, gametocides, nematicides, growth regulants, ripening agents and insecticides.

Use of the advantageous genetic properties of the transgenic plants and seeds according to the invention can further be made in plant breeding, which aims at the development of plants with improved properties such as tolerance of pests, herbicides, or stress, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants. Depending on the desired properties, different breeding measures are taken. The relevant techniques are well known in the art and include but are not limited to hybridization, inbreeding, backcross breeding, multiline breeding, variety blend, interspecific hybridization, aneuploid techniques, etc. Hybridization techniques also include the sterilization of plants to yield male or female sterile plants by mechanical, chemical, or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines, that for example, increase the effectiveness of conventional methods such as herbicide or pesticide treatment or allow one to dispense with said methods due to their modified genetic properties. Alternatively new crops with improved stress tolerance can be obtained, which, due to their optimized genetic “equipment”, yield harvested product of better quality than products that were not able to tolerate comparable adverse developmental conditions.

### B. Seed Production

In seed production, germination quality and uniformity of seeds are essential product characteristics. As it is difficult to keep a crop free from other crop and weed seeds, to control seedborne diseases, and to produce seed with good germination, fairly extensive and well-defined seed production practices have been developed by seed producers, who are experienced in the art of growing, conditioning and marketing of pure seed. Thus, it is common practice for the farmer to buy certified seed meeting specific quality standards instead of using seed harvested from his own crop. Propagation material to be used as seeds is customarily treated with a protectant coating comprising herbicides, insecticides, fungicides, bactericides, nematicides, molluscicides, or mixtures thereof. Customarily used protectant coatings comprise compounds such as captan, carboxin, thiram (TMTD®), methalaxyl (Aprone), and pirimiphos—methyl (Actellic®). If desired, these compounds are formulated together with further carriers, surfactants or application-promoting adjuvants customarily employed in the art of formulation to provide protection against damage caused by bacterial, fungal or animal pests. The protectant coatings may be applied by impregnating propagation material with a liquid formulation or by coating with a combined wet or dry formulation. Other methods of application are also possible such as treatment directed at the buds or the fruit.

### VI. Alteration of Expression of Nucleic Acid Molecules

The alteration in expression of the nucleic acid molecules of the present invention is achieved in one of the following ways:

A. “Sense” Suppression

Alteration of the expression of a nucleotide sequence of the present invention, specifically reduction of its expression, is obtained by “sense” suppression (referenced in e.g. Jorgensen et al. (1996) Plant Mol. Biol. 31, 957-973). In this case, the entirety or a portion of a nucleotide sequence of the present invention is comprised in a DNA molecule. The DNA molecule is specifically operatively linked to a promoter functional in a cell comprising the target gene, specifically a plant cell, and introduced into the cell, in which the nucleotide sequence is expressible. The nucleotide sequence is inserted in the DNA molecule in the “sense orientation”, meaning that the coding strand of the nucleotide sequence can be transcribed. In a specific embodiment, the nucleotide sequence is fully translatable and all the genetic information comprised in the nucleotide sequence, or portion thereof, is translated into a polypeptide. In another specific embodiment, the nucleotide sequence is partially translatable and a short peptide is translated. In a specific embodiment, this is achieved by inserting at least one premature stop codon in the nucleotide sequence, which bring translation to a halt. In another more specific embodiment, the nucleotide sequence is transcribed but no translation product is being made. This is usually achieved by removing the start codon, e.g. the “ATG”, of the polypeptide encoded by the nucleotide sequence. In a further specific embodiment, the DNA molecule comprising the nucleotide sequence, or a portion thereof, is stably integrated in the genome of the plant cell. In another specific embodiment, the DNA molecule comprising the nucleotide sequence, or a portion thereof, is comprised in an extrachromosomally replicating molecule.

In transgenic plants containing one of the DNA molecules described immediately above, the expression of the nucleotide sequence corresponding to the nucleotide sequence comprised in the DNA molecule is specifically reduced. Specifically, the nucleotide sequence in the DNA molecule is at least 70% identical to the nucleotide sequence the expression of which is reduced, more specifically it is at least 80% identical, yet more specifically at least 90% identical, yet more specifically at least 95% identical, yet more specifically at least 99% identical.

B. “Anti-Sense” Suppression

In another specific embodiment, the alteration of the expression of a nucleotide sequence of the present invention, specifically the reduction of its expression is obtained by “anti-sense” suppression. The entirety or a portion of a nucleotide sequence of the present invention is comprised in a DNA molecule. The DNA molecule is specifically operatively linked to a promoter functional in a plant cell, and introduced in a plant cell, in which the nucleotide sequence is expressible. The nucleotide sequence is inserted in the DNA molecule in the “anti-sense orientation”, meaning that the reverse complement (also called sometimes non-coding strand) of the nucleotide sequence can be transcribed. In a specific embodiment, the DNA molecule comprising the nucleotide sequence, or a portion thereof, is stably integrated in the genome of the plant cell. In another specific embodiment the DNA molecule comprising the nucleotide sequence, or a portion thereof, is comprised in an extrachromosomally replicating molecule. Several publications describing this approach are cited for further illustration (Green, P. J. et al., Ann. Rev. Biochem. 55:569-597 (1986); van der Krol, A. R. et al, Antisense Nuc. Acids & Proteins, pp. 125-141 (1991); Abel, P. P. et al., PNASroc. Natl. Acad. Sci. USA 86:6949-6952 (1989); Ecker, J. R. et al., Proc. Natl. Acad. Sci. USANAS 83:5372-5376 (August 1986)).

In transgenic plants containing one of the DNA molecules described immediately above, the expression of the nucleotide sequence corresponding to the nucleotide sequence comprised in the DNA molecule is specifically reduced. Specifically, the nucleotide sequence in the DNA molecule is at least 70% identical to the nucleotide sequence the expression of which is reduced, more specifically it is at least 80% identical, yet more specifically at least 90% identical, yet more specifically at least 95% identical, yet more specifically at least 99% identical.

C. Homologous Recombination

In another specific embodiment, at least one genomic copy corresponding to a nucleotide sequence of the present invention is modified in the genome of the plant by homologous recombination as further illustrated in Paszkowski et al., EMBO Journal 7:4021-26 (1988). This technique uses the property of homologous sequences to recognize each other and to exchange nucleotide sequences between each by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one embodiment, the regulatory elements of the nucleotide sequence of the present invention are modified. Such regulatory elements are easily obtainable by screening a genomic library using the nucleotide sequence of the present invention, or a portion thereof, as a probe. The existing regulatory elements are replaced by different regulatory elements, thus altering expression of the nucleotide sequence, or they are mutated or deleted, thus abolishing the expression of the nucleotide sequence. In another embodiment, the nucleotide sequence is modified by deletion of a part of the nucleotide sequence or the entire nucleotide sequence, or by mutation. Expression of a mutated polypeptide in a plant cell is also contemplated in the present invention. More recent refinements of this technique to disrupt endogenous plant genes have been described (Kempin et al., Nature 389:802-803 (1997) and Miao and Lam, Plant J., 7:359-365 (1995).

In another specific embodiment, a mutation in the chromosomal copy of a nucleotide sequence is introduced by transforming a cell with a chimeric oligonucleotide composed of a contiguous stretch of RNA and DNA residues in a duplex conformation with double hairpin caps on the ends. An additional feature of the oligonucleotide is for example the presence of 2′-O-methylation at the RNA residues. The RNA/DNA sequence is designed to align with the sequence of a chromosomal copy of a nucleotide sequence of the present invention and to contain the desired nucleotide change. For example, this technique is further illustrated in U.S. Pat. No. 5,501,967 and Zhu et al. (1999) Proc. Natl. Acad. Sci. USA 96: 8768-8773.

D. Ribozymes

In a further embodiment, the RNA coding for a polypeptide of the present invention is cleaved by a catalytic RNA, or ribozyme, specific for such RNA. The ribozyme is expressed in transgenic plants and results in reduced amounts of RNA coding for the polypeptide of the present invention in plant cells, thus leading to reduced amounts of polypeptide accumulated in the cells. This method is further illustrated in U.S. Pat. No. 4,987,071.

E. Dominant-Negative Mutants

In another specific embodiment, the activity of the polypeptide encoded by the nucleotide sequences of this invention is changed. This is achieved by expression of dominant negative mutants of the proteins in transgenic plants, leading to the loss of activity of the endogenous protein.

F. Aptamers

In a further embodiment, the activity of polypeptide of the present invention is inhibited by expressing in transgenic plants nucleic acid ligands, so-called aptamers, which specifically bind to the protein. Aptamers are preferentially obtained by the SELEX (Systematic Evolution of Ligands by EXponential Enrichment) method. In the SELEX method, a candidate mixture of single stranded nucleic acids having regions of randomized sequence is contacted with the protein and those nucleic acids having an increased affinity to the target are partitioned from the remainder of the candidate mixture. The partitioned nucleic acids are amplified to yield a ligand enriched mixture. After several iterations a nucleic acid with optimal affinity to the polypeptide is obtained and is used for expression in transgenic plants. This method is further illustrated in U.S. Pat. No. 5,270,163.

G. Zinc Finger Proteins

A zinc finger protein that binds a nucleotide sequence of the present invention or to its regulatory region is also used to alter expression of the nucleotide sequence. Specifically, transcription of the nucleotide sequence is reduced or increased. Zinc finger proteins are for example described in Beerli et al. (1998) PNAS 95:14628-14633., or in WO 95/19431, WO 98/54311, or WO 96/06166, all incorporated herein by reference in their entirety.

H. dsRNA

Alteration of the expression of a nucleotide sequence of the present invention is also obtained by dsRNA interference as described for example in WO 99/32619, WO 99/53050 or WO 99/61631, all incorporated herein by reference in their entirety. In another specific embodiment, the alteration of the expression of a nucleotide sequence of the present invention, specifically the reduction of its expression, is obtained by double-stranded RNA (dsRNA) interference. The entirety or, specifically a portion of a nucleotide sequence of the present invention is comprised in a DNA molecule. The size of the DNA molecule is specifically from 100 to 1000 nucleotides or more; the optimal size to be determined empirically. Two copies of the identical DNA molecule are linked, separated by a spacer DNA molecule, such that the first and second copies are in opposite orientations. In the specific embodiment, the first copy of the DNA molecule is in the reverse complement (also known as the non-coding strand) and the second copy is the coding strand; in the most specific embodiment, the first copy is the coding strand, and the second copy is the reverse complement. The size of the spacer DNA molecule is specifically 200 to 10,000 nucleotides, more specifically 400 to 5000 nucleotides and most specifically 600 to 1500 nucleotides in length. The spacer is specifically a random piece of DNA, more specifically a random piece of DNA without homology to the target organism for dsRNA interference, and most specifically a functional intron which is effectively spliced by the target organism. The two copies of the DNA molecule separated by the spacer are operatively linked to a promoter functional in a plant cell, and introduced in a plant cell, in which the nucleotide sequence is expressible. In a specific embodiment, the DNA molecule comprising the nucleotide sequence, or a portion thereof, is stably integrated in the genome of the plant cell. In another specific embodiment the DNA molecule comprising the nucleotide sequence, or a portion thereof, is comprised in an extrachromosomally replicating molecule. Several publications describing this approach are cited for further illustration (Waterhouse et al. (1998) PNAS 95:13959-13964; Chuang and Meyerowitz (2000) PNAS 97:4985-4990; Smith et al. (2000) Nature 407:319-320). Alteration of the expression of a nucleotide sequence by dsRNA interference is also described in, for example WO 99/32619, WO 99/53050 or WO 99/61631, all incorporated herein by reference in their entirety.

In transgenic plants containing one of the DNA molecules described immediately above, the expression of the nucleotide sequence corresponding to the nucleotide sequence comprised in the DNA molecule is specifically reduced. Specifically, the nucleotide sequence in the DNA molecule is at least 70% identical to the nucleotide sequence the expression of which is reduced, more specifically it is at least 80% identical, yet more specifically at least 90% identical, yet more specifically at least 95% identical, yet more specifically at least 99% identical.

I. Insertion of a DNA Molecule (Insertional Mutagenesis)

In another specific embodiment, a DNA molecule is inserted into a chromosomal copy of a nucleotide sequence of the present invention, or into a regulatory region thereof. Specifically, such DNA molecule comprises a transposable element capable of transposition in a plant cell, such as e.g. Ac/Ds, Em/Spm, mutator. Alternatively, the DNA molecule comprises a T-DNA border of an Agrobacterium T-DNA. The DNA molecule may also comprise a recombinase or integrase recognition site which can be used to remove part of the DNA molecule from the chromosome of the plant cell. Methods of insertional mutagenesis using T-DNA, transposons, oligonucleotides or other methods known to those skilled in the art are also encompassed. Methods of using T-DNA and transposon for insertional mutagenesis are described in Winkler et al. (1989) Methods Mol. Biol. 82:129-136 and Martienssen (1998) PNAS 95:2021-2026, incorporated herein by reference in their entireties.

J. Deletion Mutagenesis

In yet another embodiment, a mutation of a nucleic acid molecule of the present invention is created in the genomic copy of the sequence in the cell or plant by deletion of a portion of the nucleotide sequence or regulator sequence. Methods of deletion mutagenesis are known to those skilled in the art. See, for example, Miao et al, (1995) Plant J. 7:359.

In yet another embodiment, this deletion is created at random in a large population of plants by chemical mutagenesis or irradiation and a plant with a deletion in a gene of the present invention is isolated by forward or reverse genetics. Irradiation with fast neutrons or gamma rays is known to cause deletion mutations in plants (Silverstone et al, (1998) Plant Cell, 10:155-169; Bruggemann et al., (1996) Plant J., 10:755-760; Redei and Koncz in Methods in Arabidopsis Research, World Scientific Press (1992), pp. 16-82). Deletion mutations in a gene of the present invention can be recovered in a reverse genetics strategy using PCR with pooled sets of genomic DNAs as has been shown in C. elegans (Liu et al., (1999), Genome Research, 9:859-867.). A forward genetics strategy would involve mutagenesis of a line displaying PTGS followed by screening the M2 progeny for the absence of PTGS. Among these mutants would be expected to be some that disrupt a gene of the present invention. This could be assessed by Southern blot or PCR for a gene of the present invention with genomic DNA from these mutants.

K. Overexpression in a Plant Cell

In yet another specific embodiment, a nucleotide sequence of the present invention encoding a polypeptide is over-expressed. Examples of nucleic acid molecules and expression cassettes for over-expression of a nucleic acid molecule of the present invention are described above. Methods known to those skilled in the art of over-expression of nucleic acid molecules are also encompassed by the present invention.

In a specific embodiment, the expression of the nucleotide sequence of the present invention is altered in every cell of a plant. This is for example obtained though homologous recombination or by insertion in the chromosome. This is also for example obtained by expressing a sense or antisense RNA, zinc finger protein or ribozyme under the control of a promoter capable of expressing the sense or antisense RNA, zinc finger protein or ribozyme in every cell of a plant. Constitutive expression, inducible, tissue-specific or developmentally-regulated expression are also within the scope of the present invention and result in a constitutive, inducible, tissue-specific or developmentally-regulated alteration of the expression of a nucleotide sequence of the present invention in the plant cell. Constructs for expression of the sense or antisense RNA, zinc finger protein or ribozyme, or for over-expression of a nucleotide sequence of the present invention, are prepared and transformed into a plant cell according to the teachings of the present invention, e.g. as described infra.

### VII. Polypeptides

The present invention further relates to isolated polypeptides comprising the amino acid sequence of SEQ ID NOs: 2, 4, 6, 8, 10. In particular, isolated polypeptides comprising the amino acid sequence of SEQ ID NOs: 2, 4, 6, 8, 10, and variants having conservative amino acid modifications. One skilled in the art will recognize that individual substitutions, deletions or additions to a nucleic acid, peptide, polypeptide or protein sequence which alters, adds or deletes a single amino acid or a small percent of amino acids in the encoded sequence is a “conservative modification” where the modification results in the substitution of an amino acid with a chemically similar amino acid. Conservative modified variants provide similar biological activity as the unmodified polypeptide. Conservative substitution tables listing functionally similar amino acids are known in the art. See Crighton (1984) Proteins, W.H. Freeman and Company.

In a specific embodiment, a polypeptide having substantial similarity to a polypeptide sequence of SEQ ID NO:2, including the polypeptide sequences of SEQ ID NO:4, SEQ ID NO:6, SEQ ID NO:8 or SEQ ID NO:10, or exon or domain thereof, is an allelic variant of the polypeptide sequence listed in SEQ ID NO:2. In another specific embodiment, a polypeptide having substantial similarity to a polypeptide sequence listed in SEQ ID NO:2, or exon or domain thereof, is a naturally occurring variant of the polypeptide sequence listed SEQ ID NO:2. In another specific embodiment, a polypeptide having substantial similarity to a polypeptide sequence listed SEQ ID NO:2, or exon or domain thereof, is a polymorphic variant of the polypeptide sequence listed in SEQ ID NO:2.

In an alternate specific embodiment, the sequence having substantial similarity contains a deletion or insertion of at least one amino acid. In a more specific embodiment, the deletion or insertion is of less than about ten amino acids. In a most specific embodiment, the deletion or insertion is of less than about three amino acids.

In a specific embodiment, the sequence having substantial similarity encodes a substitution in at least one amino acid.

In another specific embodiment, the polypeptide having substantial similarity is an allelic variant of a polypeptide sequence listed in SEQ ID NO:2, or a fragment, domain, repeat or chimeras thereof. In another specific embodiment, the isolated nucleic acid includes a plurality of regions from the polypeptide sequence encoded by a nucleotide sequence identical to or having substantial similarity to a nucleotide sequence listed in SEQ ID NO:1, or fragment or domain thereof, or a sequence complementary thereto.

In another specific embodiment, the polypeptide is a polypeptide sequence listed in SEQ ID NO:2. In another specific embodiment, the polypeptide is a functional fragment or domain. In yet another specific embodiment, the polypeptide is a chimera, where the chimera may include functional protein domains, including domains, repeats, post-translational modification sites, or other features. In a more specific embodiment, the polypeptide is a plant polypeptide. In a more specific embodiment, the plant is a dicot. In a more specific embodiment, the plant is a gymnosperm. In a more specific embodiment, the plant is a monocot. In a more specific embodiment, the monocot is a cereal. In a more specific embodiment, the cereal may be, for example, maize, wheat, barley, oats, rye, millet, sorghum, triticale, secale, einkorn, spelt, emmer, teff, milo, flax, gramma grass, Tripsacum, and teosinte. In another specific embodiment, the cereal is rice.

In a specific embodiment, the polypeptide is expressed in a specific location or tissue of a plant. In a more specific embodiment, the location or tissue is for example, but not limited to, epidermis, vascular tissue, meristem, cambium, cortex or pith. In a most specific embodiment, the location or tissue is leaf or sheath, root, flower, and developing ovule or seed. In a more specific embodiment, the location or tissue may be, for example, epidermis, root, vascular tissue, meristem, cambium, cortex, pith, leaf, and flower. In a more specific embodiment, the location or tissue is a seed.

In a specific embodiment, the polypeptide sequence encoded by a nucleotide sequence having substantial similarity to a nucleotide sequence listed in SEQ ID NO:1 including the nucleotide sequences of SEQ ID NO:3, SEQ ID NO:5, SEQ ID NO:7, or SEQ ID NO:9 or a fragment or domain thereof or a sequence complementary thereto, is a mutant variant of the nucleotide sequence listed in SEQ ID NO:1 and includes a substitution, deletion or insertion of at least one nucleotide. In a more specific embodiment, the deletion or insertion is of less than about thirty nucleotides. In a most specific embodiment, the deletion or insertion is of less than about five nucleotides.

In a specific embodiment, the polypeptide sequence encoded by a nucleotide sequence having substantial similarity to a nucleotide sequence listed in SEQ ID NOs:1, 3, 5, 7, 9, or fragment or domain thereof or a sequence complementary thereto, includes a substitution of at least one codon. In a more specific embodiment, the substitution is conservative.

In a specific embodiment, the polypeptide sequences having substantial similarity to the polypeptide sequence listed in SEQ ID NO:2, or a fragment, domain, repeat or chimeras thereof includes a substitution, deletion or insertion of at least one amino acid.

The polypeptides of the invention, fragments thereof or variants thereof can comprise any number of contiguous amino acid residues from a polypeptide of the invention, wherein the number of residues is selected from the group of integers consisting of from 10 to the number of residues in a full-length polypeptide of the invention. Specifically, the portion or fragment of the polypeptide is a functional protein. The present invention includes active polypeptides having specific activity of at least 20%, 30%, or 40%, and specifically at least 505, 60%, or 70%, and most specifically at least 805, 90% or 95% that of the native (non-synthetic) endogenous polypeptide. Further, the substrate specificity (kcat/Km) is optionally substantially similar to the native (non-synthetic), endogenous polypeptide. Typically the Km will be at least 30%, 40%, or 50% of the native, endogenous polypeptide; and more specifically at least 605, 70%, 80%, or 90%. Methods of assaying and quantifying measures of activity and substrate specificity are well known to those of skill in the art.

The isolated polypeptides of the present invention will elicit production of an antibody specifically reactive to a polypeptide of the present invention when presented as an immunogen. Therefore, the polypeptides of the present invention can be employed as immunogens for constructing antibodies immunoreactive to a protein of the present invention for such purposes, but not limited to, immunoassays or protein purification techniques. Immunoassays for determining binding are well known to those of skill in the art such as, but not limited to, ELISAs or competitive immunoassays.

Embodiments of the present invention also relate to chimeric polypeptides encoded by the isolated nucleic acid molecules of the present disclosure including a chimeric polypeptide containing a polypeptide sequence encoded by an isolated nucleic acid containing a nucleotide sequence including:

(a) a nucleotide sequence listed in SEQ ID NOs:1, 3, 5, 7, 9, or an exon or domain thereof;

(b) a nucleotide sequence having substantial similarity to (a);

(c) a nucleotide sequence capable of hybridizing to (a);

(d) a nucleotide sequence complementary to (a), (b) or (c); and

(e) a nucleotide sequence which is the reverse complement of (a), (b) or (c); or

(f) a functional fragment thereof.

A polypeptide containing a polypeptide sequence encoded by an isolated nucleic acid containing a nucleotide sequence, its complement, or its reverse complement, encoding a polypeptide including a polypeptide sequence including:

(a) a polypeptide sequence listed in SEQ ID NO:2, or a domain, repeat or chimeras thereof;

(b) a polypeptide sequence having substantial similarity to (a), including the polypeptide sequences listed in SEQ ID NO:4, SEQ ID NO:6, SEQ ID NO:8, and SEQ ID NO:10;

(c) a polypeptide sequence encoded by a nucleotide sequence identical to or having substantial similarity to a nucleotide sequence listed in SEQ ID NOs:1, 3, 5, 7, 9, or an exon or domain thereof, or a sequence complementary thereto;

(d) a polypeptide sequence encoded by a nucleotide sequence capable of hybridizing under medium stringency conditions to a nucleotide sequence listed in SEQ ID NOs:1, 3, 5, 7, 9, or to a sequence complementary thereto; and a functional fragment of (a), (b), (c) or (d); or

(e) a functional fragment thereof.

The isolated nucleic acid molecules of the present invention are useful for expressing a polypeptide of the present invention in a recombinantly engineered cell such as a bacteria, yeast, insect, mammalian or plant cell. The cells produce the polypeptide in a non-natural condition (e.g. in quantity, composition, location and/or time) because they have been genetically altered to do so. Those skilled in the art are knowledgeable in the numerous expression systems available for expression of nucleic acids encoding a protein of the present invention, and will not be described in detail below.

Briefly, the expression of isolated nucleic acids encoding a polypeptide of the invention will typically be achieved, for example, by operably linking the nucleic acid or cDNA to a promoter (constitutive or regulatable) followed by incorporation into an expression vector. The vectors are suitable for replication and/or integration in either prokaryotes or eukaryotes. Commonly used expression vectors comprise transcription and translation terminators, initiation sequences and promoters for regulation of the expression of the nucleic acid molecule encoding the polypeptide. To obtain high levels of expression of the cloned nucleic acid molecule, it is desirable to use expression vectors comprising a strong promoter to direct transcription, a ribosome binding site for translation initiation, and a transcription/translation terminator. One skilled in the art will recognize that modifications may be made to the polypeptide of the present invention without diminishing its biological activity. Some modifications may be made to facilitate the cloning, expression or incorporation of the polypeptide of the invention into a fusion protein. Such modification are well known in the art and include, but are not limited to, a methionine added at the amino terminus to provide an initiation site, or additional amino acids (e.g. poly histidine) placed on either terminus to create conveniently located purification sequences. Restriction sites or termination codons can also be introduced into the vector.

In a specific embodiment, the expression vector includes one or more elements such as, for example, but not limited to, a promoter-enhancer sequence, a selection marker sequence, an origin of replication, an epitope-tag encoding sequence, or an affinity purification-tag encoding sequence. In a more specific embodiment, the promoter-enhancer sequence may be, for example, the CaMV 35S promoter, the CaMV 19S promoter, the tobacco PR-1a promoter, the ubiquitin promoter, and the phaseolin promoter. In another embodiment, the promoter is operable in plants, and more specifically, a constitutive or inducible promoter. In another specific embodiment, the selection marker sequence encodes an antibiotic resistance gene. In another specific embodiment, the epitope-tag sequence encodes V5, the peptide Phe-His-His-Thr-Thr, hemagglutinin, or glutathione-S-transferase. In another specific embodiment the affinity purification-tag sequence encodes a polyamino acid sequence or a polypeptide. In a more specific embodiment, the polyamino acid sequence is polyhistidine. In a more specific embodiment, the polypeptide is chitin binding domain or glutathione-S-transferase. In a more specific embodiment, the affinity purification-tag sequence comprises an intein encoding sequence.

Prokaryotic cells may be used a host cells, for example, but not limited to, Escherichia coli, and other microbial strains known to those in the art. Methods for expressing proteins in prokaryotic cells are well known to those in the art and can be found in many laboratory manuals such as Molecular Cloning: A Laboratory Manual, by J. Sambrook et al. (1989, Cold Spring Harbor Laboratory Press). A variety of promoters, ribosome binding sites, and operators to control expression are available to those skilled in the art, as are selectable markers such as antibiotic resistance genes. The type of vector chosen is to allow for optimal growth and expression in the selected cell type.

A variety of eukaryotic expression systems are available such as, but not limited to, yeast, insect cell lines, plant cells and mammalian cells. Expression and synthesis of heterologous proteins in yeast is well known (see Sherman et al., Methods in Yeast Genetics, Cold Spring Harbor Laboratory Press, 1982). Commonly used yeast strains widely used for production of eukaryotic proteins are Saccharomyces cerevisiae and Pichia pastoris, and vectors, strains and protocols for expression are available from commercial suppliers (e.g., Invitrogen).

Mammalian cell systems may be transfected with expression vectors for production of proteins. Many suitable host cell lines are available to those in the art, such as, but not limited to the HEK293, BHK21 and CHO cells lines. Expression vectors for these cells can include expression control sequences such as an origin of replication, a promoter, (e.g., the CMV promoter, a HSV tk promoter or phosphoglycerate kinase (pgk) promoter), an enhancer, and protein processing sites such as ribosome binding sites, RNA splice sites, polyadenylation sites, and transcription terminator sequences. Other animal cell lines useful for the production of proteins are available commercially or from depositories such as the American Type Culture Collection.

Expression vectors for expressing proteins in insect cells are usually derived from the SF9 baculovirus or other viruses known in the art. A number of suitable insect cell lines are available including but not limited to, mosquito larvae, silkworm, armyworm, moth and Drosophila cell lines.

Methods of transfecting animal and lower eukaryotic cells are known. Numerous methods are used to make eukaryotic cells competent to introduce DNA such as but not limited to: calcium phosphate precipitation, fusion of the recipient cell with bacterial protoplasts containing the DNA, treatment of the recipient cells with liposomes containing the DNA, DEAE dextrin, electroporation, biolistics, and microinjection of the DNA directly into the cells. Transfected cells are cultured using means well known in the art (see, Kuchler, R. J., Biochemical Methods in Cell Culture and Virology, Dowden, Hutchinson and Ross, Inc. 1997).

Once a polypeptide of the present invention is expressed it may be isolated and purified from the cells using methods known to those skilled in the art. The purification process may be monitored using Western blot techniques or radioimmunoassay or other standard immunoassay techniques. Protein purification techniques are commonly known and used by those in the art (see R. Scopes, Protein Purification: Principles and Practice, Springer-Verlag, New York 1982: Deutscher, Guide to Protein Purification, Academic Press (1990). Embodiments of the present invention provide a method of producing a recombinant protein in which the expression vector includes one or more elements including a promoter-enhancer sequence, a selection marker sequence, an origin of replication, an epitope-tag encoding sequence, and an affinity purification-tag encoding sequence. In one specific embodiment, the nucleic acid construct includes an epitope-tag encoding sequence and the isolating step includes use of an antibody specific for the epitope-tag. In another specific embodiment, the nucleic acid construct contains a polyamino acid encoding sequence and the isolating step includes use of a resin comprising a polyamino acid binding substance, specifically where the polyamino acid is polyhistidine and the polyamino binding resin is nickel-charged agarose resin. In yet another specific embodiment, the nucleic acid construct contains a polypeptide encoding sequence and the isolating step includes the use of a resin containing a polypeptide binding substance, specifically where the polypeptide is a chitin binding domain and the resin contains chitin-sepharose.

The polypeptides of the present invention can be synthesized using non-cellular synthetic methods known to those in the art. Techniques for solid phase synthesis are described by Barany and Mayfield, Solid-Phase Peptide Synthesis, pp. 3-284 in the Peptides: Analysis, Synthesis, Biology, Vol. 2, Special Methods in Peptide Synthesis, Part A; Merrifield, et al., J. Am. Chem. Soc. 85:2149-56 (1963) and Stewart et al., Solid Phase Peptide Synthesis, 2nd ed. Pierce Chem. Co., Rockford, Ill. (1984).

The present invention further provides a method for modifying (i.e. increasing or decreasing) the concentration or composition of the polypeptides of the invention in a plant or part thereof. Modification can be effected by increasing or decreasing the concentration and/or the composition (i.e. the ratio of the polypeptides of the present invention) in a plant. The method comprised introducing into a plant cell with an expression cassette comprising a nucleic acid molecule of the present invention, or a nucleic acid encoding the sequences of SEQ ID NOs:1, 3, 5, 7, 9 as described above to obtain a transformed plant cell or tissue, culturing the transformed plant cell or tissue. The nucleic acid molecule can be under the regulation of a constitutive or inducible promoter. The method can further comprise inducing or repressing expression of a nucleic acid molecule of a sequence in the plant for a time sufficient to modify the concentration and/or composition in the plant or plant part.

A plant or plant part having modified expression of a nucleic acid molecule of the invention can be analyzed and selected using methods known to those skilled in the art such as, but not limited to, Southern blot, DNA sequencing, or PCR analysis using primers specific to the nucleic acid molecule and detecting amplicons produced therefrom.

In general, concentration or composition in increased or decreased by at least 5%, 10%, 20%, 30%, 40%, 50%, 60%, 70%, 80% or 90% relative to a native control plant, plant part or cell lacking the expression cassette.

The adaptability to nitrogen limitation is an essential trait for plants and is positively correlated with crop yield. Numerous biotic and abiotic factors that consume nitrogen in the soil frequently create a nitrogen limitation growth condition. To cope with this, plants have evolved a suite of nitrogen limitation adaptive responses. However, knowledge is limited on the physiological and biochemical changes involved in these adaptive responses, and nothing has previously been known about the molecular mechanism governing plant adaptability to nitrogen limitation. The RING domain protein disclosed here is involved in mediating the adaptive response of plants to nitrogen limitation. Increased expression of this gene can produce plants with increased yield, particularly as the manipulation of nitrogen limitation adaptability can lead to enhanced nitrogen utilization and alter source-sink relationships in seeds, tubes, roots and other storage organs.

The invention will be further described by reference to the following detailed examples. These examples are provided for purposes of illustration only, and are not intended to be limiting unless otherwise specified.

### EXAMPLES

Standard recombinant DNA and molecular cloning techniques used here are well known in the art and are described by J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

### Example 1

### Plant Growth Conditions and Isolation of the Lines Mutant

A collection of Arabidopsis homozygous T-DNA insertion mutant lines (in Columbia background) were identified from ABRC seed stocks. In a growth room with controlled environmental conditions (23° C. day/18° C. night, white fluorescent illumination of 150 μmol/m²·s, 16 hr light/8 hr dark, and 75% relative humidity), these T-DNA lines were grown in the nutrient-free soil LB2 (SunGro Horticulture Canada Ltd. BC. Canada) supplied with 3 or 10 mM potassium nitrate in the nutrient solution (10 mM KH₂PO₄ pH5.6, 2 mM MgSO₄, 1 mM CaCl₂, 0.1 mM Fe-EDTA, 50 μM H₃BO₄, 12 μM MnSO₄, 1 μM ZnCl₂, 1 μM CuSO₄, 0.2 μM Na₂MoO₄) once a week for four weeks. Based on the low nitrate induced early senescence phenotype, the lines mutant was isolated from these T-DNA lines.

### Biochemical Analysis

The 5^{th}-8^{th} rosette leaves from lines and Col plants grown in LB2 soil with 3 mM nitrate for different days were harvested, frozen in liquid nitrogen, and stored at −80° C. for the following biochemical analysis. Nitrate was extracted from the frozen leaves and assayed according to Clothern et al., (1975). Total amino acids were extracted successively with 80%, 50%, 0% ethonal in HEPES-KOH buffer (pH 7.4), and the pooled supernatants were used for total amino acids assay as described by Rosen (1957). To extract soluble proteins, the frozen leaf power was suspended in 100 mM HEPES-KOH (pH 7.5)+0.1% Triton X-100 buffer and centrifuged at 14,000 rpm for 10 min. Total soluble protein contents in the supernatants were determined using the commercial protein assay kit (Bio-Rad, Hercules, Calif.). To determine nitrogen content, an aliquot of the frozen leaf powder was vacuum dried overnight, and total nitrogen in 1.5 mg dry powder were measured by Micro-Dumas combustion analysis method using the NA1500 C/H/N Analyzer (Carlo Erba Strumentazione, Milan, Italia). Soluble sugar was extracted as described by Geiger et al. (1998), and assayed for glucose, fructose and sucrose contents using a commercially available kit (Megazyme, Ireland). To analyze chlorophyll, the frozen leaf powder was suspended in 80% acetone, and centrifuged at 13,000 rpm. The extraction was repeated twice, and the chlorophyll content in the pooled supernatant was measured according to Amon (1949). Anthocyanin was extracted from the frozen leaf powder and determined as described in Noh and Spalding (1998).

### Expression Analysis by RT-PCR

Total RNA was extracted from various Arabidopsis plant tissues using TriZol reagent (Invitrogen). The first-strand cDNA was synthesized from total RNA samples with a kit (Fermentas) and used for PCR. The expression of Arabidopsis genes involved in nitrate metabolism (NR1, NR2, GS2, NRT1.1, NRT2.1), photosynthesis (RBCS and CAB1), anthocyanin synthesis (CHS), and senescence (SAG12), was detected by the semi-quantitative PT-PCR, and ubiquitin-10 expression was used as the internal control. The specific primers and PCR conditions for these genes are available when required.

### Map-Based Cloning of LINES Gene

One homozygous lines plant (in Col background) was crossed with a Landsberg erecta wild type plant. Among the segregating F2 progeny, which was grown in LB2 soil with 3 mM KNO₃, 518 plants showing lines mutant phenotype were selected for PCR-based mapping. First round mapping was performed according to Lukowitz et al. (2000). SSLP and CAPS markers for the following fine mapping were developed from Arabidopsis genome sequence database (www.arabidopsis.org).

### Generation of Transgenic Arabidopsis Plants

To determine whether At1g02860 is the LINES gene, the coding sequence of At1g02860 was amplified by RT-PCR using one pair of primers LINEScDNA-F 5′ ACA ACC GGT TTG AGG GCT GM TTT GTT TG 3′ (SEQ ID NO:11) and LINEScDNAR 5′ ACA GM TTC TAT ATC ATA TTC CAG TGA AGC T 3′(SEQ ID NO:12). The PCR product was cloned into the Age I and EcoR I sites in the binary vector pEGAD (Cutler et al., 2000), where At1g02860 cDNA expression will be driven by 35S promoter in plants. The construct was transformed into lines mutant plants as described by Clough and Bent (1998), and the transformants were screened by spreading the T1 seedlings with the herbicide BASTA (1:500 dilution, Aventis, Strasbourg, France). T2 seeds from three independent T1 transgenic lines were sown in LB2 soil with 3 mM nitrate for phenotype testing.

### Results

### The Lines Mutant Displayed Low Inorganic Nitrogen-Induced Early Senescence Phenotype

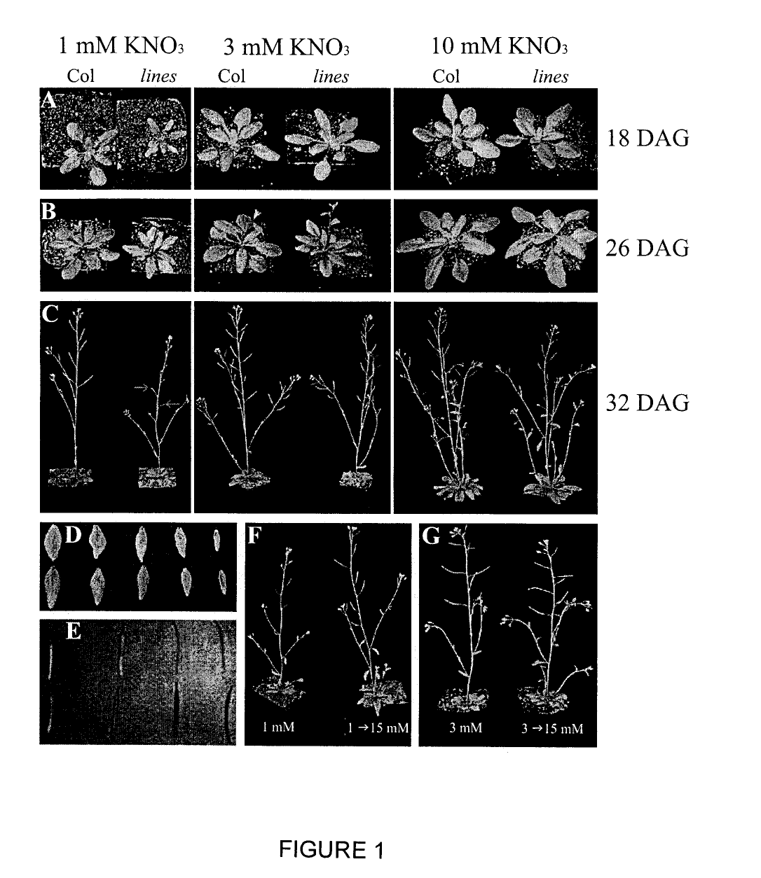

The major inorganic nitrogen compound available to crop plants under most soil conditions is nitrate (Crawford and Forde, 2002). To study how Arabidopsis plants respond to varying nitrate supply, a growth system was established where 10 mM nitrate provides sufficient nitrogen nutrient for Arabidopsis plants, while 3 mM nitrate limits the growth of Arabidopsis plants significantly (Bi et al., 2005). The following adaptive responses to insufficient nitrogen supply were observed. Arabidopsis plants supplied with 3 mM nitrate decreased their growth by 30% as compared to those supplied with 10 mM nitrate (Bi et al., 2005), and showed an increase in the redness of rosette leaves which indicates the accumulation of anthocyanin (FIG. 1A to C). Further, they initiated the senescence process in rosette leaves at least two weeks earlier than those grown with 10 mM nitrate (data not shown). To screen mutants with altered growth responses to the nitrogen limitation growth condition, a collection of homozygous T-DNA insertion lines were identified from the Arabidopsis Biological Resource Center (ABRC) seed stocks (Alonso et al., 2003), and grown in the nitrate application controlled system to evaluate their growth performance. One T-DNA insertion line failed to acclimatize to the nitrogen limitation growth condition, and started senescence much earlier and more rapidly than did wild type when supplied with 3 mM nitrate (FIG. 1). Accordingly, this T-DNA insertion line was called a low inorganic nitrogen-induced early senescence (lines) mutant. Shown in FIG. 1A to C, the lines mutant plants supplied with 10 mM nitrate had a similar growth and development pattern to wild type. When the nitrate concentration was reduced to 3 mM, the lines plants started senescence in the 5^{th} rosette leaf at 24 days after germination (DAG), and after this point senescence progressed rapidly with all rosette leaves showing senescence symptoms at 26 DAG, and the whole rosettes dying at 32 DAG. In contrast, wild type plants displayed no senescence symptoms in the 5^{th} rosette leaf until 32 DAG. In wild type plants, the senescence process proceeded slowly and gradually from the 5^{th} to the younger rosette leaves, and it took at least two weeks for all rosette leaves to show the senescence symptoms. The cauline leaves in the lines plants started senescence at 28 DAG, at least 10 days earlier than those in the wild type plants (FIG. 1D). Further, the developing lines siliques initiated senescence in their tips at 32 DAG, while the wild type siliques never showed senescence symptoms throughout their development, but accumulated abundant anthocyanin which was not observed in the lines siliques (FIG. 1E). With the reduction of the nitrate concentration to 1 mM, the occurrence of the senescence phenotype in the rosette leaves of lines plants was accelerated to 20 DAG, and severe senescence in the developing lines siliques resulted in their death around 30 DAG without producing viable seeds. Under the same growth condition with 1 mM nitrate, wild type plants did not start senescence in their rosette leaves until 26 DAG, and produced fecund siliques (FIG. 1C).

To further confirm that the early senescence phenotype in the lines mutant is dependent on insufficient nitrate supply, the inventors grew lines plants with 1 or 3 mM nitrate. When the senescence symptom was just initiated in the 5^{th} rosette leaves, these plants were supplied with 15 mM nitrate. Consequently, the senescence process in the senescing lines plants was stopped with the senesced rosette leaves renewing their growth and new rosette leaves being free of senescence symptoms. Further, new lateral shoot branches were produced, and no senescence symptoms were seen in the cauline leaves and the siliques (FIGS. 1F and G). Finally, siliques in these lines plants became fecund after they were supplied with 15 mM nitrate (FIG. 1F).

Besides nitrate, other inorganic nitrogen fertilizers include ammonium and ammonium nitrate. The inventors found that the early senescence phenotype of lines mutant was also induced by insufficient ammonium and ammonium nitrate supply. The lines plants grown on 20 mM ammonium or 10 mM ammonium nitrate had a similar growth and development pattern to that of wild type plants (data not shown). When grown on 5.0 mM ammonium or 2.5 mM ammonium nitrate, lines plants started the senescence process in the rosette leaves around 24 DAG, and subsequently senescence occurred rapidly in whole rosettes, cauline leaves, and siliques. Under the same nitrogen limitation conditions, wild type plants did not show obvious senescence symptom in the 5^{th} rosette leaf until 32 DAG (data not shown). Further, the insufficient ammonium and ammonium nitrate induced early senescence phenotype in lines mutant could also be rescued by supplying senescing lines plants with high amount of ammonium or ammonium nitrate (data not shown). Since the three inorganic nitrogen forms have the same effect on lines mutant, the inventors used nitrate as the nitrogen source in the following experiments.

### Map-Based Cloning of LINES Gene

To determine the inheritance of the low inorganic nitrogen induced early senescence phenotype in the lines mutant, the inventors backcrossed the lines mutant to wild type. The F1 plants showed the same phenotype as wild type plants when grown on 3 mM nitrate. In the F₂ generation, wild type and lines mutant phenotypes segregated at a ratio of 3:1 (data not shown), indicating that the lines mutant phenotype is recessive, and inherited as a single Mendelian trait. Southern blot analysis revealed that the genome of the lines mutant contained five T-DNA insertions, while none was genetically linked to the low nitrogen induced early senescence phenotype (data not shown), indicating the responsible gene in the lines mutant was not tagged by a T-DNA insertion. Subsequently, the lines mutant was successively backcrossed to wild type four times with no T-DNA insertion being left in the lines mutant.

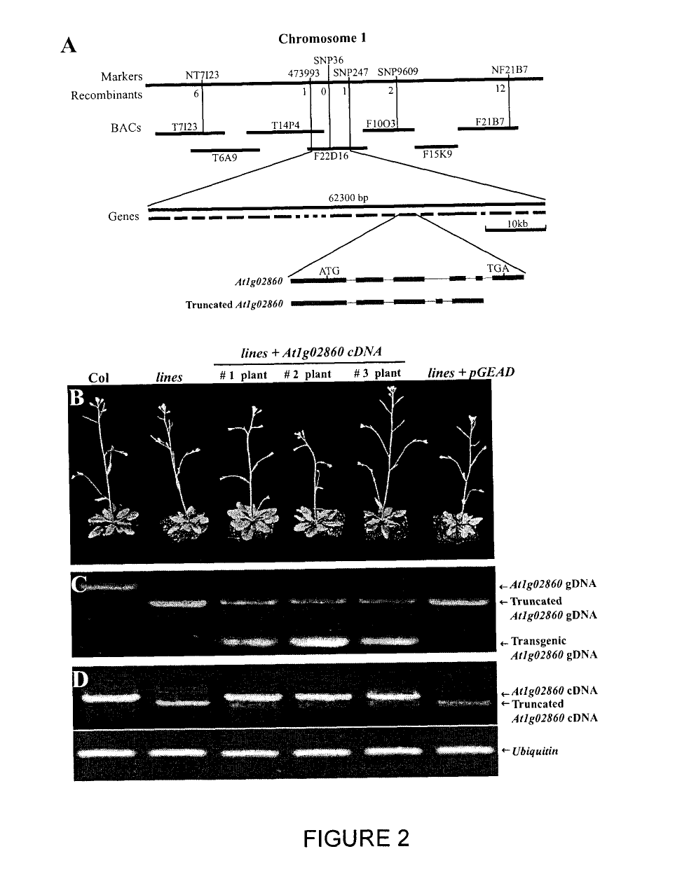

Because the LINES gene is not tagged by T-DNA insertion in the lines mutant, a map-based cloning approach was used to isolate LINES. A F2 mapping population was generated by crossing the lines mutant with Landsberg erecta wild type. Genomic DNA from 50 F2 lines mutant plants were bulked and used for the initial mapping with the 22 simple sequence length polymorphism (SSLP) markers from Lukowitz et al. (2000). A close linkage was detected between LINES and the SSLP market F21M12 on the top arm of chromosome 1 (data not shown). Further mapping with additional available SSLP markers (www.arabidopsis.org) located LINES in a region which is bordered by two SSLP markers NF21B7 and NT7123 and covered by seven BACs (FIG. 2A). Using the two SSLP markers, 18 recombinants were identified among 518 lines plants in the F2 mapping population. Fine mapping with these 18 recombinants and more SSLP and cleaved amplified polymorphic sequence (CAPS) markers narrowed down the position of the LINES locus to a genomic region between the SSLP marker 473993 and the CAPS marker SNP247 on the BAC clone F22D16. This region is approximately 62.3 kb and contains 21 annotated genes (FIG. 2A). Thirteen genes could be the candidate for LINES, and their coding regions and the corresponding genomic sequences were amplified from the lines mutant plants by RT-PCR and PCR, respectively. Comparing these PCR products with their counterparts from wild type revealed that only the gene At1g02860 was shortened in the genomic and coding sequences of the lines mutant (FIGS. 2C and D). Complete sequencing of the lines At1g02860 gene showed that the third intron and fourth exon of At1g02860 was deleted in the lines mutant (FIG. 2A), and the remaining exons 3 and 5 were fused in frame (FIG. 2A). This resulted in a truncated At1g02860 cDNA detected in the lines mutant (FIG. 2D).

To confirm that At1g02860 is indeed the LINES gene, the wild type At1g02860 cDNA driven by the 35S promoter was transformed into the lines mutant. PCR and RT-PCR analysis revealed three independent transformants contained both truncated At1g02860 genomic sequence (1.4 kb) and the transformed At1g02860 cDNA (1.0 kb) in their genomes, and correspondingly the truncated (0.9 kb) and wild type (1.0 kb) At1g02860 mRNA (FIGS. 2C and D). Similar to wild type plants, the three transformants did not show the early senescence phenotype when supplied with 3 mM nitrate (FIG. 2B). In contrast, the control lines plant transformed with the empty binary vector PGEAD (Culter et al., 2000) did not express the wild type At1g02860 cDNA (FIG. 2D), and displayed the low nitrogen induced early senescence phenotype (FIG. 2B). All the data unambiguously assign At1g02860 as the LINES gene, and the mutated At1g02860 gene is responsible for the low nitrogen induced early senescence phenotype in the lines mutant.

### LINES Encodes a RING-Type Ubiquitin E3 Ligase

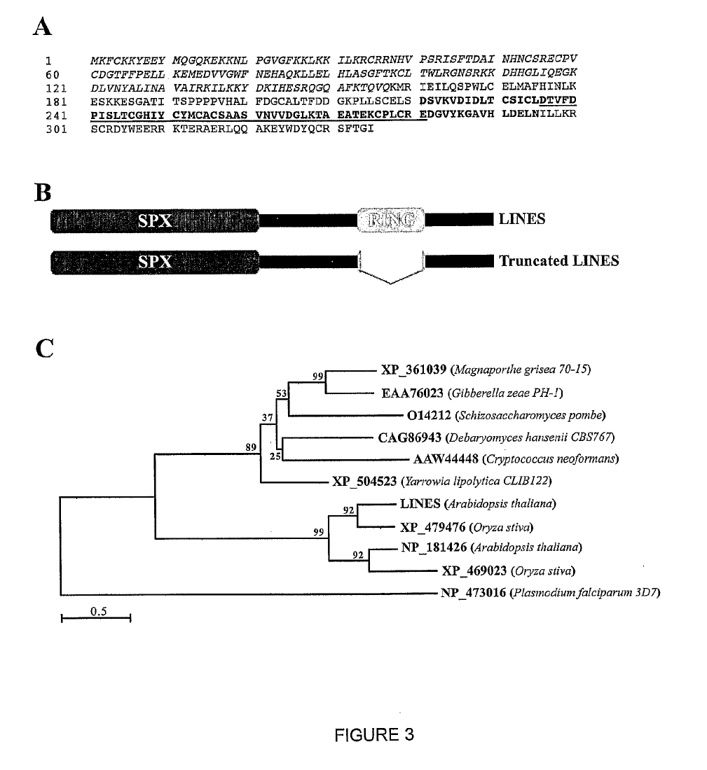

The LINES gene consists of six exons and five introns and encodes a protein of 335 amino acids (FIGS. 2A and 3A) with a molecular weight of 38,110 Dolton and a pl of 4.59. The LINES protein harbours two known domains, RING and SPX (FIGS. 3A and B). The RING domain is localized to the amino acids 230-282 in the LINES protein, and is a C3HC4 type Zn-finger which binds two atoms of zinc and may be involved in mediating protein-protein interactions. In Arabidopsis, functional characterization of some RING-containing proteins such as COP1 and SINATA5 suggests that the biological function of the RING domain is to participate in ubiquitin-dependent protein degradation (Moon et al, 2004), and thus plays a central and essential role in eukaryotic cellular regulation (Glickman and Ciechanover, 2002). Stone et al. (2005) reported that the Arabidopsis genome encodes 469 putative RING-containing proteins, which can be grouped into eight types, and LINES belongs to the RING-HCa type (Stone et al., 2005). The SPX domain resides at the N-terminus of LINES from amino acid 1 to 180 (FIGS. 3A and B). This domain was named after the yeast proteins SYGL and PHO81 and the mammalian protein XRP1, all of which contain this 180-amino-acid domain at their N-terminus. Although the exact biological function of the SPX domain is unknown, the finding that the N-terminus of yeast SYG1 can directly bind to the G-protein beta subunit and suppress the mating pheromone signal transduction (Spain et al., 1995) suggests that the proteins with N-terminal SPX domain may be involved in G-protein associated signal transduction.

Comparing the amino acid sequences from the mutated and wild type LINES proteins revealed that the RING domain was deleted in the mutated LINES protein (FIGS. 3A and B). Although the remaining amino acid sequence including the SPX domain of the truncated LINES is the same as that of the wild type counterpart, the truncated LINES could not execute its wild type counterpart's physiological function, and thus resulted in the low nitrogen induced early senescence phenotype. Further, to determine whether a wild type RING domain could rescue the lines' phenotype, the inventors expressed a N-terminal truncated At1g02860 cDNA which encodes the intact RING domain but no SPX in lines plants. However, all the transformants maintained the low nitrogen dependent early senescence phenotype (data not shown). These results indicate that the RING domain is a very essential part in LINES, and could not be separated from the SPX domain for its physiological function.

Although the Arabidopsis genome contains approximately 20 SPX-containing proteins (Wang et al., 2004) and 469 RING-harbouring proteins, only LINES and NP_{—}181426 encoded by At1g02860 and At2g38920, respectively, contain both SPX and RING domains. The two proteins share 41.2% sequence identity and 61.7% similarity. There is a T-DNA insertion mutant in At2g38920 (SALK_{—}129778). However, a homozygous mutation in this gene did not show the low nitrogen induced early senescence phenotype (data not shown), indicating that At1g02860 is specifically required by Arabidopsis plants to adapt to the nitrogen limitation growth condition. Comparing the deduced amino acid sequence of LINES with those deposited in the current database reveals LINES has two orthologs in rice (Oryza sativa), one in fission yeast (Schizosaccharomyces pombe), and six in Fungi (Figure). Phylogenetic analysis indicates that LINES is related most closely to XP_{—}479-476 in rice (FIG. 3C). However, the biological functions of all these LINES orthologs are still unknown.

### The Lines Mutant Did not Alter its Capacity for Acquiring Nitrogen but Instead Had an Altered Nitrogen Limitation Mediated Senescence Process

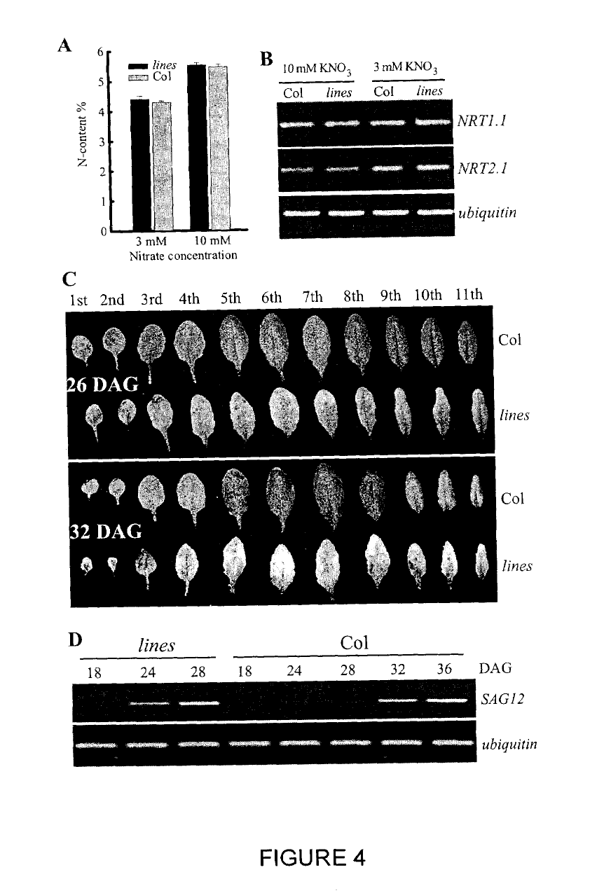

There are two possible reasons for the lines plants to develop the low inorganic nitrogen-induced early senescence phenotype. First, lines plants may acquire less nitrogen nutrient than wild type when nitrogen supply is insufficient. To address this issue, the inventors examined the total nitrogen contents in wild type and lines plants. Supplied with high (10 mM) or low (3 mM) nitrate, wild type and the lines plants at 18 DAG resembled each other in fresh weight (data not shown) and total nitrogen percent (FIG. 4A). Thus, the lines mutant closely resembles wild type with respect to total nitrogen content. Further, two major nitrate transporters, NRT1.1 (low nitrate affinity transporter) and NRT2.1 (high nitrate affinity transporter), had very similar expression levels in the roots of wild type and lines plants supplied with 3 or 10 mM nitrate (FIG. 4B). These results suggest that lines and wild type plants have similar capacity to acquire nitrogen nutrient whether the nitrogen supply is high or low.

The second possible reason for lines plants to produce the early senescence phenotype only when nitrogen supply is limited may be that lines plants are impaired in developing adaptive responses which enable Arabidopsis plants to acclimatize to the nitrogen limitation growth condition. One such adaptive response is nitrogen limitation mediated senescence, which is essential to remobilize the nitrogen nutrient from old, mature rosette leaves to young and active growing organs, such as young leaves and immature seeds (Thimann, 1980; Mei and Thimann, 1984). Therefore, the nitrogen limitation mediated senescence process in wild type and lines rosette leaves was examined in detail (FIG. 4C). Grown with 3 mM nitrate, wild type plants did not show senescence symptom in the 5^{th} and younger rosette leaves until 32 DAG. Senescence progressed slowly and was well organized, which was indicated by the fact that the rosette leaves gradually changed their colour from green to dark green and red, and kept their turgidity throughout the senescence process. In lines plants supplied with 3 mM nitrate, senescence not only started much earlier than that in wild type, but also progressed very rapidly because all the rosette leaves showed senescence symptoms at 26 DAG, and displayed abrupt leaf color change and swift leaf turgidity disappearance. The lower leaf blades of the senescing leaves were still green and turgid, while the upper parts already died (FIG. 4C). Further, the senescing leaves did not turn to red from green, and the dead leaves were brown with some redness, indicating little anthocyanin was synthesized during the senescence process in the lines plants.

Arabidopsis SAG12 is a senescence associated gene and has been defined as an authentic senescence molecular marker (Noh and Amasino, 1999). To make it more convenient and accurate to track the occurrence of senescence and sample rosette leaves for biochemical assays in this study, the inventors determined SAG12 expression in the 5^{th}-8^{th} rosette leaves of wild type and lines plants growing with 3 mM nitrate. As shown in FIG. 2D, SAG12 expression was detected in the lines plants at 24 days DAG and increased with the proceeding of senescence at 28 DAG. On the other hand, SAG12 in wild type plants was not expressed until 32 DAG, and increased markedly at 36 DAG when severe senescence occurred in the rosettes. The expression pattern of SAG12 gene was consistent with the appearance of the visual senescence symptom in the two genotypes (FIG. 4C), indicating that the SAG12 expression level indeed reflects the senescence process in the rosette leaves.

The Lines Mutant Contained High Levels of Nitrogen Metabolites while Failing to Accumulate Soluble Sugars in the Senescing Rosette Leaves

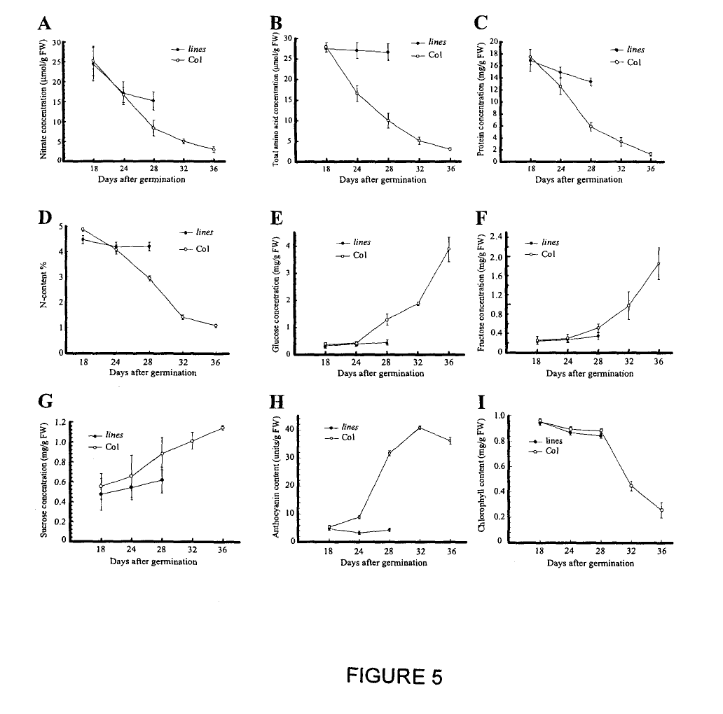

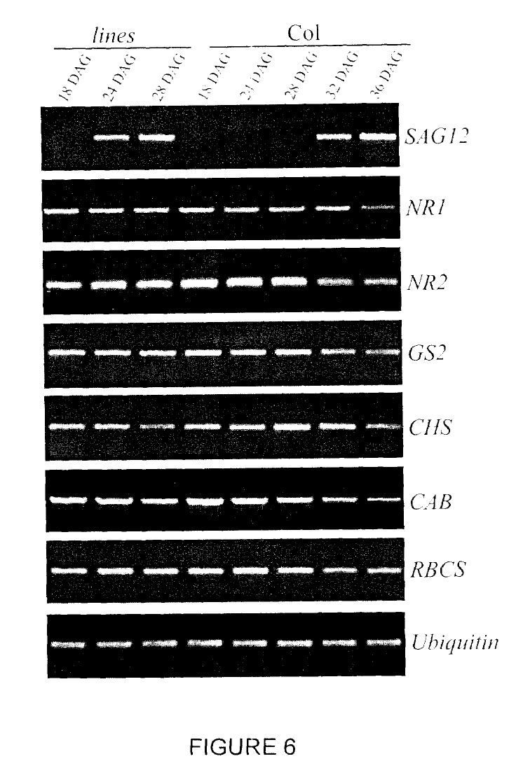

To make full use of the available nitrogen under conditions where it limits growth, plants can export nitrogen from old leaves and organs to young and developing ones. Therefore, the second nitrogen limitation adaptive response tested is the remobilization of nitrogen from senescent leaves. The rapid rosette leaf senescence and death in lines plants may impair the remobilization of nitrogen from these senescing leaves to young leaves, flowers and developing siliques, and thus may result in high nitrogen metabolite contents in the lines rosette leaves. To test this hypothesis, the levels of nitrate, amino acids, soluble proteins and total nitrogen content were determined in the 5^{th}-8^{th} rosette leaves of the wild type and lines plants grown under the limiting nitrogen condition throughout the senescence process. At 18 DAG, prior to the initiation of senescence, wild type and lines plants contained very similar amounts of the three N-containing compounds (FIG. 5A to C) and did not differ significantly in total nitrogen content (FIG. 5D). At 32 DAG when senescence occurred in wild type rosette leaves, the contents of nitrate, total amino acids, soluble proteins and total nitrogen were reduced by 75%, 80%, 75%, and 70%, respectively, as compared with those at 18 DAG when no senescence symptom was observed. With the progression of senescence in wild type rosette leaves at 36 DAG, the nitrate, total amino acids, soluble proteins and total nitrogen contents were decreased by 90%, 90%, 90%, and 80%, respectively (FIG. 5A to D). In contrast, the occurrence (at 24 DAG) and progression (at 28 DAG) of senescence in lines rosette leaves were not accompanied by a significant reduction in the amounts of nitrate, total amino acids, soluble proteins and total nitrogen (FIG. 5A to D). For example, when severe senescence occurred in lines rosette leaves at 28 DAG, the contents of nitrate, total amino acids, soluble proteins and total nitrogen were only decreased by 33%, 5%, 20% and 6%, respectively, as compared with those at 18 DAG (FIG. 5A to D). These results suggest that nitrogen is remobilized from senescing wild type rosette leaves, while nitrogen was maintained in lines senescing leaves. Following the biochemical analysis, the inventors determined the expression of the genes involved in nitrogen metabolism by RT-PCR. As shown in FIG. 6, the expression of NR1, NR2, and GS2 was decreased with the occurrence and proceeding of senescence in wild type rosette leaves. However, this did not occur in lines plants, where the expression of these genes was not altered between rosette leaves harvested either before or after senescence occurred.

Accumulation of soluble sugars, including glucose, fructose and sucrose in leaves has been found to be linked strongly to the occurrence of leaf senescence and nitrogen deficiency (Paul and Driscoll, 1997; Wingler et al., 2006). The assays for soluble sugars showed that glucose, fructose and sucrose accumulated markedly with the start and progress of leaf senescence in wild type plants, but the contents of the three soluble sugars in the lines plants only had a slight increase when senescence occurred in their rosette leaves (FIG. 5E to G). For example, at 18 DAG when leaf senescence had not been initiated, wild type and lines plants had similar contents of glucose, fructose and sucrose. When leaf senescence occurred, the amounts of glucose, fructose and sucrose increased 90%, 84%, and 46%, respectively, in wild type plants (at 36 DAG), while only increasing 5%, 8%, and 20%, respectively, in the lines plants (at 28 DAG).

### The Lines Mutant was Impaired in the Accumulation of Anthocyanin and the Reduction of Photosynthesis Capacity During Senescence

Accumulating anthocyanin and reducing photosynthesis are two important nitrogen limitation adaptive responses, and controlled by multiple QTLs in Arabidopsis (Diaz et al., 2006). To determine whether the lines plants could develop such adaptive responses, anthocyanin amounts and photosynthesis capacity were measured in wild type and lines plants. Wild type plants grown under limiting nitrogen increased anthocynanin content markedly during their growth and senescence (FIG. 5H). At 18 DAG, the wild type 5^{th}-8^{th} rosette leaves contained 4.8 units/g fresh weight (FW) anthocyanin, and this increased to 31 units/g FW at 28 DAG when rosette leaves still did not show any senescence symptoms. With the occurrence of senescence in the 5-8^{th} rosette leaves of wild type plants at 32 DAG, their anthocyanin content increased to >40 units/g FW. In contrast, anthocyanin accumulation did not occur in the senescing lines rosette leaves (FIG. 5H). At 18 DAG when the lines plants showed no senescence symptoms molecularly and morphologically, they contained 4.0 units/g FW anthocyanin. At 24 DAG when senescence occurred in the 5-8^{th} rosette leave, they still contained 3.5 units/g FW anthocyanin. When all the rosette leaves showed severe senescence symptom in the lines plants at 28 DAG, no increase in the anthocyanin content was observed (FIG. 5H). Corresponding to the different anthocyanin accumulation patterns in wild type and lines plants, the chalcone synthase gene (CHS), which encodes the rate limiting enzyme in the anthocyanin synthesis pathway, did not increase its transcription level throughout the senescence process in lines plants, while the expression of CHS gene increased markedly with the start and progress of senescence in wild type plants (FIG. 6).

Plant photosynthesis capacity can be indicated by the chlorophyll contents and the expression level of two photosynthesis marker genes RBCS and CAB in leaves, which encode the small subunit of Rubisco and the chlorophyll a/b binding protein, respectively (Martin et al., 2002). The chlorophyll content was assayed in wild type and lines plants supplied with limited nitrogen. As shown in FIG. 5I, wild type and lines plants contained 0.99 and 0.95 mg chlorophyll/g FW at 18 DAG, respectively. With the initiation (32 DAG) and progress (36 DAG) of senescence in wild type plants, the chlorophyll content decreased by 50% and 70%, respectively. However, the chlorophyll content was only reduced by 15% at 28 DAG when senescence occurred severely in the lines plants. RT-PCR analysis revealed that the expression of RBCS and CAB was reduced drastically when the low nitrogen mediated senescence occurred in wild type rosette leaves at 32 DAG (FIG. 6). In contrast, the expression of RBCS and CAB in lines plants was consistently high both before and after senescence in their rosette leaves (FIG. 6). These data indicate that unlike wild type plants, lines plants grown with limited nitrogen nutrient are impaired in the accumulation of anthocyanin and in the reduction of photosynthesis capacity, which are essential adaptive responses to nitrogen limitation.

### The Lines Mutant is not Defective in Phosphorus Limitation and High Sucrose Stress Induced Anthocyanin Synthesis Pathways

Besides nitrogen limitation, phosphorus limitation and high sucrose stress also induce anthocyanin synthesis in plants. To determine whether the lines mutant is defective in phosphorus limitation caused anthocyanin accumulation, the lines mutant and wild type plants were grown with 0.5 mM phosphorus in soil. This phosphorus application has been found to significantly limit Arabidopsis plant growth. At 28 DAG when the lines mutant plants grown with 3 mM nitrate already showed the early senescence phenotype and impairment in anthocyanin accumulation, both the lines mutant and wild plants supplied with 0.5 mM phosphorus strongly enhanced anthocyanin synthesis, and produced about 24 units/g FW anthocyanin. Further, the lines mutant plants grown with both limiting nitrate (3 mM) and phosphorus (0.5 mM) not only accumulated high amount anthocyanin (20 units/g FW) at 28 DAG, but also did not show the nitrogen limitation induced early senescence phenotype. To investigate whether high sucrose can induce anthocyanin accumulation in the lines mutant, both the lines mutant and wild type plants were grown in vitro with 1 mM nitrate, and 3% or 5% sucrose. At 21 DAG, the lines mutants supplied with 3% sucrose produced much less anthocyanin than did wild type, and displayed the early senescence phenotype. In contrast, grown with 5% sucrose, the lines plants accumulated the similar amount anthocyanin to wild type, and did not exhibit the early senescence phenotype. All the data indicate that the phosphorus limitation and high sucrose stress induced anthocyanin synthesis pathways are not affected by the lines mutation, and the lines mutant is specifically defective in the nitrogen limitation induced anthocyanin pathway. These results also demonstrate that accumulation of anthocyanin in the lines mutant can effectively prevent the nitrogen limitation induced early senescence phenotype.

### The Lines Mutation Switched Nitrogen Limitation Enhanced Anthocyanin Synthesis to Lignin Production

The phenylpropanoid pathway in plants divides into two branches at the fourth step: one is for flavonoid biosynthesis, and the other for lignin biosynthesis. In the lines mutant plants grown with 3 mM nitrate, many structural genes involved in lignin synthesis were up-regulated significantly from 22 to 28 DAG. This result and the marked decrease of nitrogen limitation induced anthocyanin accumulation in the lines mutant plants suggest that the lines mutation may switch nitrogen limitation enhanced anthocyanin synthesis to lignin production. To confirm this hypothesis, the lines mutant and wild type plants were grown with 3 mM nitrate, and the lignin contents in the primary inflorescences were analyzed from 22 to 28 DAG. At 22 DAG when both the lines mutant and wild type plants have 1-2 cm inflorescence, the wild type plants contained almost no lignin, while lignin was obviously observed in the lines mutant. At 24 DAG before the nitrogen limitation induced early senescence occurred, the lines mutant plants still had much more lignin than did wild type. With the early senescence initiated and developed, the lines mutant plants decreased their growth, and had similar lignin content to wild type at 28 DAG. To maintain the growth of the lines mutant plants under a nitrogen limitation growth condition, they were supplied with high carbon dioxide (CO₂). Under this growth condition, although the lines plants exhibited early senescence phenotype and produced little anthocyanin at 28 DAG, they maintained their growth and continued accumulating lignin in the stems, which results in that the lines mutant plants contained much more lignin and had much taller statute than did wild type. All these results indicate that the LINES gene is involved in controlling lignin biosynthesis, and the lines mutation switches nitrogen limitation enhanced anthocyanin synthesis to lignin production.

### Discussion

Both nitrogen and phosphorous are essential macronutrients for plant growth and development (Marschner, 1995). In contrast with the plant response to nitrogen limitation, the phosphorous limitation sensing, signaling and associated gene regulation has been well investigated and understood in plants. In Arabidopsis, mutations in the PHO3, PSR1, PDR2 and PHR1 genes disrupted the phosphorous limitation signalling (Zakhleniuk et al., 2001; Chen et al., 2000, Ticconi et al., 2004; Rubio et al., 2001). AtSIZ1 regulates the activity of PHR1, and may act downstream of the phosphorous limitation sensing pathway (Miura et al., 2005). The evidence for the existence of the nitrogen limitation sensing and signaling pathway in living organisms comes from yeast and bacteria. In yeast, the ammonium permease Mep2p can sense the nitrogen availability in the environment and generate the nitrogen limitation signal for yeast to regulate its growth and development to acclimatize to the unfavourable growth condition (Lorenz and Heitman, 1998; Gagiano et al., 2002; Biswas and Morschhäuser, 2005). In bacteria, the PII-type signal transduction proteins have been found to play a central role in the nitrogen limitation signaling pathway with their function and interaction with other proteins being modified by phosphorylation, uridylylation or adenylylation in response to the intracellular nitrogen status (deficient or sufficient) (Arcondeguy et al., 2001; Schwarz and Forchhammer, 2005). Although plants have the homologues of yeast Mep2p and bacterial PII, they do not have a similar function in nitrogen limitation adaptability in plants (Crawford and Forde, 2002; Moorhead and Smith, 2003). In this study, the inventors present physiological, biochemical and molecular genetic data to clearly demonstrate that LINES is required for the development of the nitrogen limitation adaptive responses, and is an essential component in the molecular mechanism governing plant nitrogen limitation adaptability.

Nitrogen limitation stress has been known to markedly affect plant growth and development, while the knowledge about plant adaptability to nitrogen limitation is obscure. In this study, the inventors demonstrate that Arabidopsis plants are able to acclimatize to the nitrogen limitation growth condition by developing a set of nitrogen limitation adaptive responses (FIGS. 4 and 5). First, the photosynthesis capacity was reduced. This response can not only markedly decrease plants' requirement for the nitrogen nutrient, and also restrict the utilization of photosynthates in synthesizing N-containing molecules, such as amino acids, proteins and nucleic acids. Second, the synthesis of anthocyanin was significantly increased. The increase in this light protecting pigment allows the nitrogen deficient plants to avoid photoinhibition damages caused by nitrogen limitation (Bongue-Bartelsman and Phillops, 1995; Chalker-Scott, 1999). Third, soluble sugars which may function as the signal for leaf senescence and nitrogen deficiency were accumulated (Paul and Driscoll, 1997; Wingler et al., 2006). Leaf senescence has been known to be important for nitrogen recycling in Arabidopsis because more than 80% of the total nitrogen in mature rosette leaves is exported through the senescence process (Himelblau and Amasino, 2001). Under the nitrogen limitation growth condition, this senescence mediated nitrogen recycling becomes more important for Arabidopsis plants to efficiently use the limited nitrogen nutrient, and thus is an essential nitrogen limitation adaptive response. In this study, Arabidopsis plants grown with limited nitrogen supply started senescence in old mature rosette leaves after entering the bolting stage, and senescence progressed gradually from the older mature rosette leaves to younger ones (FIG. 4). Concomitantly, the total nitrogen content and the amounts of N-containing compounds such as proteins, amino acids and chlorophyll decreased markedly in the senescing rosette leaves, indicating nitrogen in these leaves was exported to young, developing organs such as floral buds and siliques (FIG. 5). Correspondingly, with the growth and progress of nitrogen limitation caused senescence in Arabidopsis plants, the expression levels of the genes involved in photosynthesis and nitrogen assimilation were decreased, and the transcription of CHS, the rate-limiting gene in anthocyanin synthesis, was enhanced (FIG. 6). These numerous physiological, biochemical and molecular changes involved in the nitrogen limitation adaptive responses in Arabidopsis plants suggest that a sensing or signaling pathway for nitrogen limitation is activated to initiate such a complicated metabolic response to low nitrogen.

The lines mutant only develops the early senescence phenotype when grown under limiting nitrogen, and this phenotype was confirmed in three ways. First, the lines plants grown under limited nitrogen (3 mM nitrate) started senescence in rosette leaves at least one week earlier than did wild type plants, while the lines plants supplied with sufficient nitrogen nutrient (10 mM nitrate) were similar to wild type in every aspect of growth and development throughout their life cycles (FIG. 1A to C). Second, supplying sufficient nitrogen nutrient (15 mM nitrate) to the senescing lines plants could stop the senescence program (FIGS. 1F and G). Third, the lines mutant does not show the early senescence phenotype when exposed to conditions of low phosphorus or high sucrose in combination with low nitrogen.

The appearance of the early senescence phenotype in lines plants under nitrogen limitation could be explained either by a defect in nitrogen acquisition, or by the loss of adaptability to the nitrogen limitation growth condition. The first possibility was excluded since the lines and wild type plants had very similar total nitrogen contents when either 3 mM or 10 mM nitrate was supplied (FIG. 4A). On the other hand, this detailed analysis of the lines mutant clearly demonstrated that all the physiological, biochemical and molecular changes essential for nitrogen limitation adaptive responses failed to occur in the lines plants supplied with limited nitrogen (FIG. 4 to 6). From the late vegetative to the reproductive stage, the lines plants grown with limited nitrogen supply did not accumulate anthocyanin, and only had a slight reduction in photosynthesis and little increase in soluble sugar content (FIG. 5). Interestingly, the inability to accumulate anthocyanin is accompanied by increased lignin accumulation, which suggests that LINES regulates the phenylpropanoid biosynthesis branch point between lignin production and anthocyanin production.

The most salient feature of these lines plants was that they went through a nitrogen limitation caused senescence pathway strikingly different from that in wild type. First, senescence in the lines plants supplied with limited nitrogen not only started much earlier and progressed more rapidly in all rosette leaves than that found in wild type, but this also occurred abruptly in the young, active growing organs such as cauline leaves and immature siliques (FIG. 4). Second, the total nitrogen content and the N-containing compounds such as proteins and total amino acids in the senescing lines leaves were only slightly reduced with the initiation and progress of senescence in the lines rosette leaves (FIG. 5), indicating that senescence mediated nitrogen remobilization did not occur in the senescing lines rosette leaves. This was also manifested by the rapid senescence in the lines' young rosette and cauline leaves, and developing siliques, which is most likely due to lower nitrogen importation. Further, the genes involved in photosynthesis, nitrogen assimilation and anthocyanin synthesis did not have altered expression levels during senescence in the lines plants. This was significantly different from what was seen in wild type plants (FIG. 6).

The failure of the lines mutant to develop these essential nitrogen limitation adaptive responses strongly suggests that the mutation in LINES gene disrupts the nitrogen limitation sensing or signaling pathway, so that the lines mutant can not sense or signal the nitrogen limitation growth condition. Instead the lines mutant maintains a physiological, biochemical and molecular status as though the nitrogen supply is not limiting.

The LINES gene was identified by a map-based cloning approach and shown to encode a RING-type ubiquitin ligase associated with SPX domain (FIGS. 2 and 3). In the lines mutant, the RING domain was deleted from the LINES protein, and the truncation of LINES caused the low nitrogen induced early senescence phenotype (FIG. 2B). A mutant with a T-DNA insertion in At2g38920, which is the sole homolog of LINES in Arabidopsis genome, did not show the lines phenotype when supplied with insufficient nitrogen, further indicating that LINES is specifically important for the development of nitrogen limitation adaptive responses in Arabidopsis. However, the absence of a phenotype may reflect redundancy conferred by LINES.

Protein ubiquitination has been known to play central roles in regulating numerous cellular processes in eukaryotes. First, protein ubiquitination pathway targets various substrates such as nuclear transcription factors, abnormal cytoplasmic proteins, and short-lived regulatory proteins for degradation by the 26S proteasome (Glickman and Ciechanover, 2002). Second, modification of proteins with ubiquitin also regulates protein localization, activity, interacting partners, and functions in a proteasome-independent manner (Schnell and Hicke, 2003; Sun and Chen, 2004). Characterization of the lines mutant in this study demonstrated that the development of nitrogen limitation adaptive responses in Arabidopsis involved numerous physiological, biochemical and molecular changes (FIG. 4 to 6), for which the responsible cellular process such as nitrogen limitation sensing and signaling should be activated. Based on the finding here that LINES encodes an ubiquitin ligase and the knockout of LINES resulted in the failure to develop all essential nitrogen limitation adaptive responses in the lines mutant, it can be hypothesized that LINES may be involved in the degradation or modification of substrate protein(s) via the protein ubiquitination pathway, and the substrate protein(s) may be the key negative regulator(s) in the nitrogen limitation sensing or signaling pathway. In the lines mutant, this negative regulator(s) would not be properly ubiquitinated for degradation or modification because of the deletion of RING domain from LINES.

Crops such as maize with a strong adaptability to nitrogen limitation are most desirable in developing countries in Latin America, Africa and Asian, where farmers can not afford to the large nitrogen input while the food requirement is very high (Loomis, 1997; Duvick, 1997). Understanding the molecular mechanism controlling plant adaptability to nitrogen limitation will hopefully accelerate the development of such crop cultivars. The cloning and functional characterization of LINES in this study not only demonstrates that plants are equipped with a molecular mechanism to adapt to nitrogen limitation, but also is the first step in the identification of the molecular components involved in controlling plant nitrogen limitation sensing, signalling, and associated gene regulation.

Having now described particular embodiments of the invention by way of the foregoing examples, which are not intended to be limiting, the invention will now be further set forth in the following claims. Those skilled in the art will recognize that the claims also permit for the inclusion of equivalents beyond the claims' literal scope.

### REFERENCES

Alonso, J. M., et al. (2003) Genome-wide insertional mutagenesis of Arabidopsis thaliana. Science 301, 653-657.
Arcondeguy, T., Jack, R. and Merrick, M. (2001). P_{II} signal transduction proteins, pivotal players in microbial nitrogen control. Microbiol. Mol. Biol. Rev. 65, 80-105.
Amon, D. I. (1949) Copper enzymes in isolated chloroplasts. Polyphenoloxidase in Beta vulgaris. Plant Physiol. 24: 1-15.
Bi, Y. M. Zhang, Y. Signorelli, T., Zhao, R., Zhu, T., and Rothstein, S. J. (2005) Genetic analysis of Arabidopsis GATA transcription factor gene family reveals a nitrate-inducible member important for chlorophyll synthesis and glucose sensitivity. Plant J. 44: 680-92.
Biswas, K., and Morschhäuser, J. (2005) The Mep2p ammonium permease controls nitrogen starvation-induced filamentous growth in Candida albicans. Mol. Microbiol. 56:3, 649-669.
Bongue-Bartelsman, M. and Phillips, D. A. (1995) Nitrogen stress regulates gene expression of enzymes in the flavonoid biosynthetic pathway of tomato. Plant Physiol. Biochem. 33: 539-546.
Castleberry, R. M., Crum, C. W. and Krull, F. (1984) Genetic yield improvement of U.S. maize cultivars under varying fertility and climatic environments. Crop Sci. 24: 33-36.
Cataldo, D. A., Haroon, M., Schrader, T. E. and Youngs, V. L. (1975) Rapid colorimetric determination of nitrate in plant tissue by nitration of salicylic acid. Commun. Soil Sci. and Plant Anal. 6: 71-80.
Chalker-Scott, L. (1999) Environmental significance of anthocyanins in plant stress responses. Photochem. Photobiol. 70:1-9.
Chen, D. L., Delatorre, C. A., Bakker, A. and Abel, S. (2000) Conditional identification of phosphate-starvation-response mutants in Arabidopsis thaliana. Planta 211: 13-22.
Clougli, S. J. and Bent, A. F. (1998) Floral dip: a simplified method for Agrobacterium-mediated transformation of Arabidopsis thaliana. Plant J. 16: 735-743.
Crawford, N. M. (1995) Nitrate: nutrient and signal for plant growth. Plant Cell 7: 859-68.
Crawford, N. M. and Forde, B. G. (2002) Molecular and developmental biology of inorganic nitrogen nutrition. In: Meyerowitz, E. and Somerville, C. eds, The Arabidopsis Book. American Society of Plant Biologists, Rockville, Md., doi/10.1199/tab.0011, http://www.aspb.org/publicatications/arabidopsis
Cutler, S. R., Ehrhardt, D. W., Griffitts, J. S, and Somerville, C. R. (2000) Random GFP::cDNA fusions enable visualization of subcellular structures in cells of Arabidopsis at a high frequency. Proc. Natl. Acad. Sci. U.S.A. 97: 3718-3723.
Diaz, C., Purdy, S., Christ, A., Morot-Gaudry, J.-F., Wingler, A. and Masclaux-Daubresse, C. (2005) Characterization of markers to determine the extent and variability of leaf senescence in Arabidopsis. A metabolic profiling approach. Plant Physiol. 138: 898-908.
Diaz, C., Saliba-Colombani. V., Loudet, O., Belluoino, P., Moreau, L., Daniel-Vedele, F., Morot-Gaudry, J. F. and Masciaux-Daubresse, C. (2006) Leaf yellowing and anthocyanin accumulation are two genetically independent strategies in response to nitrogen limitation in Arabidopsis thaliana. Plant Cell Physiol. 47: 74-83.
Ding, L., Wang, K. J., Jiang, G. M., Biswas, D. K., Xui, H., Li, L. F. and Li Y. H. (2005) Effects of nitrogen deficiency on photosynthetic traits of maize hybrids released in different years. Ann Bot (Lond.). 96: 925-930.
Duvick, D. N. (1984) Genetic contributions to yield gains of U.S. hybrid maize, 1930 to 1980. In: Fehr, W. R., ed. Genetic contributions to yield gains of five major crop plants. CSSA Special Publication 7. Madison, Wis.: ASA and CSSA, 15-47.
Duvick, D. N. (1997) Review of the symposium on developing drought and low-N tolerant maize. In: Edmeades, G. O., Banziger, M., Mickelson, H. R. and Pena-Valdicia, C. B. eds. Developing drought and low N-tolerant maize. El Batan, Mexico: CIMMYT, 554-556.
Feller, U. and Fischer, A. (1994) Nitrogen metabolism in senescing leaves. CRC Crit. Rev. Plant Sci. 13: 241-273.
Filleur, S., Dorbe, M. F., Cerezo, M., Orsel, M., Granier, F., Gojon, A., and Daniel-Vedele, F. (2001). An arabidopsis T-DNA mutant affected in Nrt2 genes is impaired in nitrate uptake. FEBS Lett. 489: 220-224.
Frink, C. R., Waggoner, P. E., and Ausubel, J. H. (1999) Nitrogen fertilizer: retrospect and prospect, Proc. Natl. Acad. Sci. U.S.A. 96: 1175-1180.
Gagiano, M., Bauer, F. F., and Pretorius, J. S. (2002) The sensing of nutritional status and the relationship to filamentous growth in Saccharomyces cerevisiae. FEMS Yeast Res. 2: 433-470.
Geiger, M., Walch-Liu, P., Engels, C., Harnecker, J., Schulze, E.-D., Ludewig, F., Sonnewald, U., Scheible, W.-R., and Stitt, M. (1998) Enhanced carbon dioxide leads to a modified diurnal rhythm of nitrate reductase activity in older plants, and a large stimulation of nitrate reductase activity and higher levels of amino acids in young tobacco plants. Plant, Cell & Environ. 21: 253-268.
Glickman, M. H. and Ciechanover, A. (2002) The ubiquitin-proteasome proteolytic pathway: destruction for the sake of construction. Physiol Rev. 82: 373-428
Good, A. G., Shrawat, A. K. and Muench, D. G. (2004) Can less yield more? Is reducing nutrient input into the environment compatible with maintaining crop production? Trends Plant Sci. 9: 597-605.
Himelblau, E. and Amasino, R. M. (2001) Nutrients mobilized from leaves of Arabidopsis thaliana during leaf senescence. J. Plant Physiol. 158: 1317-1323.
Khamis, S., Lamaze, T., Lemoine, Y. and Foyer, C. (1990) Adaptation of the photosynthetic apparatus in maize leaves as a result of nitrogen limitation. Plant Physiol. 94: 1436-1443.
Kolber, Z., Zehr, J. and Falkowski, P. (1988) Effects of Growth Irradiance and Nitrogen Limitation on Photosynthetic Energy Conversion in Photosystem II. Plant Physiol. 88: 923-929.
Lam, H. M. Coschigano, K. T., Oliveira, I. C., Melo-Oliveira. R., Coruzzi G. M. (1996) The Molecular-genetics of nitrogen assimilation into amino acids in higher plants. Annu. Rev. Plant Physiol. Plant Mol. Biol. 47: 569-593.
Loomis, R. S. (1997) Developing drought and low-nitrogen tolerant maize: an overview. In: Edmeades, G. O., Banziger, M., Mickelson, H. R. and Pena-Valdicia, C. B. eds. Developing drought and low N-tolerant maize. El Batan, Mexico: CIMMYT, 552-555.
Lukowitz, W., Gillmor, C. S. and Scheible, W. (2000) Positional cloning in Arabidopsis. Why it feels good to have a genome initiative working for you. Plant Physiol. 123, 795-805.
Marschner, H. (1995). Mineral nutrition of higher plants. London: Academic press.
Martin, T., Oswald, O. and Graham, I. A. (2002) Arabidopsis seedling growth, storage lipid mobilization, and photosynthetic gene expression are regulated by carbon:nitrogen availability. Plant Physiol. 128: 472-481 McCullough D. E., Girardin, Ph., Mihajlovic, M., Aguilera, A. and Tollenaar, M. (1994) Influence of N supply on development and dry matter accumulation of an old and a new maize hybrid. Can. J. Plant Sci. 74:471-477. Masclaux, C., Valadier, M., Brugiere, N., Morot-Gaudry, J. and Hirel, B. (2000) Characterization of the sink/source transition in tobacco (Nicotiana tabacum L.) shoots in relation to nitrogen management and leaf senescence. Planta 211: 510-518.
Mei, H. S. And Thimann, K. V. (1984) The relation between nitrogen deficiency and leaf senescence. Physiol. Plant. 62: 157-161.
Miura, K., Rus A., Sharkhuu, A. Yokoi, S., Karthikeyan, A. S., Raghothama, K. G., Baek. D. Koo, Y. D., Jin, J. B., Bressan, R. A., Yun, D. J. and Hasegawa, P. M. (2005) The Arabidopsis SUMO E3 ligase SIZ1 controls phosphate deficiency responses. Proc. Natl. Acad. Sci. USA. 102: 7760-7765.
Moon, J., Parry, G. and Estelle, M. (2004) The ubiquitin-proteasome pathway and plant development. Plant Cell 16: 3181-95.
Moorhead, G. B. and Smith. C. S. (2003) Interpreting the plastid carbon, nitrogen, and energy status. A role for PII? Plant Physiol. 133: 492-492.
Noh, B. and Spalding, E. P. (1998) Anion channels and the stimulation of anthocyanin accumulation by blue light in Arabidopsis seedlings. Plant Physiol. 116: 503-9.
Noh, Y. S, and Amasino, R. M. (1999) Identification of a promoter region responsible for the senescence-specific expression of SAG12. Plant Mol. Biol. 41: 181-94.
Nolden, L., Ngouoto-Nkili, C. E., Bendt, A. K., Kramer, R. and Burkovski, A. (2001) Sensing nitrogen limitation in Corynebacterium glutamicum: the role of glnK and glnD. Mol. Microbiol. 42:1281-95.
Ono, K., Terashima, I. and Watanabe, A. (1996) Interaction between nitrogen deficit of a plant and nitrogen content in the old leaves. Plant and Cell Physiol. 37: 1083-1089.
Paul, M. J. and Driscoll, S. P. 1997. Sugar repression of photosynthesis: the role of carbohydrates in signalling nitrogen deficiency through source:sink imbalance. Plant, Cell & Environ. 20: 110-116.
Peoples, M. B. Gault, R. R., Lean, B., Sykes, J. D. and Brockwell, J. (1995) Minimizing gaseous losses of nitrogen. In: Bacon, PE. ed, Nitrogen Fertilizer in the Environment, Marcel Dekker, pp. 565-606.
Rogers, A., Fischer, B. U., Bryant, J., Frehner, M., Blum, H., Raines, C. A. and Long, S. P. (1998). Acclimation of photosynthesis to elevated CO₂ under low-nitrogen nutrition is affected by the capacity for assimilate utilization. Perennial ryegrass under free-air CO₂ enrichment. Plant Physiol. 118: 683-689.
Rosen, H. (1957) A modified ninhydrin calorimetric analysis for amino acids. Arch. Biochem. Biophy. 67: 10-15.
Rubio, V., Linhares, F., Solano, R., Martin, A. C., Iglesias, J., Leyva, A. and Pa-Ares, J. (2001) A conserved MYB transcription factor involved in phosphate starvation signaling both in vascular plants and in unicellular algae. Genes Dev. 15: 2122-2133.
Schnell, J. D. and Hicke, L. (2003) Non-traditional functions of ubiquitin and ubiquitin-binding proteins. J. Biol. Chem. 278: 35857-35860
Spain, B. H., Koo, D., Ramakrishin, M., Dzudzor, B. and Colicelli, J. (1995) Truncated forms of a novel yeast protein suppress the lethality of a G protein alpha subunit deficiency by interacting with the beta subunit. J. Biol. Chem. 270: 25435-44.
Stitt, N. (1999) Nitrate regulation of metabolism and growth. Curr. Opin. Plant Biol. 2: 178-86.
Stone, S. L., Hauksdottir, H., Troy, A., Herschleb, J., Kraft, E. and Callis, J. (2005) Functional analysis of the RING-type ubiquitin ligase family of Arabidopsis. Plant Physiol. 137: 13-30.
Sun, L. and Chen, Z. J. (2004) The novel functions of ubiquitination in signaling. Curr. Opin. Cell Biol. 16: 119-126.
Thimann, K. V. (1980) Senescence in leaves. In: Thimann, K. V. ed. Senescence in Plants, C.R.C. Press, Boca Ration, Fla. 85-115.
Ticconi, C. A., Delatorre, C. A., Lahner, B., Salt, D. E. and Abel, S. (2004) Arabidopsis pdr2 reveals a phosphate-sensitive checkpoint in root development. Plant J. 37: 801-814.
Todd, C. D., Zeng, P., Huete, A. M. Hoyos. M. E. and Polacco, J. C. (2004) Transcripts of MYB-like genes respond to phosphorous and nitrogen deprivation in Arabidopsis. Planta 219: 1003-1009.
Tollenaar, M. and Wu, J. (1999) Yield improvement in temperate maize is attributable to greater stress tolerance. Crop Sci. 39: 1597-1604
Van Nuland, A., Vandormael, P., Donaton, M., Alenquer, M., Lourenço, A., Quintino, E., Versele, M. and Thevelein, J. M. (2006) Ammonium permease-based sensing mechanism for rapid ammonium activation of the protein kinase A pathway in yeast. Mol. Microbiol. 59: 1485-1505.
Vitousek, P. M. and Howarth, R. V. (1991) Nitrogen limitation on land and in the sea: how can it occur? Biogeochemistry 13: 85-115.
Wang, Y., Ribot, C., Rezzonico, E. and Poirier, Y. (2004) Structure and expression profile of the Arabidopsis PHO1 gene family indicates a broad role in inorganic phosphate homeostasis. Plant Physiol. 135: 400-11.
Wingler, A., Purdy, S. MacLean, J. A. and Pourtau, N. (2006) The role of sugars in integrating environmental signals during the regulation of leaf senescence. J. Exp. Bot. 57: 391-9.
Zakhleniuk, O. V., Raines, C. A. and Lloyd, J. C. (2001) pho3: a phosphorus-deficient mutant of Arabidopsis thaliana (L.) Heynh. Planta 212: 529-534.

1. A method of modulating a characteristic in a plant cell comprising modulating expression of a RING-like ubiquitin E3 ligase gene in the plant cell.

2. The method according to claim 1, wherein the expression of the RING-like ubiquitin E3 ligase gene is modulated by administering, to the cell, an effective amount of an agent that can modulate the expression levels of a RING-like ubiquitin E3 ligase gene in the plant cell.

3. The method according to claim 1, wherein the characteristic is an agronomic trait.

4. The method according to claim 3, wherein the characteristic is one that is affected by nitrogen, carbon and/or sulfur metabolism, biosynthesis of lipids, perception of nutrients, nutritional adaptation, electron transport and/or membrane associated energy conservation.

5. The method according to claim 3, wherein the characteristic is selected from one or more of nitrogen utilization, yield, cell growth, reproduction, photosynthesis, nitrogen assimilation, disease resistance, differentiation, signal transduction, lignin biosynthesis, anthocyanin biosynthesis, gene regulation, abiotic stress tolerance and nutritional composition.

6. The method according to claim 5, wherein the characteristic is nitrogen utilization.

7. The method according to claim 5, wherein the characteristic is yield.

8. The method according to claim 5, wherein the characteristic is lignin biosynthesis.

9. The method according to claim 5, wherein the characteristic is anthocyanin biosynthesis.

10. The method according to claim 1, wherein the plant cell is a dicot, a gymnosperm or a monocot.

11. The method according to claim 10, wherein the plant cell is a dicot.

12. The method according to claim 10, wherein the monocot is selected from the group consisting of maize, wheat, barley, oats, rye, millet, sorghum, triticale, secale, einkom, spelt, emmer, teff, milo, flax, gramma grass, Tripsacum sp. and teosite.

13. The method according to claim 11, wherein the dicot is selected from the group consisting of soybean, tobacco and cotton.

14. The method according to claim 2, wherein the agent enhances the expression levels of a RING-like ubiquitin E3 ligase gene in the plant cell.

15. The method according to claim 14, wherein the modulated characteristic is an increase or improvement in one or more of nitrogen utilization, yield, cell growth, reproduction, photosynthesis, nitrogen assimilation, anthocyanin biosynthesis, disease resistance, differentiation, signal transduction, gene regulation, abiotic stress tolerance and nutritional composition.

16. The method according to claim 14, wherein the agent that enhances the expression levels of a RING-like ubiquitin E3 ligase gene in the plant cell comprises a nucleic acid molecule encoding a RING-like ubiquitin E3 ligase.

17. The method according to claim 16, wherein the nucleic acid molecule comprises the sequence of SEQ ID NO:1, SEQ ID NO:5, SEQ ID NO:7, SEQ ID NO:9, or a functional fragment thereof.

18. The method according to claim 16, wherein the nucleic acid molecule comprises a sequence that hybridizes under medium stringency conditions to the nucleotide sequence of SEQ ID NO:1 or a functional fragment thereof.

19. The method according to claim 16, wherein the nucleic acid molecule comprises a nucleic acid sequence derived from the nucleotide sequence of SEQ ID NO:1, SEQ ID NO:5, SEQ ID NO:7, or SEQ ID NO:9, and has a nucleotide sequence comprising codons specific for expression in plants.

20. The method according to claim 16, wherein the nucleic acid molecule encodes a polypeptide listed in SEQ ID NO:2, SEQ ID NO:6, SEQ ID NO:8, or SEQ ID NO:10.

21. The method according to claim 2, wherein the agent that can modulate the expression levels of a RING-like ubiquitin E3 ligase gene in a plant cell comprises:
(a) a nucleotide sequence of SEQ ID NO:1 or a fragment or domain thereof,
(b) a nucleotide sequence encoding a polypeptide of SEQ ID NO:2, a fragment or domain thereof,
(c) a nucleotide sequence having substantial similarity to (a) or (b);
(d) a nucleotide sequence capable of hybridizing to (a), (b) or (c);
(e) a nucleotide sequence complementary to (a), (b), (c) or (d); or
(f) a nucleotide sequence that is the reverse complement of (a), (b), (c) or (d).

22. The method according to claim 2, wherein the agent that can modulate the expression levels of a RING-like ubiquitin E3 ligase gene in a plant cell comprises:
(a) a polypeptide sequence listed in SEQ ID NO:2, or a functional fragment, domain, repeat, or chimera thereof,
(b) a polypeptide sequence having substantial similarity to (a),
(c) a polypeptide sequence encoded by a nucleotide sequence identical to or having substantial similarity to a nucleotide sequence listed in SEQ ID NO:1, or a functional fragment or domain thereof, or a sequence complementary thereto; or
(d) a polypeptide sequence encoded by a nucleotide sequence capable of hybridizing under medium stringency conditions to a nucleotide sequence listed in SEQ ID NO:1, or to a sequence complementary thereto.

23. The method according to claim 16, wherein the nucleic acid sequence is expressed in a specific location or tissue of the plant.

24. The method according to claim 23, wherein the location or tissue is selected from one or more of seed, epidermis, root, vascular tissue, meristem, cambium, cortex, pith, leaf and flower.

25. The method according to claim 24, wherein the location or tissue is a seed.

26. The method according to claim 16, wherein the agent that enhances the expression levels of a RING-like ubiquitin E3 ligase gene in the plant cell comprises an expression cassette for modulating a characteristic in a plant cell including a promoter sequence operably linked to the isolated nucleic acid encoding a RING-like ubiquitin E3 ligase.

27-31. (canceled)

32. An expression cassette comprising a promoter sequence operably linked to an isolated nucleic acid molecule according to any one of SEQ ID NOs:1, 3, 5, 7 or 9, or a fragment or variant thereof.

33. A vector comprising a nucleic acid molecule according to any one of SEQ ID NOs:1, 3, 5, 7 or 9, or a fragment or variant thereof.

34. A plant cell transformed with a nucleic acid according to any one of SEQ ID NOs:1, 3, 5, 7 or 9, or a fragment or variant thereof.

35. A method of producing a transgenic plant comprising:
(1) providing an isolated nucleic acid according to any one of SEQ ID NOs:1, 3, 5, 7 or 9, or a fragment or variant thereof, and
(2) introducing the nucleic acid into the plant, wherein the nucleic acid is expressed in the plant.

36. The method according to claim 35 wherein the nucleic acid is SEQ ID NO:1 and the plant demonstrates an increase or improvement in one or more of nitrogen utilization, yield, cell growth, reproduction, photosynthesis, nitrogen assimilation, disease resistance, differentiation, anthocyanin biosynthesis, signal transduction, gene regulation, abiotic stress tolerance and nutritional composition.

37. The method of claim 35 wherein the nucleic acid is introduced into the plant using a method selected from the group consisting of microparticle bombardment, Agrobacterium-mediated transformation, and whiskers-mediated transformation.

38. A plant produced using the method of claim 35.

39. Seed of the plant of claim 38.

40. A plant cell of the plant of claim 38.

41-42. (canceled)

43. An antibody raised against an isolated polypeptide comprising:
(a) a polypeptide sequence of SEQ ID NO:2, SEQ ID NO:4, SEQ ID NO:6, SEQ ID NO:8, or SEQ ID NO:10, or a fragment, domain, repeat or chimera thereof;
(b) a polypeptide sequence having substantial similarity to (a);
(c) a polypeptide sequence encoded by a nucleotide sequence identical to or having substantial similarity to a nucleotide sequence listed in SEQ ID NO:1, SEQ ID NO:3, SEQ ID NO:5, SEQ ID NO:7, SEQ ID NO:9, or a fragment or domain thereof, or a sequence complementary thereto;
(d) a polypeptide sequence encoded by a nucleotide sequence capable of hybridizing under medium stringency conditions to a nucleotide sequence listed in SEQ ID NO:1, SEQ ID NO:3, SEQ ID NO:5, SEQ ID NO:7, or SEQ ID NO:9, or to a sequence complementary thereto; or
(e) a functional fragment of (a), (b), (c) or (d).

44. The antibody according to claim 43 wherein the polypeptide comprises the sequence of SEQ ID NO:2, SEQ ID NO:4, SEQ ID NO:6, SEQ ID NO:8 or SEQ ID NO:10 or a variant thereof having a conservative amino acid modification.

45. An immunoassay kit comprising the antibody of claim 43 and instructions for the use thereof.

46-56. (canceled)


SEQUENCE LISTING (12 sequences)

SEQ ID NO: 1 - DNA, 1008 bp, Arabidopsis
atgaagttttgtaagaagtatgaagagtacatgcaaggacagaaggagaagaagaatcttcctggtgttgggtttaagaaactcaagaagattctcaagagatgcaggagaaatcatgttccttctagaatttcttttactgatgcaatcaaccacaattgttctcgtgaatgcccagtttgtgatgggacttttttcccggagcttctcaaggaaatggaagatgttgttggatggtttaacgagcatgctcagaagcttcttgagcttcatttagcttctggttttacaaagtgtcttacttggctcagaggcaacagtcgaaaaaaggaccatcatggtttgatccaagagggtaaagatttggttaattacgctctcatcaatgccgtcgccattcgaaaaatcctcaagaaatatgacaagattcatgagtctaggcaaggacaagcgtttaagactcaggtccagaaaatgcgaatagaaatccttcagtcaccgtggctctgcgagcttatggcgtttcacatcaatctgaaagaatctaagaaggaatctggagctactataacttctcctcctcctcctgttcatgcattgtttgatggttgcgctttgactttcgacgatgggaagcctttactttcctgcgagctctctgattccgtcaaagttgacattgacttgacttgttcaatatgcctggacacggtgtttgatccaatatctctaacctgcggtcacatatattgctacatgtgtgcttgctctgctgcatcagtaaacgtagttgatggcttgaaaaccgcagaagcaactgaaaaatgcccgctttgccgtgaggatggggtttataaaggtgctgttcacttggatgagctcaatattttacttaagcgaagctgcagagactattgggaagaaaggcgtaaaacagagagagcagaaaggttacaacaagcaaaggaatattgggattaccaatgccgaagcttcactggaatatga

SEQ ID NO: 2 - protein, 335 aa, Arabidopsis
MKFCKKYEEYMQGQKEKKNLPGVGFKKLKKILKRCRRNHVPSRISFTDAINHNCSRECPVCDGTFFPELLKEMEDVVGWFNEHAQKLLELHLASGFTKCLTWLRGNSRKKDHHGLIQEGKDLVNYALINAVAIRKILKKYDKIHESRQGQAFKTQVQKMRIEILQSPWLCELMAFHINLKESKKESGATITSPPPPVHALFDGCALTFDDGKPLLSCELSDSVKVDIDLTCSICLDTVFDPISLTCGHIYCYMCACSAASVNVVDGLKTAEATEKCPLCREDGVYKGAVHLDELNILLKRSCRDYWEERRKTERAERLQQAKEYWDYQCRSFTGI

SEQ ID NO: 3 - DNA, 870 bp, Arabidopsis
atgaagttttgtaagaagtatgaagagtacatgcaaggacagaaggagaagaagaatcttcctggtgttgggtttaagaaactcaagaagattctcaagagatgcaggagaaatcatgttccttctagaatttcttttactgatgcaatcaaccacaattgttctcgtgaatgcccagtttgtgatgggacttttttcccggagcttctcaaggaaatggaagatgttgttggatggtttaacgagcatgctcagaagcttcttgagcttcatttagcttctggttttacaaagtgtcttacttggctcagaggcaacagtcgaaaaaaggaccatcatggtttgatccaagagggtaaagatttggttaattacgctctcatcaatgccgtcgccattcgaaaaatcctcaagaaatatgacaagattcatgagtctaggcaaggacaagcgtttaagactcaggtccagaaaatgcgaatagaaatccttcagtcaccgtggctctgcgagcttatggcgtttcacatcaatctgaaagaatctaagaaggaatctggagctactataacttctcctcctcctcctgttcatgcattgtttgatggttgcgctttgactttcgacgatgggaagcctttactttcctgcgagctctctgattccgtcaaagttgacattgacttgacttgttcaatatgcctggatggggtttataaaggtgctgttcacttggatgagctcaatattttacttaagcgaagctgcagagactattgggaagaaaggcgtaaaacagagagagcagaaaggttacaacaagcaaaggaatattgggattaccaatgccgaagcttcactggaatatga

SEQ ID NO: 4 - protein, 289 aa, Arabidopsis
MKFCKKYEEYMQGQKEKKNLPGVGFKKLKKILKRCRRNHVPSRISFTDAINHNCSRECPVCDGTFFPELLKEMEDVVGWFNEHAQKLLELHLASGFTKCLTWLRGNSRKKDHHGLIQEGKDLVNYALINAVAIRKILKKYDKIHESRQGQAFKTQVQKMRIEILQSPWLCELMAFHINLKESKKESGATITSPPPPVHALFDGCALTFDDGKPLLSCELSDSVKVDIDLTCSICLDGVYKGAVHLDELNILLKRSCRDYWEERRKTERAERLQQAKEYWDYQCRSFTGI

SEQ ID NO: 5 - DNA, 1508 bp, Arabidopsis
agctgatatggtttgtataacaaaaccctaaaaataccgtgattaagataaaaagcaaaacaaaaccctaaaaatagaaattacaatacccaataaagttaatagcgactttaatcggtagagatttcgtcttcctccaccagcttctgttgttcttaatcactagatcccttgtttttgacgtgacgaaaagcctgagattgaaatttgtgtactatgttagaagatgaagtttggtgagacgttcacggaatatctacatggagaagaggaatggttcctggagaagtgtcgcttcgttgaatataagaaacttaagaaagtgttgaagaagtgcaagacatgtaactctacaaaatctgatgatggacaaatcatcccatctgctacttcttcatctttgtctgattcatgcgaatgcaaagcttgcccttggtgtgatcagatgttcttcgaggagctgatgaaagaagctactgacatagcgggatttttcaggtcaagagtgaggcatcttctccatcttcatgtagccactgggatgcagagatacatgattcgcctacgcagatgcttcactgacgaaaaacaagctttagtccaagagggccaaatcttgattcaatacatcaccatgaatgcaattgctatccgtaaaatcctcaagaaatatgacaaggtgcatagctcagagaatgggaagaatttcaagttgaaaatgcgagcggagcgcatagagctcttgcactcaccatggctcatagaacttggagccttttatctcaactctgggctagataatgttggaaacttcaagaattcgtttggccgtgtcgcttgtgaaaatctcaacgaagatcaaccagtgctaaaactcatgctgcctaactcaatagagctggagtatgatctaacttgtgcaatctgcctcgaaacggtattcaatccctatgctttaaagtgcggtcacatattttgtaactcgtgtgcatgctcggctgcttctgtattgattttccaaggcattaaagcagcgccccgacattcaaaatgtcccatctgcagagaggctggagtttacgcggaagcagttcacatgatcgagctgcatcttcttttaaagacacgaagcaaagaatattggaaggagaggatgatgaatgaacggtctgagatggtgaagcagtcgaagatgttttggaatgaacagacgaagcatatgatcggttattaatggtgtatctcttcaagttcaattattaacttcatctaaacaaaaggtgatgacaaaaaaaaagatatattacttttatttgtttctgccgtgcccgagacattacagattcgagagagaatcataattaggagaggtttattttattttttttctttggtgagacataaatcgagttgtgtttaaatacttttttgtgttgtttaagatgttattggatgttgttccttattaattgtttaaaagacaagtcaatagattacacacgttcatc

SEQ ID NO: 6 - protein, 335 aa, Arabidopsis
MKFGETFTEYLHGEEEWFLEKCRFVEYKKLKKVLKKCKTCNSTKSDDGQIIPSATSSSLSDSCECKACPWCDQMFFEELMKEATDIAGFFRSRVRHLLHLHVATGMQRYMIRLRRCFTDEKQALVQEGQILIQYITMNAIAIRKILKKYDKVHSSENGKNFKLKMRAERIELLHSPWLIELGAFYLNSGLDNVGNFKNSFGRVACENLNEDQPVLKLMLPNSIELEYDLTCAICLETVFNPYALKCGHIFCNSCACSAASVLIFQGIKAAPRHSKCPICREAGVYAEAVHMIELHLLLKTRSKEYWKERMMNERSEMVKQSKMFWNEQTKHMIGY

SEQ ID NO: 7 - DNA, 1696 bp, Oryza sativa
ttcgtttcaccctgctcggaaaaaaaatcctaaaagcttaatttctttctttgttcttattactgtctcacgtgagacgcctgagctcaactgaatttctggccccctcttcatctccgtgagcttcgctgccctttttctttgccttctcttggtggtttaatttcatcagttttcctccatttccttctttagccattgttccgattgccgtctcttgttgcatgtgcattccttctctgaaccgtgcccccccaattcgtccgcgagctatacaaatttggattcgttttgttcgtttccagctcaccctgtaaatcgtttctcaagtcattcaggcatactcacatcgcacaaacatatatataccccaagattttgaatccatcaatttgcagacgccataggcgtacaagttatccattcaagcgctccaaaaatttgccaagaagtttgccttaaatttggattcatgaagtttgccaagaagtacgagaagtacatgaaggggatggatgaggagctcccgggcgtggggctgaagcggctcaagaaattgctcaagaaatgccgctctgacctccaatcccacgagaacgacggctcctccgccggccgctgccccggccattgctctgtatgtgatgggagttttttcccatctcttctcaatgagatgtcagccgttattggctgcttcaatgaaaaggccaagaagcttctcgagttgcacctggcatcaggtttcaagaaatatacaatgtggttcaccagcaagggtcacaagagccatggggcattgatacagcaaggcaaagacttggttacttatgcaattataaatgccgtggccatgaggaaaattttaaagaaatatgacaagatacattactcgaagcaagggcaggaattcaaagctcaagcccagagtctgcatattgaaatacttcaatccccatggctctgtgagctgatggctttctacatgaacttgagaagaagcaagaagaacaacggtgctatggagctctttggggattgttctctcgtattcgatgatgacaaaccaacaatttcatgcaacctctttgactctatgcgtgtcgacattagcttgacatgttcgatttgcttggataccgtgtttgatccagttgctctttcctgtggtcacatatattgctacttgtgcagctgttctgctgcatccgttacgatcgttgatgggctgaagtctgccgagcgcaaatcaaagtgcccactgtgccgacaggcaggagtctttcctaatgctgtacacttggatgagctgaacatgctactaagctatagttgtccagagtactgggagaagagaatacagatggagcgggttgaacgtgttcgtctggctaaggaacactgggagtcacagtgcagggcattcttgggcatgtgattctaaagttggtggattttttttttctttagaggggcttcatatgtgatgtatagtttcttgacaaaatggaatttgccatttgttgtcacttatatcctgctctgttgctcctctttgtgtaagaagttggatttgtcatcctaaatgcaaaaacagcaggggttgttcactactgtgcacttagatgtaaatagggtagatttgtagtcattgttcaagtgcataactgtaaattcatttatatttgatcagttc

SEQ ID NO: 8 - protein, 215 aa, Oryza sativa
MKFAKKYEKYMKGMDEELPGVGLKRLKKLLKKCRSDLQSHENDGSSAGRCPGHCSVCDGSFFPSLLNEMSAVIGCFNEKAKKLLELHLASGFKKYTMWFTSKGHKSHGALIQQGKDLVTYAIINAVAMRKILKKYDKIHYSKQGQEFKAQAQSLHIEILQSPWLCELMAFYMNLRRSKKNNGAMELFGDCSLVFDDDKPTISCNLFDSMRVDISL

SEQ ID NO: 9 - DNA, 1533 bp, Oryza sativa
tccgttgcgctcgtctcctcctctcgccgcgccgccgccccccgcccccacgatcgacgactcgccgcgccgccgccgccgccgccgccaacgcatgcaggcaagatgaagttcggtgcaatatatgaagagtatcttcgggaacagcaagacaaatacctaacaaagtgctcacatgtggagtacaaacgtctcaaaaaggtactgaagaaatgtcgagttggtcgctcattgcaagaagactgccccaatggtgaccagcaggaggggaacaacgaatctccagatatttgcaaatgcaattcatgcacattgtgtgatcaaatgttctttacagaacttactaaggaggcttcagaaatagctggctgtttcagctctagagtacaacgtctcctaaatcttcatgtcccttcaggatttctacgctatatttggcgtgtaaggcaatgtttcatagatgatcaacaaatcatggttcaagaaggcagaatgttacttaattatgtaaccatgaatgctatcgctatccgtaaaattctgaagaagtatgacaaaatacatggttctgtcagtggtagagatttcaagagcaagatgcaaactgatcatattgaactgttgcagtccccttggctgatagaactgggtgctttccatctaaactgcaatagttcagatattgatgaaactgtggggttccttaagaatgagttcttcaagaatttttcctgtgatttgaccgaagcacgaccactaatgactatggctatttctgaaactatgaagtatgagtacagcctaacttgtccaatttgcttggatactttgttcaacccatatgcacttagctgtggccatctcttttgcaaaggctgtgcttgtggagctgcttctgtgtacatctttcaaggtgttaagtctgcacctcctgaggcgaagtgtcctgtatgccgatcggatggtgtctttgctcatgctgtgcatatgactgaacttgacttgctcatcaaaacaaggagcaaggattactggagacagagactgcgagaagagcggaatgagatggttaagcaatccaaagaatactgggactctcaggctatgctgtcaatgggaatttgactgagatctgaagttacaatgatcgccattcattgcttgcattggtgtcaaaccaaaaataatcaggtcgaccataatctcaagcgtggagatcatccaagatgattttaacttattaactgccagtctcctaaaagttgcaatcagtgaggactaccttgggatagcacaggctaacacagctgaggttattttattttagctcttgaagaagcaattccattccattattttttgtcctaatgttgtcgcctgatatagtttaatttggaaatgcaaccatatttaattaattttctttggaccttcaaatgtagtttggaacaatttatacgcaatttatctgttctttgctttgtaaaaacgagtgtagaaatgtcttatttgaatgtctctggctcgctgctg

SEQ ID NO: 10 - protein, 339 aa, Oryza sativa
MKFGAIYEEYLREQQDKYLTKCSHVEYKRLKKVLKKCRVGRSLQEDCPNGDQQEGNNESPDICKCNSCTLCDQMFFTELTKEASEIAGCFSSRVQRLLNLHVPSGFLRYIWRVRQCFIDDQQIMVQEGRMLLNYVTMNAIAIRKILKKYDKIHGSVSGRDFKSKMQTDHIELLQSPWLIELGAFHLNCNSSDIDETVGFLKNEFFKNFSCDLTEARPLMTMAISETMKYEYSLTCPICLDTLFNPYALSCGHLFCKGCACGAASVYIFQGVKSAPPEAKCPVCRSDGVFAHAVHMTELDLLIKTRSKDYWRQRLREERNEMVKQSKEYWDSQAMLSMGI

SEQ ID NO: 11 - DNA, 29 bp, Artificial Sequence
  Primer
acaaccggtttgagggctgaatttgtttg

SEQ ID NO: 12 - DNA, 31 bp, Artificial Sequence
  Primer
acagaattctatatcatattccagtgaagct